(19) United States
(12) Patent Application Publication
(10) Pub. No.: US 2013/0295685 A1
(43) Pub. Date: Nov. 7, 2013

(54) MOBILITY SHIFT ASSAYS FOR DETECTING ANTI-TNF ALPHA DRUGS AND AUTOANTIBODIES

(71) Applicant: NESTEC S.A., (US)

(72) Inventors: Sharat Singh, Rancho Sata Fe, CA (US); Scott Hauenstein, San diego, CA (US); Linda Ohrmund, San Diego, CA (US)

(21) Appl. No.: 13/859,664

(22) Filed: Apr. 9, 2013

Related U.S. Application Data
(63) Continuation-in-part of application No. 13/797,815, filed on Mar. 12, 2013.
(60) Provisional application No. 61/683,681, filed on Aug. 15, 2012.
(60) Provisional application No. 61/622,484, filed on Apr. 10, 2012.
(60) Provisional application No. 61/646,115, filed on May 11, 2012.
(60) Provisional application No. 61/700,855, filed on Sep. 13, 2012.
(60) Provisional application No. 61/716,415, filed on Oct. 19, 2012.
(60) Provisional application No. 61/717,592, filed on Oct. 23, 2012.

(51) Int. Cl. G01N 33/537

(52) U.S. Cl. CPC G01N 33/537
USPC 436/501

(57) ABSTRACT

The present invention provides assays for detecting and measuring the presence or level of anti-TNFα drugs and/or the autoantibodies to anti-TNFα drugs in a sample. The present invention is useful for optimizing therapy and monitoring patients receiving anti-TNFα drug therapeutics to detect the presence or level of autoantibodies against the drug. The present invention also provides methods for selecting therapy, optimizing therapy, and/or reducing toxicity in subjects receiving anti-TNFα drugs for the treatment of TNFα-mediated disease or disorders.

### CROSS-REFERENCES TO RELATED APPLICATIONS

This application is a continuation-in-part of U.S. application Ser. No. 13/797,815, filed Mar. 15, 2013 which claims priority to U.S. Provisional Application No. 61/683,681, filed Aug. 15, 2012. This application also claims priority to U.S. Application Nos. 61/622,484, filed Apr. 10, 2012; 61/646,115, filed May 11, 2012; 61/700,855, filed Sep. 13, 2012; 61/716,415, filed Oct. 19, 2012; and 61/717,592, filed Oct. 23, 2012, the disclosures all of which are hereby incorporated by reference in their entireties for all purposes.

### BACKGROUND OF THE INVENTION

Autoimmune diseases, such as Crohn's Disease (CD), ulcerative colitis (UC) and rheumatoid arthritis (RA), are characterized by a dysfunctional immune system in which the overproduction of tumor necrosis factor (TNF-α) is prevalent in the inflamed tissues. The presence of unusually high levels of proinflammatory TNF-α at the sites of inflammation is thought to drive disease pathology, and the removal of excess TNF from sites of inflammation has become a therapeutic goal.

Recombinant monoclonal antibody technology was used to develop the first generation of anti-TNF biologic agents, and in 1998 the US Food and Drug Administration (FDA) approved the use of infliximab (Remicade™) for the treatment of CD (Lee, T. W., & Fedorak, R. N. (2010). Tumor Necrosis Factor-α Monoclonal Antibodies in the Treatment of inflammatory Bowel Disease: Clinical Practice Pharmacology. Gastroenterology Clinics of North America, 39, 543-557). Infliximab is a human-murine chimeric monoclonal antibody comprised of a 25% variable murine Fab′ region linked to the 75% human IgG1:κ Fc constant region by disulfide bonds (Tracey et al., (2008). Tumor necrosis factor antagonist mechanism of action: A comprehensive review. Pharmacology and Therapeutics, 117, 244-279). Infliximab binds specifically to soluble and membrane-bound TNF-α, preventing it from binding to one of two possible receptors, TNFR1 and TNFR2 (Nesbitt et al. (2009). Certolizumab pegol: a PEGylated anti-tumour necrosis factor alpha biological agent. In F. M. Veronese (Eds.), PEGylated Protein Drugs: Basic Science and Clinical Applications (pp. 229-254). Switzerland: Birkhauser Verlag). As a bivalent mAb, infliximab can bind 2 soluble TNF trimers simultaneously, which allows multimeric complexes to form. Infliximab is known to reduce the levels of TNF-α as well as serum interleukin (IL-6) and acute-phase reactants, such as C-reactive protein (Lee, supra).

In a typical protocol for treating CD patients, infliximab is administered initially as a 5 mg/kg dose at weeks 0, 2, and 6 followed by maintenance doses of 5 mg/kg every 8 weeks. There is a wide fluctuation in serum concentrations of infliximab due to the large intravenous boluses, leading to concentration as high as 100 μg/mL upon injection. The high initial concentration is 13-40 fold greater than the peak concentrations of other TNF antagonists (Tracey et al., supra). Infliximab has a low clearance rate (t_{1/2}=8-10 days) that appears to be independent of typical drug-metabolizing enzymes and is most likely caused by nonspecific proteases. The clinical response is strongly correlated with serum concentrations, and it is likely that antibody formation to infliximab decreases serum levels to non-detectable levels. The variable murine region is thought to be the antigenic component that causes the formation of “antibodies to infliximab” or ATI. Not only does development of ATI lead to increased drug clearance, but it could also result in a range of adverse reactions from mild allergic response to anaphylactic shock. Many patients do not respond to infliximab therapy, and require higher doses or dosing frequency adjustments due to lack of sufficient response (Tracey et al., supra). Furthermore, many patients with secondary response failure to one anti-TNF-α drug benefit from switching to other anti-TNF-α drugs, suggesting a role of neutralizing antibodies.

ELISA assays are currently used to monitor both infliximab and ATI levels in patient serum samples. Typically, the infliximab ELISA utilizes a 96-well microplate ELISA with recombinant TNFα passively adsorbed onto the plate to form the solid phase. The ATI Bridge ELISA employs Infliximab as both capture and detector. While the ELISA assays are robust and sensitive, they have several shortcomings that need to be addressed. Solid phase assays are prone to artifacts such as constraints on the bound antigen that limit its ability to interact with its target, often leading to decreased binding affinity. In the case of the infliximab ELISA assay, this limitation prevents detection of total infliximab in circulation. Only free infliximab can be detected, preventing analysis of patient serum with moderate to high ATI levels. Similarly, only free ATI can be detected in the Bridge ELISA, preventing the detection of total ATI in circulation.

In view of the foregoing, there is a need for new assays to measure anti-TNFα drugs as well as the presence or level of an autoantibody to an anti-TNFα drug. The present invention satisfies these and other needs.

### BRIEF SUMMARY OF THE INVENTION

The present invention provides assays for detecting and measuring the presence or concentration level of an anti-TNFα drug in a sample. The present invention is useful for optimizing therapy and monitoring patients receiving anti-TNFα drugs to detect their presence and serum concentration levels. In addition, assays are provided herein to detect the presence and measure the amount of autoantibodies (e.g., HACA and/or HAHA) against the drug. The present invention also provides methods for selecting therapy, optimizing therapy, and/or reducing toxicity in subjects receiving anti-TNFα drugs for the treatment of TNFα-mediated diseases or disorders (e.g., inflammatory bowel disease, rheumatoid arthritis, and the like).

In one embodiment, the present invention provides a method for determining the presence or level of an anti-TNFα drug in a sample, comprising:
(a) contacting a labeled TNFα with a sample having an anti-TNFα drug to form a labeled complex with the anti-TNFα drug;
(b) subjecting the labeled complex to size exclusion chromatography to separate the labeled complex from free labeled TNFα and to measure the amount of the labeled complex and the amount of free labeled TNFα;
(c) calculating a ratio of the amount of the labeled complex to the sum of the labeled complex plus free labeled TNFα; and
(d) comparing the ratio calculated in step (c) to a standard curve of known amounts of the anti-TNFα drug, thereby determining the presence or level of the anti-TNFα drug.

In another embodiment, the present invention provides a method for determining the presence or level of an autoantibody to an anti-TNFα drug in a sample, comprising:
(a) contacting a labeled anti-TNFα drug with the sample to form a labeled complex with the autoantibody;
(b) subjecting the labeled complex to size exclusion chromatography to separate the labeled complex from free labeled anti-TNFα drug and to measure the amount of the labeled complex and the amount of the free labeled anti-TNFα drug;
(c) calculating a ratio of the amount of the labeled complex to the sum of the amount of the labeled complex plus free labeled anti-TNFα drug; and
(d) comparing the ratio calculated in step (c) to a standard curve of known amounts of the autoantibody, to thereby determine the presence or level of the autoantibody.

In some embodiments, the present invention provides a method to determine the total amount of autoantibody in a sample. This total amount of autoantibody in a sample is the sum of autoantibody bound to unlabeled anti-TNFα drug plus the amount of autoantibody bound to labeled anti-TNFα drug. As such, in one embodiment, the present invention provides a method for determining the total amount of an autoantibody in a sample, comprising:
(a) determining the level of autoantibody bound to labeled anti-TNFα drug according to the following method:
(i) contacting a labeled anti-TNFα drug with the sample to form a labeled complex with the autoantibody;
(ii) subjecting the labeled complex to size exclusion chromatography to separate the labeled complex from free labeled anti-TNFα drug and to measure the amount of the labeled complex and the amount of the free labeled anti-TNFα drug;
(iii) calculating a ratio of the amount of the labeled complex to the sum of the amount of the labeled complex plus free labeled anti-TNFα drug;
(iv) comparing the ratio calculated in step (iii) to a standard curve of known amounts of the autoantibody, to thereby determine the presence or level of the autoantibody, as being the amount of autoantibody bound to a labeled anti-TNFα drug; and
(b) adding the amount of autoantibody bound to unlabeled anti-TNFα drug to the level determined in step (iv) to produce the total amount of autoantibody in the sample.

Accordingly, in some aspects, the methods of the invention provide information useful for guiding treatment decisions for patients receiving or about to receive anti-TNFα drug therapy, e.g., by selecting an appropriate anti-TNFα therapy for initial treatment, by determining when or how to adjust or modify (e.g., increase or decrease) the subsequent dose of an anti-TNFα drug, by determining when or how to combine an anti-TNFα drug (e.g., at an initial, increased, decreased, or same dose) with one or more immunosuppressive agents such as methotrexate (MTX) and/or azathioprine (AZA), and/or by determining when or how to change the current course of therapy (e.g., switch to a different anti-TNFα drug or to a drug that targets a different mechanism such as an IL-6 receptor-inhibiting monoclonal antibody).

These and other objects, features, and advantages of the present invention will become more apparent when read with the following detailed description and figures which follow.

### BRIEF DESCRIPTION OF THE DRAWINGS

FIG. 1A and FIG. 1B show exemplary embodiments of the assays of the present invention wherein size exclusion HPLC is used to detect binding. FIG. 1A shows a chromatogram of TNFα-Alexa488 and a control. FIG. 1B shows a chromatogram of TNFα-Alexa488 plus infliximab.

FIG. 2 shows an example of a standard curve for an infliximab HPLC mobility shift assay.

FIGS. 3A-3D show exemplary embodiments of the assays of the present invention. FIG. 3A shows a chromatogram of 37.5 ng Infliximab-Alexa488. FIG. 3B shows 37.5 ng Infliximab-Alexa488 plus 1% ATI Positive Serum. FIG. 3C shows a chromatogram of ADL-Alexa488 and a control. FIG. 3D shows a chromatogram of adalimumab-Alexa488 plus ATA.

FIG. 4 shows an example of a standard curve for an autoantibody to infliximab HPLC mobility shift assay.

FIG. 5 shows an example of a standard curve for an autoantibody to adalimumab HPLC mobility shift assay.

FIGS. 6A-6B show a schematic illustration of the principles of the (A) ATI-HMSA and (B) IFX-HMSA. In FIG. 6A, fluorescently-labeled IFX (IFX-488; MW˜150 kD) is incubated with serum samples containing ATI (MW˜150-900 kD). The newly-formed immune complexes of ATI/IFX-488 have significantly higher MW than the free IFX-488, and can be separated and quantified by SE-HPLC with fluorescent detection. In FIG. 6B, fluorescently-labeled TNFα (TNF-488, MW˜51 kD) binds to IFX in serum samples and the newly-formed immune complexes with increased MW of 51 kD to >200 kD can then be separated and quantified.

FIG. 7A-B illustrate overlapping SE-HPLC chromatograms of (A) the ATI calibration standards and (B) the ATI-HMSA-generated standard curve. In FIG. 7A, the greater the amount of ATI calibration standard added to the IFX-488 reaction mixture, the greater the shift of free IFX488 (ca. 10-11.5 min) towards the formation of immune complexes (ca. 6.7-9.5 min); however, there were no changes observed for the internal control (14 min). In FIG. 7B, the standard curve was generated by plotting the ratios of the proportion of the shifted area over total area vs. the concentration of ATI in the reaction mixture. LU=luminescent units.

FIG. 8A-B illustrate overlapping SE-HPLC chromatograms of (A) the IFX calibration standards and (B) the IFX-HMSA-generated standard curve. In FIG. 8A, the greater the amount of IFX calibration standard added to the TNF-488 reaction mixture, the greater the shift of free TNF-488 (ca. 11.5-12.5 min) towards the formation of immune complexes (ca. 6.7-9.5 min); however, no changes were observed for the internal control (14 min). In FIG. 8B, the IFX-HMSA standard curve was generated using the same method as the ATI-HMSA. LU=luminescent units.

FIG. 9A-B show the linearity of dilution for the (A) ATI-HMSA and (B) IFX-HMSA. Linearity of the ATI-HMSA and the IFX-HMSA were determined by a 2-fold serial dilution of a high-titer ATI-positive sample and a high concentration IFX-positive sample, respectively. The relationships between the observed and the expected concentrations were plotted. FIGS. 9A and B show that the R² value and the slope of each linear regression curve demonstrate good linearity.

FIG. 10 depicts ATI-HMSA drug tolerance. Interference by IFX in the ATI-HMSA was assessed by adding increasing doses of IFX (6.6, 20, and 60 μg/mL) in each of the eight ATI calibration standards to determine their effects on the generation of the standard curve. The results showed that the ATI-HMSA detecst an ATI level as low as 0.036 μg/mL in the presence of 60 μg/mL of IFX.

FIG. 11A-D illustrate the clinical sample test and the assay cut point determination for the ATI-HMSA. Serum samples from healthy donors and patients with IBD were analyzed by the ATI-HMSA (cut point value was calculated as the mean value plus 2.0×SD). FIG. 11A shows individual data from 100 healthy samples on the proportion of shifted area/total area obtained from the analysis. FIG. 11B shows the interpolated ATI values for the healthy samples. FIG. 11C shows the individual data from 100 serum samples from patients with IBD on the proportion of shifted area/total area obtained from the analysis. FIG. 11D shows the interpolated ATI values for the IBD samples.

FIG. 12A shows ATI concentrations in healthy control and IBD patient serum samples determined by the ATI-HMSA. The horizontal dotted line represents the cut point and the horizontal solid line represents the mean, the y-axis scale is Log 2.

FIG. 12B represents a plot of the Receiver Operating Characteristic (ROC) curve using data obtained from the ATI-HMSA analysis of healthy control and IBD patient serum samples.

FIG. 13 shows the correlation of the ATI-HMSA and bridging ELISA on the measurement of ATI in IBD patient serum samples.

FIG. 14 shows a tabulation of characteristics of the ATI-HMSA standard curve.

FIG. 15 shows a tabulation of characteristics of the IFX-HMSA standard curve.

FIG. 16 shows a tabulation of an assay precision of the ATI-HMSA assay.

FIG. 17 shows a tabulation of an assay precision of the IFX-HMSA assay.

FIG. 18 shows the effects of potential interfering substances in the ATI-HMSA and IFX-HMSA. Potential interference in the presence of common endogenous components of human serum and drug taken by the patients was tested by spiking in each of the substances in the three QC samples (high, mid and low) and determining their recovery in the two assays. No significant interference was observed among any of the spiked-in substances as the recovery of the QC samples was close to 100%. In regard to the effects of TNFα, TNFβ, sTNFR1 and TNFR2, interference occurred only at very high concentrations of these substances which are unlikely to be encountered in the patient serum. All recovery values shown are from the medium controls of each assay.

FIG. 19 represents an analysis of ATI in the presence of IFX as detected by HMSA or ELISA. ATI HMSA continues to dilute linearly in the presence of 14 (circle) or 60 (triangle) μg/mL IFX. The ELISA assay (square and diamond) does not reliably detect any sample containing IFX.

FIG. 20 represents an analysis of IFX in the presence of ATI as detected by HMSA or ELISA. IFX was spiked into ATI positive serum at a concentration of 14 μg/mL. IFX HMSA returns accurate values for IFX in the presence of up ˜10 μg/mL (65 U/mL) ATI and outperforms ELISA assay across all concentrations.

FIG. 21A-B show the dynamic range of ATI- and IFX-HMSA assays. The ATI HMSA diluted linearly from 0.56-30 μg/mL (0-200 U/mL), whereas the ATI ELISA assay overestimated the amount of ATI at low concentrations and did not accurately detect higher levels of ATI due to its low dynamic range (FIG. 21A). Both the HMSA and ELISA detected IFX, however the HMSA more accurately detected IFX across the range shown (1-40 μg/mL) (FIG. 21B).

FIG. 22 shows an example of an IFX high sensitivity mobility shift assay standard curve.

FIG. 23 shows SEC-HPLC chromatograms of IFX high sensitivity mobility shift assay. The more IFX in the reaction, the more shift of free TNF-Alexa488 to form the immune-complex. No changes are noticed on the internal control.

FIG. 24A-D show measurements of infliximab, adalimumab and other anti-TNF biologics (GLM, golimumab; ETN, etanercept). FIG. 24A shows standard curves for each anti-TNF. Each drug has a LLOQ of approximately 1.0 μg/mL. FIG. 24B shows adalimumab levels in 81 IBD patients receiving adalimumab therapy and 23 healthy donors. FIG. 24C shows a histogram of the IBD patients tested. The number above each bar represents the number of patients positive for antibody-to-adalimumab (ATA). FIG. 24D shows a summary table of the IBD patients tested.

FIG. 25 shows a graph of the percentage of subjects who are ATI positive and either below or at least at the IFX cut-off. ATI positive subjects are more likely to have IFX levels less than 3 μg/ml. When IFX levels were 3 μg/ml or higher, the percentage of ATI positive was lower.

FIG. 26 illustrates the relationship between the presence of ATI and IFX in the cohort. The data shows that ATI negative subjects are more likely to have IFX levels greater or equal to 3 μg/ml. ATI positive subjects are more likely to have IFX leves less than 3 μg/ml.

FIG. 27A-B illustrate standard curves for the ATA-HMSA (A) and the adalimumab-HMSA (B). Serial dilutions of the ATA calibration standards (FIG. 27A) or adalimumab calibration standards (FIG. 27B) were incubated with adalimumab-AlexaFluor-488 or TNF-α-AlexaFluor-488, respectively, which dose-dependently formed immune complexes. Immune complexes and remaining free adalimumab-AlexaFluor-488 or TNF-α-AlexaFluor-488 were resolved by SE-HPLC analysis. An exponential association standard curve was generated from the calibration standards.

FIG. 28 illustrates ATA-HMSA drug tolerance. Interference by adalimumab in the ATA-HMSA was assessed by increasing doses of adalimumab (1.25-40 μg/mL) in each of the three different ATA concentrations (10, 30, and 80 U/mL). ATA-HMSA is able to detect an ATA level as low as 10 U/mL in the presence of up to 20 μg/mL of adalimumab.

FIG. 29 illustrates a histogram showing the distribution of adalimumab levels in patients who have lost response to treatment. Higher frequency was observed in the lower adalimumab concentration range from patients who had lost response to drug treatment.

FIG. 30A-B illustrate the relationship between adalimumab and ATA levels in serum samples from patients treated with adalimumab. Adalimumab and ATA concentrations were obtained from HMSA. FIG. 30A shows a plot that illustrates the relationship between ATA positivity and adalimumab concentration. Concentrations of ATA and adalimumab from each individual IBD patient (triangle), RA patient (circle), and PS patient (diamond) were plotted (FIG. 30B). The vertical dashed line is the cut point for adalimumab (0.68 μg/mL), and the horizontal solid line is the cut point for ATA (0.55 U/mL).

FIG. 31 illustrates ATA levels in patients with different diseases. ATA concentrations in serum from patients with different diseases were plotted. There was no significant difference in average ATA concentrations or ATA positivity (above the cut point of 0.55 U/mL, horizontal solid line) among IBD, RA, and PS patients who had lost response to adalimumab therapy.

### DETAILED DESCRIPTION OF THE INVENTION

### I. Definitions

As used herein, the following terms have the meanings ascribed to them unless specified otherwise.

The terms “anti-TNFα drug” or “TNFα inhibitor” as used herein is intended to encompass agents including proteins, antibodies, antibody fragments, fusion proteins (e.g., Ig fusion proteins or Fc fusion proteins), multivalent binding proteins (e.g., DVD Ig), small molecule TNFα antagonists and similar naturally- or normaturally-occurring molecules, and/or recombinant and/or engineered forms thereof, that, directly or indirectly, inhibit TNFα activity, such as by inhibiting interaction of TNFα with a cell surface receptor for TNFα, inhibiting TNFα protein production, inhibiting TNFα gene expression, inhibiting TNFα secretion from cells, inhibiting TNFα receptor signaling or any other means resulting in decreased TNFα activity in a subject. The term “anti-TNFα drug” or “TNFα inhibitor” preferably includes agents which interfere with TNFα activity. Examples of anti-TNFα drugs include, without limitation, infliximab (REMICADE™, Johnson and Johnson), human anti-TNF monoclonal antibody adalimumab (D2E7/HUMIRA™, Abbott Laboratories), etanercept (ENBREL™, Amgen), certolizumab pegol (CIMZIA®, UCB, Inc.), golimumab (SIMPONI®; CNTO 148), CDP 571 (Celltech), CDP 870 (Celltech), as well as other compounds which inhibit TNFα activity, such that when administered to a subject suffering from or at risk of suffering from a disorder in which TNFα activity is detrimental (e.g., RA), the disorder is treated.

The term “TNFα” is intended to include a human cytokine that exists as a 17 kDa secreted form and a 26 kDa membrane associated form, the biologically active form of which is composed of a trimer of noncovalently bound 17 kDa molecules. The structure of TNFα is described further in, for example, Jones et al., Nature, 338:225-228 (1989). The term TNFα is intended to include human TNFα, a recombinant human TNFα (rhTNF-α), or TNFα that is at least about 80% identity to the human TNFα protein. Human TNFα consists of a 35 amino acid (aa) cytoplasmic domain, a 21 aa transmembrane segment, and a 177 aa extracellular domain (ECD) (Pennica, D. et al. (1984) Nature 312:724). Within the ECD, human TNFα shares 97% aa sequence identity with rhesus TNFα, and 71% to 92% aa sequence identity with bovine, canine, cotton rat, equine, feline, mouse, porcine, and rat TNFα. TNFα can be prepared by standard recombinant expression methods or purchased commercially (R & D Systems, Catalog No. 210-TA, Minneapolis, Minn.).

In certain embodiments, “TNFα” is an “antigen,” which includes a molecule or a portion of the molecule capable of being bound by an anti-TNF-α drug. TNFα can have one or more than one epitope. In certain instances, TNFα will react, in a highly selective manner, with an anti-TNFα antibody. Preferred antigens that bind antibodies, fragments, and regions of anti-TNFα antibodies include at least 5 amino acids of human TNFα. In certain instances, TNFα is a sufficient length having an epitope of TNFα that is capable of binding anti-TNFα antibodies, fragments, and regions thereof.

The term “predicting responsiveness to an anti-TNFα drug” is intended to refer to an ability to assess the likelihood that treatment of a subject with an anti-TNFα drug will or will not be effective in (e.g., provide a measurable benefit to) the subject. In particular, such an ability to assess the likelihood that treatment will or will not be effective typically is exercised after treatment has begun, and an indicator of effectiveness (e.g., an indicator of measurable benefit) has been observed in the subject. Particularly preferred anti-TNFα drugs are biologic agents that have been approved by the FDA for use in humans in the treatment of TNFα-mediated diseases or disorders and include those anti-TNFα drugs described herein.

The term “size exclusion chromatography” or “SEC” includes a chromatographic method in which molecules in solution are separated based on their size and/or hydrodynamic volume. It is applied to large molecules or macromolecular complexes such as proteins and their conjugates. Typically, when an aqueous solution is used to transport the sample through the column, the technique is known as gel filtration chromatography.

The terms “complex,” “immuno-complex,” “conjugate,” and “immunoconjugate” include, but are not limited to, TNFα bound (e.g., by non-covalent means) to an anti-TNFα drug, an anti-TNFα drug bound (e.g., by non-covalent means) to an autoantibody against the anti-TNFα drug, and an anti-TNFα drug bound (e.g., by non-covalent means) to both TNFα and an autoantibody against the anti-TNFα drug.

As used herein, an entity that is modified by the term “labeled” includes any entity, molecule, protein, enzyme, antibody, antibody fragment, cytokine, or related species that is conjugated with another molecule or chemical entity that is empirically detectable. Chemical species suitable as labels for labeled-entities include, but are not limited to, fluorescent dyes, e.g. Alexa Fluor® dyes such as Alexa Fluor® 647, Alexa Fluor® 488, quantum dots, optical dyes, luminescent dyes, and radionuclides, e.g. ¹²⁵I. Additional labels are described in further detail below.

The term “effective amount” includes a dose of a drug that is capable of achieving a therapeutic effect in a subject in need thereof as well as the bioavailable amount of a drug. The term “bioavailable” includes the fraction of an administered dose of a drug that is available for therapeutic activity. For example, an effective amount of a drug useful for treating diseases and disorders in which TNF-α has been implicated in the pathophysiology can be the amount that is capable of preventing or relieving one or more symptoms associated therewith.

The phrase “fluorescence label detection” includes a means for detecting a fluorescent label. Means for detection include, but are not limited to, a spectrometer, a fluorimeter, a photometer, and a detection device commonly incorporated with a chromatography instrument such as, but not limited to, size exclusion-high performance liquid chromatography, such as, but not limited to, an Agilent-1200 HPLC System.

The phrase “optimize therapy” includes optimizing the dose (e.g., the effective amount or level) and/or the type of a particular therapy. For example, optimizing the dose of an anti-TNFα drug includes increasing or decreasing the amount of the anti-TNFα drug subsequently administered to a subject. In certain instances, optimizing the type of an anti-TNFα drug includes changing the administered anti-TNFα drug from one drug to a different drug (e.g., a different anti-TNFα drug). In other instances, optimizing therapy includes co-administering a dose of an anti-TNFα drug (e.g., at an increased, decreased, or same dose as the previous dose) in combination with an immunosuppressive drug.

The term “co-administer” includes to administer more than one active agent, such that the duration of physiological effect of one active agent overlaps with the physiological effect of a second active agent.

The term “subject,” “patient,” or “individual” typically refers to humans, but also to other animals including, e.g., other primates, rodents, canines, felines, equines, ovines, porcines, and the like.

The term “course of therapy” includes any therapeutic approach taken to relieve or prevent one or more symptoms associated with a TNFα-mediated disease or disorder. The term encompasses administering any compound, drug, procedure, and/or regimen useful for improving the health of an individual with a TNFα-mediated disease or disorder and includes any of the therapeutic agents described herein. One skilled in the art will appreciate that either the course of therapy or the dose of the current course of therapy can be changed (e.g., increased or decreased) based upon the presence or concentration level of TNFα, anti-TNFα drug, and/or anti-drug antibody using the methods of the present invention.

The term “immunosuppressive drug” or “immunosuppressive agent” includes any substance capable of producing an immunosuppressive effect, e.g., the prevention or diminution of the immune response, as by irradiation or by administration of drugs such as anti-metabolites, anti-lymphocyte sera, antibodies, etc. Examples of immunosuppressive drugs include, without limitation, thiopurine drugs such as azathioprine (AZA) and metabolites thereof; anti-metabolites such as methotrexate (MTX); sirolimus (rapamycin); temsirolimus; everolimus; tacrolimus (FK-506); FK-778; anti-lymphocyte globulin antibodies, anti-thymocyte globulin antibodies, anti-CD 3 antibodies, anti-CD4 antibodies, and antibody-toxin conjugates; cyclosporine; mycophenolate; mizoribine monophosphate; scoparone; glatiramer acetate; metabolites thereof; pharmaceutically acceptable salts thereof; derivatives thereof; prodrugs thereof; and combinations thereof.

The term “thiopurine drug” includes azathioprine (AZA), 6-mercaptopurine (6-MP), or any metabolite thereof that has therapeutic efficacy and includes, without limitation, 6-thioguanine (6-TG), 6-methylmercaptopurine riboside, 6-thioinosine nucleotides (e.g., 6-thioinosine monophosphate, 6-thioinosine diphosphate, 6-thioinosine triphosphate), 6-thioguanine nucleotides (e.g., 6-thioguanosine monophosphate, 6-thioguanosine diphosphate, 6-thioguanosine triphosphate), 6-thioxanthosine nucleotides (e.g., 6-thioxanthosine monophosphate, 6-thioxanthosine diphosphate, 6-thioxanthosine triphosphate), derivatives thereof, analogues thereof, and combinations thereof.

The term “sample” includes any biological specimen obtained from an individual. Samples include, without limitation, whole blood, plasma, serum, red blood cells, white blood cells (e.g., peripheral blood mononuclear cells (PBMC), polymorphonuclear (PMN) cells), ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by random periareolar fine needle aspiration), any other bodily fluid, a tissue sample such as a biopsy of a site of inflammation (e.g., needle biopsy), cellular extracts thereof, and an immunoglobulin enriched fraction derived from one or more of these bodily fluids or tissues. In some embodiments, the sample is whole blood, a fractional component thereof such as plasma, serum, or a cell pellet, or an immunoglobulin enriched fraction thereof. One skilled in the art will appreciate that samples such as serum samples can be diluted prior to the analysis. In certain embodiments, the sample is obtained by isolating PBMCs and/or PMN cells using any technique known in the art. In certain other embodiments, the sample is a tissue biopsy such as, e.g., from a site of inflammation such as a portion of the gastrointestinal tract or synovial tissue.

### II. Embodiments

The present invention provides assays for detecting and measuring the presence or level of an anti-TNFα drug and/or the presence or level of autoantibodies to anti-TNFα drugs in a sample. In one aspect, the present invention provides assays for detecting and measuring the presence or level of infliximab (IFX) and/or the presence or level of autoantibodies to infliximab (ATI) in a sample. In another aspect, the present invention provides assays for detecting and measuring the presence or level of adalimumab (ADL) and/or the presence or level of autoantibodies to adalimumab (ATA) in a sample. The present invention is useful for optimizing therapy and monitoring patients receiving anti-TNFα drug therapeutics to detect the presence or level of autoantibodies (e.g., HACA and/or HAHA) against the drug. The present invention also provides methods for selecting therapy, optimizing therapy, and/or reducing toxicity in subjects receiving anti-TNFα drugs for the treatment of TNFα-mediated disease or disorders.

The following applications disclose related technology and are hereby incorporated by reference in their entirety for all purposes: US Patent App. Pub. No. US 2012/329172 and International App. Pub. Nos. WO 2012/054532 and WO 2013/006810.

A. Assay for an Anti-TNFα Drug

In one embodiment, the present invention provides a method for determining the presence or level of an anti-TNFα drug in a sample, comprising:
(a) contacting a labeled TNFα with a sample having an anti-TNFα drug to form a labeled complex with the anti-TNFα drug;
(b) subjecting the labeled complex to size exclusion chromatography to separate the labeled complex from free labeled TNFα and to measure the amount of the labeled complex and the amount of the free labeled TNFα;
(c) calculating a ratio of the amount of the labeled complex to the sum of the labeled complex plus free labeled TNFα; and
(d) comparing the ratio calculated in step (c) to a standard curve of known amounts of the anti-TNFα drug, thereby determining the presence or level of the anti-TNFα drug.

In certain aspects, the assay is performed by incubating fluorescently labeled recombinant TNF-α (e.g., TNF-α Alexa488) and optionally containing a deactivated Alexa488 loading control with a sample such as serum containing infliximab, which is allowed to reach equilibrium, to form various complexes of increasing molecular weight. Complexes are formed ranging in size from approximately 200 kDa for 1:1 binding to over 2000 kDa.

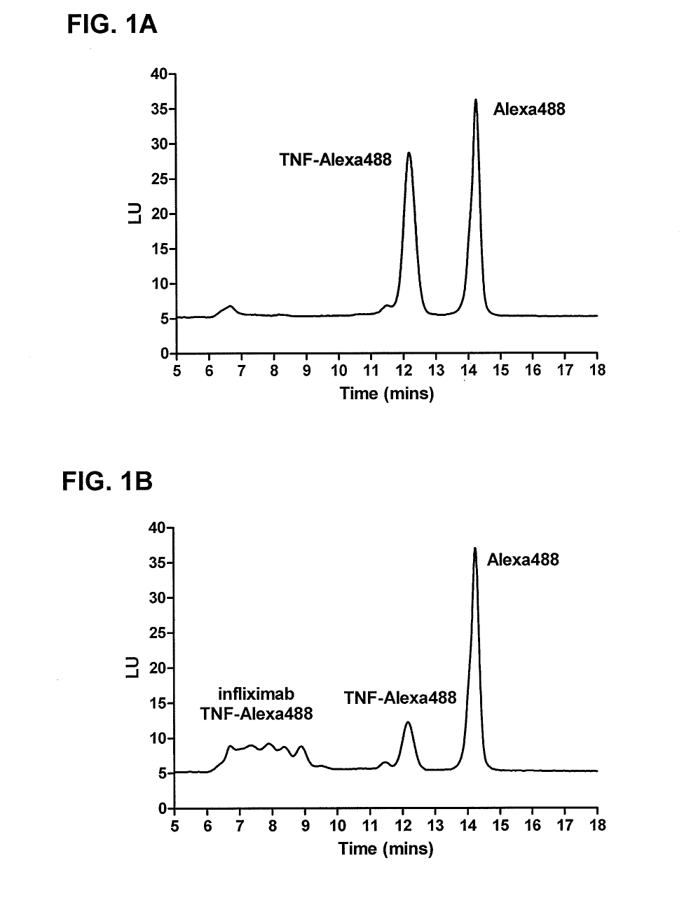

As shown in FIG. 1, after injection and elution of the complex mixture through a column packed with, for example, a gel media, free TNFα-Alexa488 (Mw˜51 kDa) elutes at a retention time (Rₜ) of approximately 11-12.5 minutes (FIG. 1A) while infliximab-TNFα-Alexa488 complexes (FIG. 1B) elute at the range from 6-10 minutes, and the deactivated Alexa488 loading control elutes at about 13.5-14.5 minutes. The assay of the present invention resolves infliximab-TNFα complexes from free TNFα based on the size of the complexes formed. Preferably, the labeled complex is eluted first, followed by the free labeled TNFα.

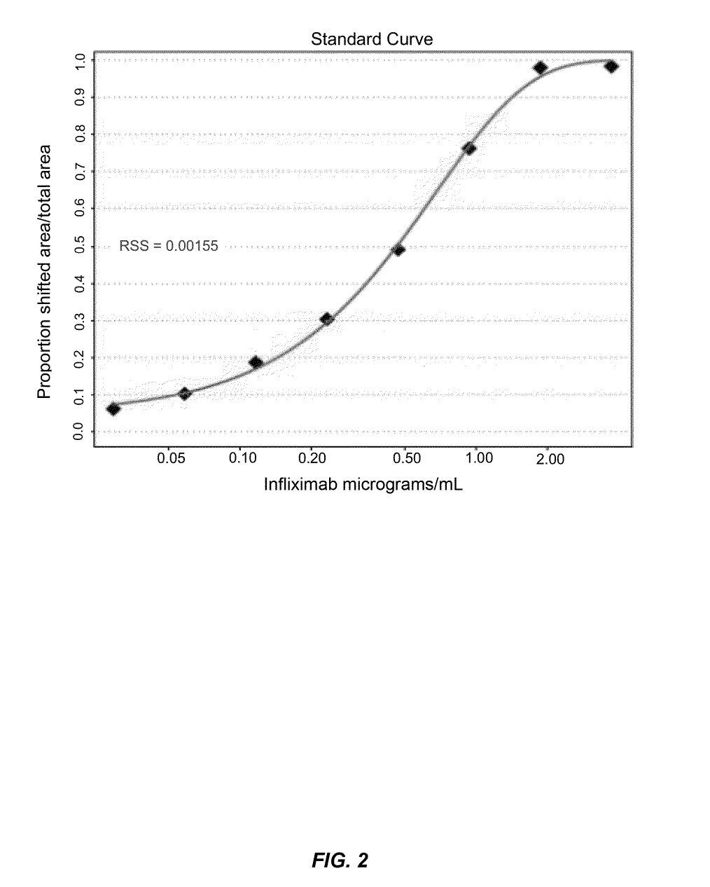

As shown in FIG. 1, quantification can be performed by tracking the appearance of high molecular weight peaks (infliximab-TNFα-Alexa488 complexes (FIG. 1B)) and/or the disappearance of the free labeled TNFα peak (Rₜ=11-12.5 min). FIG. 2 shows an exemplary standard curve generated when the y-axis comprises a ratio, wherein the ratio has a numerator which is labeled complex (e.g., labeled TNFα bound to an anti-TNFα drug) and a denominator which is the sum of the labeled complex plus free labeled TNFα. The x-axis comprises known amounts of the anti-TNFα drug (e.g., IFX, ADL, and the like)

The infliximab standard curve and positive controls are prepared by diluting infliximab in normal human serum. In certain instances, standard samples (e.g., 0.73 to 46.88 μg/mL) and high (e.g., 15.63 μg/mL), medium (e.g., 7.81 μg/mL) and low (e.g., 3.91 μg/mL) infliximab positive controls are run during each assay.

Quantification of the infliximab assay is performed by tracking the appearance of high molecular weight peaks (Rₜ=6-10 min) or the disappearance of the free labeled TNF peak (Rₜ=11-12.5 min). Preferably, raw chromatograms are collected in automated analysis. The fraction of the shifted area representing infliximab-TNF-Alexa488 complexes is plotted from an infliximab standard curve and fitted with a 5-parameter logistic model to account for asymmetry.

In certain instances, the areas under the bound TNFα peak, free TNFα peak and control peak are found by integrating the peak areas. The proportion of the TNFα peak area shifted to bound from free is then calculated for each sample by using the following formula:

p_{D}=b_{D}/(b_{D}+f_{D})

Where p_{D}=proportion of shifted area, b_{D}=area under the bound TNFα-infliximab peak, and f_{D}=the area under the free TNFα peak. Optionally, the ratio of free peak area to control peak area is also calculated. The standard curve has a y-axis of p_{D}, and an x-axis of known amounts of anti-TNFα drug. Using the standard curve, concentrations of control samples, unknown samples, and test samples are interpolated and determined.

Suitable anti-TNFα drugs include, but are not limited to, REMICADE™ (infliximab), ENBREL™ (etanercept), HUMIRA™ (adalimumab), CIMZIA® (certolizumab pegol), and combinations thereof. In one preferred embodiment, the anti-TNFα drug is REMICADE™ (infliximab). In another preferred embodiment, the anti-TNFα drug is CIMZIA® (adalimumab). As a skilled artisan will appreciate, the steps of the foregoing and following methods do not necessarily have to be performed in the particular order in which they are presented.

In particular embodiments, the presence of an anti-TNFα antibody is determined by comparing the level of labeled complex to a cut point (e.g., cut-off value). In certain instances, the cut point is about 0.10, 0.15, 0.20, 0.25, 0.30, 0.35, 0.40, 0.45, 0.50, 0.55, 0.60, 0.65, 0.70, 0.75, 0.80, 0.85, 0.90, 0.91, 0.92, 0.93, 0.94, 0.95, 0.96, 0.97, 0.98, 0.99, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.10, 2.20, 2.30, 2.40, 2.50, 2.60, 2.70, 2.80, 2.90, 3.00, 3.50, 4.00, 4.50, 5.00, 5.50, 6.00, 6.50, 7.00, 7.50, 8.00, 8.50, 9.00, 9.50, 10.00, or more μg/ml. In certain instances, a level (concentration or amount) of labeled complex that is equal to or greater than the cut point indicates that the sample is positive for the anti-TNFα antibody. In other instances, a particular cut point can be set to provide or yield a specific percentage of a clinical parameter such as sensitivity, specificity, negative predictive value, positive predictive value, overall accuracy, and combinations thereof. As a non-limiting example, the cut point for assays of the present invention for detecting the presence of an anti-TNFα antibody such as REMICADE™ (infliximab) can be about 0.98 μg/ml, and can provide a clinical specificity of about 95%.

B. Assay for Autoantibody to Anti-TNFα Drug

In another embodiment, the present invention provides a method for determining the presence or level of an autoantibody to an anti-TNFα drug in a sample, comprising:
(a) contacting a labeled anti-TNFα drug with the sample to form a labeled complex with the autoantibody;
(b) subjecting the labeled complex to size exclusion chromatography to separate the labeled complex from free labeled anti-TNFα drug and to measure the amount of the labeled complex and the amount of the free labeled anti-TNFα drug;
(c) calculating a ratio of the amount of the labeled complex to the sum of the amount of the labeled complex plus free labeled anti-TNFα drug; and
(d) comparing the ratio calculated in step (c) to a standard curve of known amounts of the autoantibody, to thereby determine the presence or level of the autoantibody.

In some embodiments, prior to step (a) the sample is contacted with an acid to dissociate any anti-TNFα drug bound to an autoantibody against the anti-TNFα drug in the sample. In certain instances, the acid comprises an organic acid. In other embodiments, the acid comprises an inorganic acid. In further embodiments, the acid comprises a mixture of an organic acid and an inorganic acid. Non-limiting examples of organic acids include citric acid, isocitric acid, glutamic acid, acetic acid, lactic acid, formic acid, oxalic acid, uric acid, trifluoroacetic acid, benzene sulfonic acid, aminomethanesulfonic acid, camphor-10-sulfonic acid, chloroacetic acid, bromoacetic acid, iodoacetic acid, propanoic acid, butanoic acid, glyceric acid, succinic acid, malic acid, aspartic acid, and combinations thereof. Non-limiting examples of inorganic acids include hydrochloric acid, nitric acid, phosphoric acid, sulfuric acid, boric acid, hydrofluoric acid, hydrobromic acid, and combinations thereof.

In certain embodiments, the amount of an acid corresponds to a concentration of from about 0.01M to about 10M, about 0.1M to about 5M, about 0.1M to about 2M, about 0.2M to about 1M, or about 0.25M to about 0.75M of an acid or a mixture of acids. In other embodiments, the amount of an acid corresponds to a concentration of greater than or equal to about 0.01M, 0.05M, 0.1M, 0.2M, 0.3M, 0.4M, 0.5M, 0.6M, 0.7M, 0.8M, 0.9M, 1M, 2M, 3M, 4M, 5M, 6M, 7M, 8M, 9M, or 10M of an acid or a mixture of acids. The pH of the acid can be, for example, about 0.1, 0.5, 1.0, 1.5, 2.0, 2.5, 3.0, 3.5, 4.0, 4.5, 5.0, 5.5, 6.0, or 6.5.

In some embodiments, the sample is contacted with an acid an amount of time that is sufficient to dissociate preformed complexes of the autoantibody and the anti-TNFα drug. In certain instances, the sample is contacted (e.g., incubated) with an acid for a period of time ranging from about 0.1 hours to about 24 hours, about 0.2 hours to about 16 hours, about 0.5 hours to about 10 hours, about 0.5 hours to about 5 hours, or about 0.5 hours to about 2 hours. In other instances, the sample is contacted (e.g., incubated) with an acid for a period of time that is greater than or equal to about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, or 10 hours. The sample can be contacted with an acid at 4° C., room temperature (RT), or 37° C. In one embodiment, the acid is 0.5M Citric Acid pH 3.0 for one hour.

In some embodiments, the sample after acid dissociation treatment is neutralized to raise the pH with a buffer, such as PBS. In some embodiments, the sample after acid dissociation treatment is contacted with a buffer such that the sample is in an environment suitable for immune complexes to form between fluorescent-labeled anti-TNF

An illustrative description of a method for detecting and measuring the presence or level of infliximab (IFX) and/or the presence or level of autoantibodies to infliximab (ATI) in a sample is present below.

In certain aspects, the method includes a first step of acid dissociating any infliximab (IFX) bound to an autoantibody against infliximab present in the standards, controls and samples. For instances, an acid is contacted with the sample for an incubation period (e.g., room temperature for one hour). Labeled IFX (e.g., fluorescently labeled IFX such as IFX-Alexa488) and optionally, a deactivated Alexa488 loading control, is then added to in excess to compete with free IFX in the samples. The reaction is allowed to reach equilibrium.

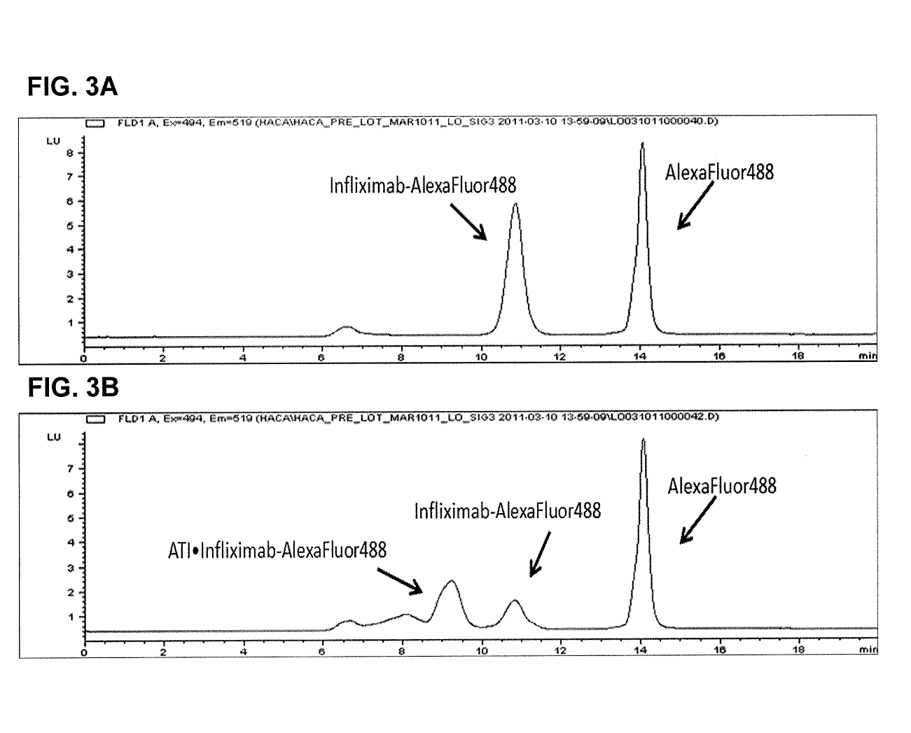

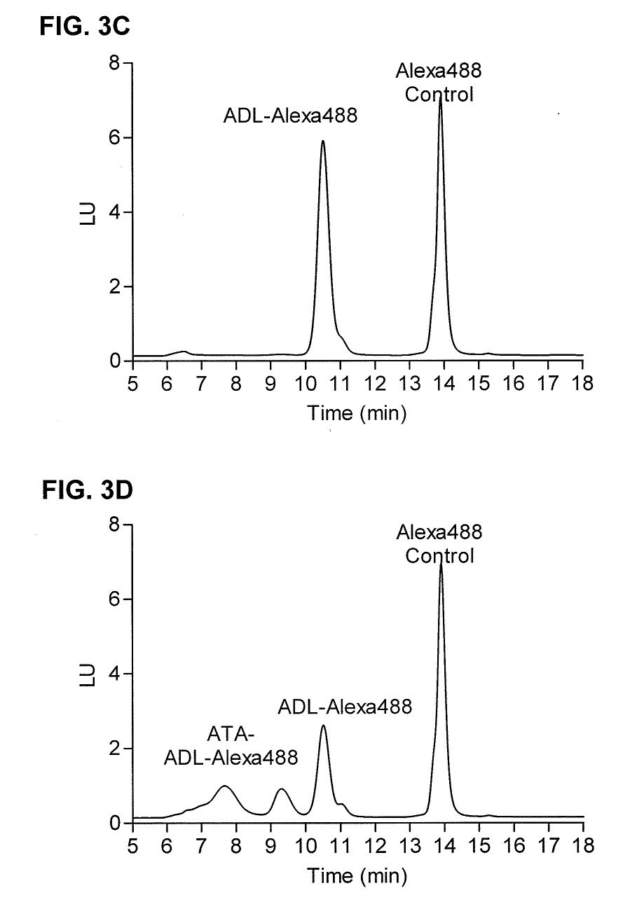

Turning now to FIG. 3A-B, complexes are formed and range in size from approximately 300 kDa for 1:1 binding to over 2000 kDa. Prior to injection, all reaction solutions (e.g., samples, standards and controls) are diluted and filtered through a filter plate. After injection and elution of the complex mixture through a column packed with gel media, free labeled anti-TNFα drug (e.g., infliximab-Alexa488 (Mw˜150 kDa, FIG. 3A)) elutes at a retention time of approximately 10-11.5 minutes while the complexes of anti-TNFα drug bound to an autoantibody against the anti-TNFα drug (e.g., ATI-Infliximab-Alexa488 complexes, FIG. 3B)) elute at the range from 6-10 minutes, and the deactivated control (e.g., Alexa488 loading control) elutes between 13.5-14.5 minutes. This real time, liquid phase assay resolves an anti-TNFα drug bound to an autoantibody against the anti-TNFα drug (e.g., ATI-Infliximab complexes) from free anti-TNFα drug (e.g., infliximab) based on the size of the complexes formed.

In some embodiments, the method of the present invention for determining the presence or level of an autoantibody to an anti-TNFα drug in a sample is performed in an automated mode. For example, in one embodiment, the automated assay comprises an automated liquid handler and an HPLC system. In some instances, the reagents, samples and other fluid components of the assay are transferred using an automated liquid handling robot, including, but not limited to, the Tecan Freedom EVO with TE-VACs, Gilson 215 or Agilent Bravo. Non-limiting examples of an HPLC system are available from Agilent Technologies (Santa Clara, Calif.), Shimadzu (Pleasanton, Calif.), and Dionex Corp. (Sunnyvale, Calif.). In some embodiments, size exclusion chromatography is performed using a gel filtration column such as aPhenomenx BioSep SEC-S3000 column or any column with a substantially similar size exclusion range.

In one embodiment, the ATI assay is performed by first acid dissociating Infliximab-ATI complexes in the standards, controls and sample. Fluorescently labeled infliximab (infliximab-Alexa488) containing an optional deactivated Alexa488 loading control is then added in excess to compete with free infliximab in the sample. A buffer is used to neutralize the reactions and all reactions are incubated for one hour to achieve equilibrium, forming various complexes of increasing molecular weight. Complexes formed range in size from approximately 300 kDa for 1:1 binding to over 2000 kDa. Prior to injection, all reaction solutions are diluted (e.g., with human serum, animal serum or BSA) and filtered through a filter plate (e.g., 0.22 μM filter plate). After injection and elution of the complex mixture through a column packed with gel media, free Infliximab-Alexa488 (M_{w}˜150 kDa) elutes at a retention time of approximately 10-11.5 minutes while ATI-Infliximab-Alexa488 complexes elute at the range from 6-10 minutes, and the optional deactivated Alexa488 loading control elutes between 13.5-14.5 minutes.

Standards and control samples for detecting ATI include, without limitation, pooled ATI positive human serum and any rabbit polyclonal antibody (e.g., whole antibody and F(ab′)2 fragment) that binds to infliximab. In some embodiments, the standards and control samples also include a diluent such as, but not limited to, normal human serum, normal rabbit serum, or BSA.

In some embodiments, a standard curve (e.g., 1.56 to 200 U/mL or 3.125 to 200 U/mL) and high (e.g., 100 U/mL or 80 U/mL), med (e.g., 50 U/mL or 20 U/mL) and low (e.g., 25 or 5 U/mL) U/mL) ATI positive controls are run during each assay.

The level of ATI is determined by the ratio of the shifted area to the free IFX peak and normalized to the internal control. Quantification of the infliximab and ATI are performed by tracking the appearance of high molecular weight peaks (Rt=6-10) and the disappearance of the free infliximab peak (Rt=10-11.5). Raw chromatograms are collected and undergo statistical analysis. The analysis includes normalizing the spectra, finding the areas under each peak, and calculating the proportion of peak area shifted to bound TNF-infliximab as a function of the total TNF/infliximab area (infliximab assay) or the proportion of peak area shifted to bound Infliximab/ATI as a function of the total infliximab/ATI area (ATI assay). With these data, standard curves are made and sample concentrations of infliximab and ATI interpolated.

The ratio of the area representing the free infliximab/Alexa488 loading control is plotted from an ATI standard curve and fit with a 5-parameter logistic model to account for asymmetry. Unknowns are calculated from a standard curve. Concentrations of ATI are reported in U/mL, wherein 100% ATI positive control serum has a concentration of 200 U/mL.

As shown in FIG. 3, quantification is performed by tracking the appearance of high molecular weight peaks (Rₜ=6-10, FIG. 3B) and/or the disappearance of the free Infliximab peak (Rₜ=10-11.5, FIG. 3A).

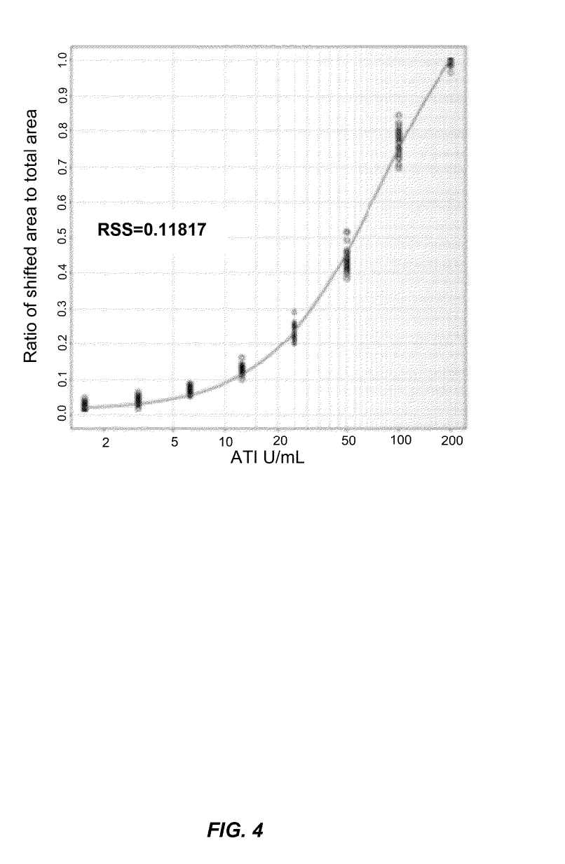

FIG. 4 shows a standard curve generated having a y-axis comprising a ratio, wherein the ratio has a numerator which is the amount of labeled complex (e.g., an anti-TNFα drug bound to an autoantibody against the anti-TNFα drug) and a denominator which is the sum of the amount of the labeled complex plus free labeled anti-TNFα drug. The x-axis comprises known amounts of the autoantibody.

The present invention also provides a method for detecting and measuring the presence or level of adalimumab (ADL) and/or the presence or level of autoantibodies to adalimumab (ATA) in a sample.

In some embodiments, the assay is performed by acid dissociation of the serum proteins in samples collected from patients treated with ADL, followed by addition of fluorescently labeled adalimumab (e.g., ADL-Alexa488) and optionally, a deactivated loading control (e.g., Alexa488). The samples are then neutralized and allowed to reach equilibrium at room temperature to form various immune complexes of increasing molecular weight. The complexes formed range in size from approximately 300 kDa for 1:1 antigen/antibody binding to over 2,000 kDa for multiple antigens/antibodies. After injection and elution of the complex mixture through a column packed with gel media (e.g., Phenomenex BioSep SEC-S3000), free ADL-Alexa488 (Mw˜150 kDa) elutes at a retention time of approximately 10-11.5 minutes while ATA-ADL-Alexa488 complexes elute at the range from 6-10 minutes and the optional deactivated Alexa488 loading control elutes between 13.5-14.5 minutes (FIG. 3C-D). The mobility shift assay of the present invention resolves ATA-adalimumab complexes from free ADL-Alexa488 based on the size of the complexes formed.

Standards and control samples for detecting ATA include, without limitation, pooled ATA positive human serum and any rabbit polyclonal antibody (e.g., whole antibody and F(ab′)2 fragment) that binds to adalimumab. In some embodiments, the standards and control samples also include a diluent such as, but not limited to, normal human serum, normal rabbit serum, or BSA.

In some embodiments, a series of standard samples are prepared by about 2-fold serial dilutions. In some instances, the standard sample that generates a complete shift for the first standard curve point and then a partial shift for the second is assigned the value of 200 U/mL.

The level of ATA is determined by the ratio of the shifted area to the free ADL peak and normalized to the internal control. Quantification can be performed by tracking the appearance of the high molecular weight peaks (Rₜ=6-10 min) or the disappearance of the free ADL-Alexa488 peak (Rₜ=10-11.5 min). Product appearance and substrate disappearance are linked by the stoichiometry of the reaction, enabling the measurement either or both concentrations. Raw chromatograms are collected and undergo statistical analysis. In some embodiments, fractions of the shifted area representing ATA-ADL-Alexa488 complexes from different concentrations of added ATA are used to generate an ATA standard curve and fitted with a 5-parameter logistic (5-PL) model to account for asymmetry.

In some embodiments, analysis of the raw chromatograms includes normalizing the data with respect to retention time by forcing the Alexa488 control peak of each spectrum to be a set time (e.g., 14 minutes). In some instances, the spectrum baseline (x-axis) of the chromatogram can be normalized in the following steps: 1) subtracting from each data point in each spectrum the luminescent unit (LU) value from the background serum sample; and 2) creating a linear model to describe the baseline using two data points at the 10^{th} and 90^{th} percentile retention times such that the baseline is as flat and as close to zero luminescent units (LU) as possible.

In some instances, a peak detection algorithm is used to find all the peaks and troughs in each spectrum per assay. In one embodiment, a cubic smoothing spline is fit to each spectrum, and peaks and troughs are defined as a change in the first derivative of the signal. A peak is a sign change of the spectrum's slope from positive to negative. Conversely, troughs are defined as a change in sign from negative to positive. For instance, the tallest peak within a window at the expected location of the free ADL-Alexa488 peak (e.g., 10 to 11 minutes) is taken to be a free peak itself. The troughs directly above and below the detected free peak define the upper and lower limits of the peak itself. In some embodiments, the bound area is comprised of several different autoantibody to anti-TNFα drug-anti-TNFα drug complexes of varying stoichiometry, such that its upper limit is defined as the lower limit of the free peak, and the bound peaks' lower limit is arbitrarily set at a low, but adjustable, retention time (e.g, about 5 minutes).

In certain instances, the areas under the bound anti-TNFα drug peak, free anti-TNFα drug peak and control peak are found by integrating the peak areas. The proportion of the anti-TNFα drug peak area shifted to bound from free is then calculated for each sample by using the formula:

p_{A}=b_{A}/(b_{A}+f_{A})

Where p_{A}=proportion of shifted area, b_{A}=area under the bound anti-TNFα drug peak, and f_{A}=the area under the free anti-TNFα drug. Optionally, the ratio of free peak area to control peak area is also calculated. The standard curve has a y-axis of p_{A}, and an x-axis of known amounts of autoantibodies against the anti-TNFα drug.

In particular embodiments, the sample is contacted with an amount of an acid that is sufficient to dissociate preformed complexes of the autoantibody and the anti-TNFα drug, such that the labeled anti-TNFα drug, the unlabeled anti-TNFα drug, and the autoantibody to the anti-TNFα drug can equilibrate and form complexes therebetween.

In preferred embodiments, the methods of the invention comprise detecting the presence or level of the autoantibody without substantial interference from the anti-TNFα drug that is also present in the sample. In such embodiments, the sample can be contacted with an amount of an acid that is sufficient to allow for the detection and/or measurement of the autoantibody in the presence of a high level of the anti-TNFα drug. In some embodiments, the phrase “high level of an anti-TNFα drug” includes drug levels of from about 10 to about 100 μg/mL, about 20 to about 80 μg/mL, about 30 to about 70 μg/mL, or about 40 to about 80 μg/mL. In other embodiments, the phrase “high level of an anti-TNFα drug” includes drug levels greater than or equal to about 10, 20, 30, 40, 50, 60, 70, 80, 90, or 100 μg/mL.

In particular embodiments, the presence of an autoantibody such as, for example, HACA is determined by comparing the level of labeled complex to a cut point (e.g., cut-off value). In certain instances, the cut point is about 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.05, 1.10, 1.15, 1.16, 1.17, 1.18, 1.19, 1.20, 1.21, 1.22, 1.23, 1.24, 1.25, 1.30, 1.35, 1.40, 1.45, 1.50, 1.55, 1.60, 1.65, 1.70, 1.75, 1.80, 1.85, 1.90, 1.95, 2.00, 2.10, 2.20, 2.30, 2.40, 2.50, 2.60, 2.70, 2.80, 2.90, 3.00, 3.50, 4.00, 4.50, 5.00, 5.50, 6.00, 6.50, 7.00, 7.50, 8.00, 8.50, 9.00, 9.50, 10.00, or more μg/ml. In certain instances, a level (concentration or amount) of labeled complex that is equal to or greater than the cut point indicates that the sample is positive for the autoantibody. In other instances, a particular cut point can be set to provide or yield a specific percentage of a clinical parameter such as sensitivity, specificity, negative predictive value, positive predictive value, overall accuracy, and combinations thereof. As a non-limiting example, the cut point for assays of the invention for detecting the presence of an anti-TNFα drug autoantibody such as HACA (i.e., ATI) can be about 1.19 μg/ml, and can provide a clinical specificity of about 97%.

In particular embodiments, the presence of an autoantibody to an anti-TNFα antibody such as infliximab (IFX) or adalimumab (ADL) is determined by comparing the level of labeled complex to a cut point (e.g., cut-off value or threshold level) established for the anti-TNFα antibody. In certain instances, the cut point is about 0.1, 0.15, 0.2, 0.25, 0.3, 0.35, 0.4, 0.45, 0.5, 0.55, 0.6, 0.65, 0.66, 0.67, 0.68, 0.69, 0.7, 0.75, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.5, 5.0, 5.5, 6.0, 6.5, 7.0, 7.5, 8.0, 8.5, 9.0, 9.5, 10.0, 15.0, or more μg/ml. In certain embodiments, a level (concentration or amount) of labeled complex that is less than the cut point indicates that the sample is positive for the autoantibody, e.g., positive for antibodies to IFX (ATI) or antibodies to ADL (ATA). In certain other embodiments, a level (concentration or amount) of labeled complex that is equal to or greater than the cut point indicates that the sample is negative for the autoantibody, e.g., negative for antibodies to IFX (ATI) or antibodies to ADL (ATA).

In some embodiments of the invention, an IFX level lower than threshold levels is associated with ATI positivity. In some embodiments, an IFX level equal to or higher than threshold is associated with ATI negativity. The threshold level of IFX is an analytical value. In some instances, the threshold level is 3 μg/ml.

C. Total Amount of Autoantibody Against the Anti-TNFα Drug

In some embodiments, the present invention provides a method to determine the total amount of autoantibody against the anti-TNFα drug in a sample. This total amount of autoantibody is the sum of autoantibody bound to unlabeled anti-TNFα drug plus the amount of autoantibody bound to labeled anti-TNFα drug. In certain instances, the autoantibody assays are performed by first acid dissociating anti-TNFα drug-autoantibody complexes in the standards, controls, samples, or a combination thereof.

As such, in one embodiment, the present invention provides a method comprising:
(a) determining the level of autoantibody bound to labeled anti-TNFα drug according to the following method:
   (i) contacting a labeled anti-TNFα drug with the sample to form a labeled complex with the autoantibody;
   (ii) subjecting the labeled complex to size exclusion chromatography to separate the labeled complex from free labeled anti-TNFα drug and to measure the amount of the labeled complex and the amount of the free labeled anti-TNFα drug;
   (iii) calculating a ratio of the amount of the labeled complex to the sum of the amount of the labeled complex plus free labeled anti-TNFα drug; and
   (iv) comparing the ratio calculated in step (iii) to a standard curve of known amounts of the autoantibody, to thereby determine the presence or level of the autoantibody, as being the amount of autoantibody bound to a labeled anti-TNFα drug; and
(b) adding the amount of autoantibody bound to unlabeled anti-TNFα drug to the level determined in step (iv) to produce the total amount of autoantibody in the sample.

In one aspect, the amount of autoantibody bound to unlabeled anti-TNFα drug is calculated by multiplying the level of autoantibody bound to labeled anti-TNFα drug of step (iv) by the amount of unlabeled anti-TNFα drug divided by the amount of labeled anti-TNFα drug.

In some aspects, the amount of unlabeled anti-TNFα drug is the weight of anti-TNFα drug. This weight can be determined by multiplying the concentration of anti-TNFα drug by the volume of serum in the assay to determine the amount of autoantibody bound to labeled anti-TNFα drug.

In some aspects, the amount of labeled anti-TNFα drug is the weight of labeled anti-TNFα drug determined by multiplying the volume of labeled anti-TNFα drug by the concentration of labeled anti-TNFα drug added to the sample.

D. Labels

An anti-TNFα drug and/or TNFα can be labeled with any of a variety of one or more detectable group(s). In preferred embodiments, an anti-TNFα drug and/or TNFα is labeled with a fluorophore or a fluorescent dye. Non-limiting examples of fluorophores or fluorescent dyes include those listed in the Molecular Probes Catalogue, which is herein incorporated by reference (see, R. Haugland, The Handbook-A Guide to Fluorescent Probes and Labeling Technologies, 10^{th} Edition, Molecular probes, Inc. (2005)). Such exemplary fluorophores or fluorescent dyes include, but are not limited to, Alexa Fluor® dyes such as Alexa Fluor® 350, Alexa Fluor® 405, Alexa Fluor® 430, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, and/or Alexa Fluor® 790, as well as other fluorophores including, but not limited to, Dansyl Chloride (DNS-Cl), 5-(iodoacetamida)fluoroscein (5-IAF), fluoroscein 5-isothiocyanate (FITC), tetramethylrhodamine 5-(and 6-)isothiocyanate (TRITC), 6-acryloyl-2-dimethylaminonaphthalene (acrylodan), 7-nitrobenzo-2-oxa-1,3,-diazol-4-yl chloride (NBD-Cl), ethidium bromide, Lucifer Yellow, 5-carboxyrhodamine 6G hydrochloride, Lissamine rhodamine B sulfonyl chloride, Texas Red™ sulfonyl chloride, BODIPY™, naphthalamine sulfonic acids (e.g., 1-anilinonaphthalene-8-sulfonic acid (ANS), 6-(p-toluidinyl)naphthalen-e-2-sulfonic acid (TNS), and the like), Anthroyl fatty acid, DPH, Parinaric acid, TMA-DPH, Fluorenyl fatty acid, fluorescein-phosphatidylethanolamine, Texas Red-phosphatidylethanolamine, Pyrenyl-phophatidylcholine, Fluorenyl-phosphotidylcholine, Merocyanine 540, 1-(3-sulfonatopropyl)-4[β-[2[(di-n-butylamino)-6 naphthyl]vinyl]pyridinium betaine (Naphtyl Styryl), 3,3′ dipropylthiadicarbocyanine (diS-C₃-(5)), 4-(p-dipentyl aminostyryl)-1-methylpyridinium (di-5-ASP), Cy-3 Iodo Acetamide, Cy-5-N-Hydroxysuccinimide, Cy-7-Isothiocyanate, rhodamine 800, IR-125, Thiazole Orange, Azure B, Nile Blue, Al Phthalocyanine, Oxaxine 1, 4′,6-diamidino-2-phenylindole (DAPI), Hoechst 33342, TOTO, Acridine Orange, Ethidium Homodimer, N(ethoxycarbonylmethyl)-6-methoxyquinolinium (MQAE), Fura-2, Calcium Green, Carboxy SNARF-6, BAPTA, coumarin, phytofluors, Coronene, metal-ligand complexes, IRDye® 700DX, IRDye® 700, IRDye® 800RS, IRDye® 800CW, IRDye® 800, Cy5, Cy5.5, Cy7, DY 676, DY680, DY682, DY780, and mixtures thereof. Additional suitable fluorophores include enzyme-cofactors; lanthanide, green fluorescent protein, yellow fluorescent protein, red fluorescent protein, or mutants and derivates thereof. In one embodiment of the invention, the second member of the specific binding pair has a detectable group attached thereto.

Typically, the fluorescent group is a fluorophore selected from the category of dyes comprising polymethines, pthalocyanines, cyanines, xanthenes, fluorenes, rhodamines, coumarins, fluoresceins and BODIPY™.

In one embodiment, the fluorescent group is a near-infrared (NIR) fluorophore that emits in the range of between about 650 to about 900 nm. Use of near infrared fluorescence technology is advantageous in biological assays as it substantially eliminates or reduces background from auto fluorescence of biosubstrates. Another benefit to the near-IR fluorescent technology is that the scattered light from the excitation source is greatly reduced since the scattering intensity is proportional to the inverse fourth power of the wavelength. Low background fluorescence and low scattering result in a high signal to noise ratio, which is essential for highly sensitive detection. Furthermore, the optically transparent window in the near-IR region (650 nm to 900 nm) in biological tissue makes NIR fluorescence a valuable technology for in vivo imaging and subcellular detection applications that require the transmission of light through biological components. Within aspects of this embodiment, the fluorescent group is preferably selected form the group consisting of IRDye® 700DX, IRDye° 700, IRDye® 800RS, IRDye® 800CW, IRDye® 800, Alexa Fluor® 660, Alexa Fluor® 680, Alexa Fluor® 700, Alexa Fluor® 750, Alexa Fluor® 790, Cy5, Cy5.5, Cy7, DY 676, DY680, DY682, and DY780. In certain embodiments, the near infrared group is IRDye® 800CW, IRDye® 800, IRDye® 700DX, IRDye® 700, or Dynomic DY676.

Fluorescent labeling is accomplished using a chemically reactive derivative of a fluorophore. Common reactive groups include amine reactive isothiocyanate derivatives such as FITC and TRITC (derivatives of fluorescein and rhodamine), amine reactive succinimidyl esters such as NHS-fluorescein, and sulfhydryl reactive maleimide activated fluors such as fluorescein-5-maleimide, many of which are commercially available. Reaction of any of these reactive dyes with an anti-TNFα drug results in a stable covalent bond formed between a fluorophore and an anti-TNFα drug.

In certain instances, following a fluorescent labeling reaction, it is often necessary to remove any nonreacted fluorophore from the labeled target molecule. This is often accomplished by size exclusion chromatography, taking advantage of the size difference between fluorophore and labeled protein.

Reactive fluorescent dyes are available from many sources. They can be obtained with different reactive groups for attachment to various functional groups within the target molecule. They are also available in labeling kits that contain all the components to carry out a labeling reaction. In one preferred aspect, Alexa Fluor® 647 C2 maleimide is used from Invitrogen (Cat. No. A-20347).

Specific immunological binding of an anti-drug antibody (ADA) to an anti-TNFα drug can be detected directly or indirectly. Direct labels include fluorescent or luminescent tags, metals, dyes, radionuclides, and the like, attached to the antibody. In certain instances, an anti-TNFα drug that is labeled with iodine-125 (¹²⁵I) can be used for determining the concentration levels of ADA in a sample. In other instances, a chemiluminescence assay using a chemiluminescent anti-TNFα drug that is specific for ADA in a sample is suitable for sensitive, non-radioactive detection of ADA concentration levels. In particular instances, an anti-TNFα drug that is labeled with a fluorochrome is also suitable for determining the concentration levels of ADA in a sample. Examples of fluorochromes include, without limitation, Alexa Fluor® dyes, DAPI, fluorescein, Hoechst 33258, R-phycocyanin, B-phycoerythrin, R-phycoerythrin, rhodamine, Texas red, and lissamine. Secondary antibodies linked to fluorochromes can be obtained commercially, e.g., goat F(ab′)₂ anti-human IgG-FITC is available from Tago Immunologicals (Burlingame, Calif.).

Indirect labels include various enzymes well-known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.). A useful secondary antibody linked to an enzyme can be obtained from a number of commercial sources, e.g., goat F(ab′)₂ anti-human IgG-alkaline phosphatase can be purchased from Jackson ImmunoResearch (West Grove, Pa.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis of ADA levels can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

In certain embodiments, size exclusion chromatography is used. The underlying principle of SEC is that particles of different sizes will elute (filter) through a stationary phase at different rates. This results in the separation of a solution of particles based on size. Provided that all the particles are loaded simultaneously or near simultaneously, particles of the same size elute together. Each size exclusion column has a range of molecular weights that can be separated. The exclusion limit defines the molecular weight at the upper end of this range and is where molecules are too large to be trapped in the stationary phase. The permeation limit defines the molecular weight at the lower end of the range of separation and is where molecules of a small enough size can penetrate into the pores of the stationary phase completely and all molecules below this molecular mass are so small that they elute as a single band.

In certain aspects, the eluent is collected in constant volumes, or fractions. The more similar the particles are in size, the more likely they will be in the same fraction and not detected separately. Preferably, the collected fractions are examined by spectroscopic techniques to determine the concentration of the particles eluted. Typically, the spectroscopy detection techniques useful in the present invention include, but are not limited to, fluorometry, refractive index (RI), and ultraviolet (UV). In certain instances, the elution volume decreases roughly linearly with the logarithm of the molecular hydrodynamic volume (i.e., heaver moieties come off first).

The present invention further provides a kit for detecting the presence or level of an autoantibody to an anti-TNFα drug in a sample. In particular embodiments, the kit comprises one or more of the following components: an acid (or mixture of acids), a labeled anti-TNFα drug (e.g., a labeled anti-TNFα antibody), a labeled internal control, a neutralizing agent (or mixtures thereof), means for detection (e.g., a fluorescence detector), a size exclusion-high performance liquid chromatography (SE-HPLC) instrument, and/or instructions for using the kit.

### III. Examples

### Example 1

### Mobility Shift Assay for Anti-TNF-α Drug Infliximab

This example illustrates one embodiment of the method described herein for determining the presence of infliximab in a sample. The assay is performed by incubating fluorescently labeled recombinant TNF-α (TNF-Alexa488) containing a deactivated Alexa488 loading control with sera containing infliximab and allowed to reach equilibrium, forming various complexes of increasing molecular weight. Complexes are formed ranging in size from approximately 200 kDa for 1:1 binding to over 2000 kDa. After injection and elution of the complex mixture through a column packed with gel media, free TNF-Alexa488 (Mw˜51 kDa) elutes at a retention time of approximately 11-12.5 minutes while infliximab-TNF-Alexa488 complexes elute at the range from 6-10 minutes, and the deactivated Alexa488 loading control elutes between 13.5-14.5 minutes. This real time, liquid phase assay resolves infliximab-TNF complexes from free TNF based on the size of the complexes formed.

Quantification can be performed by tracking the appearance of high molecular weight peaks (Rₜ=6-10 min) or the disappearance of the free labeled TNF peak (Rₜ=11-12.5 min). FIG. 2 shows an exemplary standard curve. The y-axis comprises a ratio, wherein the ratio has a numerator which is labeled complex (e.g., labeled TNFα bound to an anti-TNFα drug) and a denominator which is the sum of the labeled complex plus free labeled TNFα. The x-axis comprises known amounts of the anti-TNFα drug. Unknowns are determined from the standard curve and the effective concentration of infliximab in 100% serum calculated by multiplying the result by the dilution factor.

The infliximab standard curve and positive controls were prepared by diluting infliximab in normal human serum. The standard curve (0.73 to 46.88 μg/mL) and High (15.63 μg/mL), Medium (7.81 μg/mL) and Low (3.91 μg/mL) Infliximab Positive Controls (IPC) were run during each assay. The reference range of the assay was less than 1.0 μg/mL. The reportable range was 1.0-34.0 μg/mL. Sample values greater than 34.0 μg/mL were reported as >34.0 μg/mL. Sample values lower than μg/mL were reported as <1.0 μg/mL.

Data analysis is done in an automated manner using a computer program. The program normalizes the spectra, finds the areas under each peak, and calculates the proportion of peak area shifted to bound TNF-infliximab as a function of the total TNF/infliximab area. With these data, standard curves are made and sample concentrations of infliximab interpolated.

### Example 2

### Mobility Shift Assay for Autoantibodies Against Anti-TNF-α Drug Infliximab (ATI)

This example illustrates one embodiment of the method described herein for determining the total amount of autoantibody against infliximab present in a sample. The assay was performed by first acid dissociating infliximab-ATI complexes in the standards, controls and patient serum samples with 0.5 M Citric Acid pH 3.0 with an one hour incubation. Fluorescently labeled infliximab (infliximab-Alexa488) containing a deactivated Alexa488 loading control was then added in excess to compete with free infliximab in the samples. 10×PBS was used to neutralize the reactions and all reactions were incubated for one hour to achieve equilibrium, forming various complexes of increasing molecular weight. Complexes formed range in size from approximately 300 kDa for 1:1 binding to over 2000 kDa. Prior to injection, all reaction solutions were diluted to 2% serum and filtered through a 0.22 μM filter plate. After injection and elution of the complex mixture through a column packed with gel media, free infliximab-Alexa488 (M_{w}˜150 kDa) eluted at a retention time of approximately 10-11.5 minutes while ATI-infliximab-Alexa488 complexes eluted at the range from 6-10 minutes, and the deactivated Alexa488 loading control eluted between 13.5-14.5 minutes. The method described herein resolved ATI-infliximab complexes from free infliximab based on the size of the complexes formed.

Quantification was performed by tracking the appearance of high molecular weight peaks (Rₜ=6-10) and the disappearance of the free infliximab peak (Rₜ=10-11.5). FIG. 4 shows an exemplary standard curve. The y-axis comprises a ratio, wherein the ratio has a numerator which is the amount of labeled complex (e.g., an anti-TNFα drug bound to an autoantibody against the anti-TNFα drug) and a denominator which is the sum of the amount of the labeled complex plus free labeled anti-TNFα drug. The x-axis comprises known amounts of the autoantibody. Unknowns are calculated from the standard curve. Concentrations of ATI are reported in arbitrary U/mL, 100% ATI Positive Control serum has a concentration of 200 U/mL.

The Residual Sum of Squares (RSS) of the standard curve was determined to judge the quality of the fit. If the RSS was >0.01 (e.g., representing a poor fit), the starting parameters were loosened and a fit was attempted again. If the RSS was still >0.01, the standard with the lowest shifted area was removed, and the statistical analysis were repeated once if RSS>0.01. If the curve adaptation fails]ed once more, wherein RSS>0.01, then the analysis was aborted.

The ATI standard curve and positive controls were prepared by diluting pooled positive serum in normal human serum. The standard curve (1.56 to 100 U/mL) and High (100 U/mL, Med (50 U/mL) and Low (25 U/mL) ATI Positive Controls (APC) were run during each assay. The reference range of the assay was less than 3.1 U/mL. The reportable range was 3.1-100 U/mL. Sample values greater than 100 U/mL were reported as >100 U/mL. Sample values lower than 3.1 U/mL were reported as <3.1 U/mL.

Data analysis was performed in an automated manner using the statistically analysis program R. The analysis normalized the spectra, found the areas under each peak, and calculated the proportion of peak area shifted to bound Infliximab/ATI as a function of the total Infliximab/ATI area. With these data, standard curves were made and sample concentrations of ATI interpolated.

### Example 3

### Calculation of Total Amount of Autoantibody to Infliximab (Total ATI)

This example describes methods of calculating the total amount of autoantibody against infliximab in a sample from a patient.

In this illustrative example, in order to calculate the amount of total autoantibody, the following equation is used:

Total ATI=ATI bound to unlabeled IFX+ATI bound to labeled IFX

### (a) Calculation of ATI Bound to Unlabeled Infliximab

Using the equilibrium equation A+B+C=AC+BC, where A=unlabeled Infliximab, B=Labeled-infliximab and C=ATI, the total amount of ATI present in the serum can be accurately calculated.

For this equation the following values are known for each sample:

A is the concentration calculated from testing with the infliximab mobility shift assay.

B is the known amount of infliximab-AlexaFluor488 spiked into the sample.

BC is the concentration calculated from the ATI mobility shift assay.

Knowing that the sample is acid dissociated and then allowed to reach equilibrium:

$$\frac{BC}{B} = \frac{A C}{A}$$

By solving for AC, the concentration of ATI bound to unlabeled infliximab is obtained. The total ATI in the sample then is equal to AC+BC.

$$\begin{matrix}ATI bound to \\ unlabeled IFX\end{matrix} = \frac{\begin{matrix}U \text{/} mL ATI from \\ mobility shift \\ assay\end{matrix} \times \begin{matrix}mg \\ unlabeled \\ IFX\end{matrix}}{mg labeled IFX}$$

The detailed equation for calculation of ATI bound to unlabeled IFX is as follows:

$$\begin{matrix}ATI bound \\ to \\ unlabeled \\ IFX\end{matrix} = \frac{\begin{matrix}U \text{/} mL ATI \\ from mobility \\ shift assay\end{matrix} \times \begin{matrix}µg \text{/} mL IFX \\ in 100 \% \\ Serum\end{matrix} \times \begin{matrix}Vol of 100 \% serum \\ added to ATI Assay\end{matrix}}{\begin{matrix}Volume of \\ labeled IFX \\ added to sample\end{matrix} \times \begin{matrix}Concentration of \\ labeled IFX \\ added to sample\end{matrix}}$$

### (b) Calculation of Total ATI in Patient Samples

The total concentration of ATI in a patient sample is calculated in the following manner:
The amount of ATI bound to labeled IFX is determined from the ATI mobility shift assay.
If the measurement of ATI bound to labeled IFX is between the limits of quantification, the ATI mobility shift assay result is added to the calculated concentration of antibody bound to intrinsic (unlabeled) IFX to produce the Total ATI concentration.

Total ATI=ATI bound to unlabeled IFX+ATI bound to labeled IFX
The concentration of ATI bound to unlabeled IFX is calculated by multiplying the concentration of ATI bound to labeled IFX by the measured concentration of serum IFX and by the inverse of the concentration of labeled IFX of the sample used to measure concentration of ATI bound to labeled IFX.

$$\begin{matrix}ATI bound to \\ unlabeled IFX\end{matrix} = \frac{\begin{matrix}U \text{/} mL ATI from \\ mobility shift assay\end{matrix} \times \begin{matrix}µg unlabeled \\ IFX\end{matrix}}{µg labeled IFX}$$

### (c) Exemplary Calculation of Total ATI

Example of the calculation for a patient sample with the following mobility shift assay results:

ATI=25 U/mL

Infliximab=1 μg/mL (0.001 mg/mL in the equation below)

$$\begin{matrix}ATI bound to \\ unlabeled \\ IFX\end{matrix} = \frac{\begin{matrix}25 U \text{/} mL ATI \times \\ 0.001 mg \text{/} mL IFX \times \\ 0.024 mL serum in ATI assay\end{matrix}}{\begin{matrix}0.033 mL of labeled IFX \times \\ 0.0135 mg \text{/} mL of labeled IFX\end{matrix}}$$

ATI bound to Unlabeled IFX=1.3 U/mL

Total ATI=25 U/mL+1.3 U/mL

Total ATI=26.3 U/mL

(d) Calculation of Total Amount of Autoantibodies (Total ATI) from Automated Mobility Shift Assay

Total ATI was calculated by the following equations:

Partial ATI(U/mL)=ATI Assay Result;

wherein the level of ATI in the sample determined by the mobility shift assay as described herein, e.g., Example 2.

Unbound ATI(U/mL)=(IFX Assay Result)×(ATI Assay Result)×(0.05387);

wherein IFX Assay Result represents the level of IFX in a sample determined by the mobility shift assay as described herein, e.g., Example 1.

Total ATI(U/mL)=Partial ATI(U/mL)+Unbound ATI

### (e) Exemplary Calculation of Total ATI Automated Mobility Shift Assay

Example of the calculation for a patient sample with the following mobility shift assay results:

### ATI=25 U/mL

Infliximab=1 μg/mL

### Unbound ATI=1×25×0.05387=1.34675 U/mL

### Total ATI=26.34675 U/mL

### Example 4

### Automated Mobility Shift Assays for Infliximab and ATI

This example shows that automated mobility shift assays for infliximab and total ATI, as described herein, can be used an alternative to manual assays described herein. Thus, automated assays can be used for infliximab and total ATI determination.

Regression plots were prepared from the mean values for manual vs. automated assay results. Linear regression analysis of manual vs. automated means for both infliximab and total ATI assays showed slopes of 1.086 and 0.9655, respectively. The r² values 0.9796 and 0.9913 meet the acceptance criteria of r^{2≧}>0.95. These results demonstrate acceptable response ranges between the two assay formats.

Individual automated assay duplicate values were plotted against the mean manual assay value for each sample. Linear regression analysis of manual means vs. automated duplicates for both infliximab and total ATI assays showed slopes of 1.077 and 0.9658, respectively. The r² values were 0.9703 and 0.9897, respectively. These results also demonstrate acceptable response ranges between the two assay formats.

For an analysis of bias, the difference between the mean manual and automated assay values were plotted against the average of the mean manual and automated assay values for each sample. The horizontal centerline of this plot has the value of zero. Plots for the infliximab and the total ATI assays are produced and summarized in the Table 1.

TABLE 1
Summary of IFX and total ATI assays.

| | IFX Difference vs. | Total ATI Difference vs. |
|---|---|---|
| | Average | Average |
| | (Bland-Altman Analysis) | (Bland-Altman Analysis) |
| Bias | 0.29 | −2.9 |
| SD of bias | 1.2 | 4.7 |
| 95% Limits of | | |
| Agreement | | |
| From | −2.1 | −12 |
| To | 2.7 | 6.3 |

In a comparison of the manual and automated infliximab assay values, the bias was 0.29±1.2. The standard deviation (SD) of the bias was used to calculate the limits of agreement. 95% of assay values were predicted to fall between the upper and lower limits of agreement 2.7 and −2.1, respectively. In a comparison of the manual and automated total ATI assay values, the bias was −2.9±4.7.95% of values were predicted to fall between the upper and lower limits of agreement 6.3 and −12, respectively. Analysis revealed that the bias for the manual Infliximab and ATI assays meets the acceptance criteria of ±15%.

In summary. automated mobility shift assays for infliximab and ATI met the acceptance criteria for equivalence to the manual assays for infliximab and ATI, respectively. The automated assays as described herein are an acceptable methods for infliximab and Total ATI determination.

### Example 5

### Mobility Shift Assay for Autoantibodies Against ADL (ATA)

This example illustrates one embodiment of the method described herein for measuring the total amount of ATA present in a sample. First, acid dissociation of the serum proteins was performed on a sample collected from a patient treated with Adalimumab. The sample was contacted with citric acid, pH 3.0 and incubated for one hour at room temperature to free the ATA in the sample from other bound proteins. Next, the sample was contacted with fluorescently labeled Adalimumab (ADL-Alexa488) and a deactivated Alexa488 loading control, and the pH of the sample was neutralized with a 10×PBS solution (pH 7.3). The sample in contact with ADL-Alexa488 and the Alexa488 loading control was incubated at room temperature for one hour. The incubated sample was diluted with BSA and filtered through a 0.22 μM filter membrane before HPLC analysis on a column packed with gel media (Phenomenex BioSep SEC-S3000).

HPLC analysis was performed by running the standards, the high, medium, and low controls and then the processed patient sample. Free ADL-Alexa488 (M_{w}˜150 kDa) eluted at a retention time of approximately 10-11.5 minutes, ATA-ADL-Alexa488 complexes eluted at the range from 6-10 minutes and the deactivated Alexa488 loading control eluted between 13.5-14.5 minutes (FIG. 3C,D).

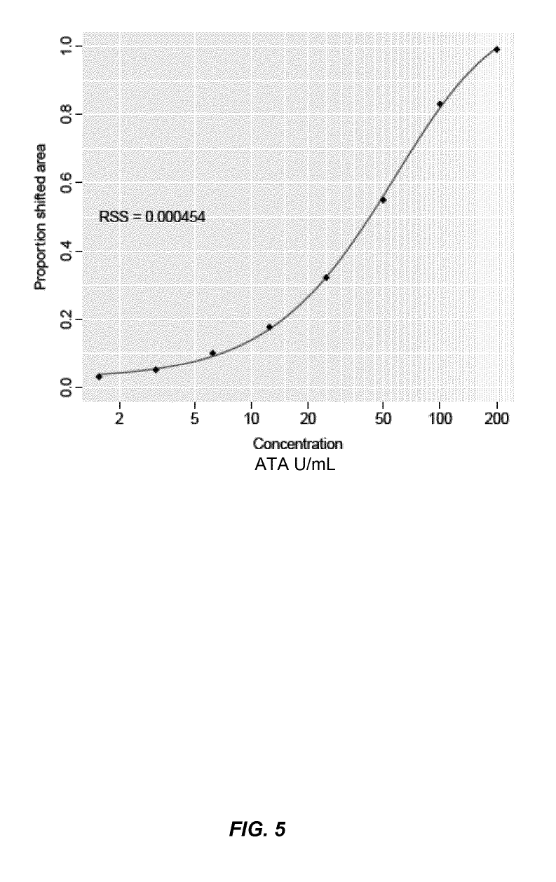

Quantification of ATA and free ADL was performed by tracking the appearance of the high molecular weight peaks (Rₜ=6-10 min) or the disappearance of the free ADL-Alexa488 peak (Rₜ=10-11.5 min). Raw chromatograms were collected in Agilent ChemStation and then exported to the program “R” for automated analysis. Fractions of the shifted area representing ATA-ADL-Alexa488 complexes from different concentrations of added ATA were used to generate an ATA standard curve and fitted with a 5-parameter logistic (5-PL) model to account for asymmetry. Unknowns were determined from the standard curve and given the effective concentration of ATA in 100% serum. FIG. 5 shows an exemplary standard curve for ATA.

### Example 6

### Calculation of Total Amount of Autoantibody to Adalimumab (Total ATA)

When ADL is present in a sample, the total ATA is calculated using the equilibrium equation:

A+B+C=AC+BC,

where A=unlabeled adalimumab, B=labeled-adalimumab and C=ATA

In this equation the following values are known for each sample:

A is the concentration from performing the adalimumab mobility shift assay.

B is the known amount of adalimumab-AlexaFluor488 spiked into the sample.

BC is the concentration determined from the ATA mobility shift assay.

Knowing that the sample is acid dissociated and then allowed to reach equilibrium:

$$\frac{BC}{B} = \frac{A C}{A}$$

By solving for AC, the concentration of ATA bound to unlabeled adalimumab is obtained.

Therefore, the total ATA in the sample is then equal to AC+BC.

$$\begin{matrix}ATA bound to \\ unlabeled IFX\end{matrix} = \frac{\begin{matrix}U \text{/} mL ATA from \\ mobility shift \\ assay\end{matrix} \times \begin{matrix}µg \\ Unlabeled \\ ADL\end{matrix}}{µg Labeled ADL}$$

(a) Calculation of ATI bound to unlabeled ADL

Detailed equation for calculation of ATA bound to unlabeled ADL:

$$\begin{matrix}ATA bound \\ to \\ unlabeled \\ IFX\end{matrix} = \frac{\begin{matrix}U \text{/} mL ATA \\ from mobility \\ shift assay\end{matrix} \times \begin{matrix}µg \text{/} mL ADL \\ in 100 \% \\ serum\end{matrix} \times \begin{matrix}Vol of 100 \% \\ serum added \\ to ATA assay\end{matrix}}{\begin{matrix}Volume of labeled \\ ADL added to sample\end{matrix} \times \begin{matrix}Concentration of \\ labeled ADL added \\ to sample\end{matrix}}$$

Exemplary calculation of ATA bound to unlabeled ADL and total ATA for a sample with 100 ug/mL ADL and an ATA mobility shift assay result of 4.2 U/mL ATA.

$$\begin{matrix}ATA bound \\ to \\ unlabeled \\ ADL\end{matrix} = \frac{4.20 U \text{/} mL \times 0.1 mg \text{/} mL \times 0.024 mL}{0.033 mL \times 0.0135 mg \text{/} mL}$$

$$\begin{matrix}ATA bound to \\ unlabeled ADL\end{matrix} = 22.6 U \text{/} mL$$

### (b) Calculation of Total Amount of Autoantibodies (Total ATA)

Calculation of total ATA:

Total ATA=ATA bound to unlabeled ADL+ATA bound to labeled ADL

Exemplary calculation of total ATA:

$$\begin{matrix}Total ATA = 22.6 U \text{/} mL + 4.2 U \text{/} mL \\ = 26.8 U \text{/} mL\end{matrix}$$

### Example 7

### Development and Validation of a Homogeneous Mobility Shift Assay for the Measurement of Infliximab and Antibodies-to-Infliximab Levels in Patient Serum

### Abstract

Antibody-based drugs such as infliximab (IFX) are effective for the treatment of inflammatory bowel disease (IBD) and other immune-mediated disorders. The development of antibodies against these drugs may result in unfavorable consequences, including the loss of drug efficacy, hypersensitivity reactions, and other adverse events. Therefore, accurate monitoring of serum drug and anti-drug antibody levels should be an important part of therapy for patients being treated with an antibody-based drug. Current methods for the assessment of anti-drug antibodies and drug levels, involving various bridging ELISA and radioimmunoassay techniques, are limited by their sensitivity, interference, and/or complexity. To overcome these limitations, we have developed a non-radiolabeled homogeneous mobility shift assay (HMSA) to measure the antibodies-to-infliximab (ATI) and IFX levels in serum samples. Full method validation was performed on both the ATI- and IFX-HMSA, and the clinical sample test results were also compared with those obtained from a bridging ELISA method to evaluate the difference in performance between the two assays. Validation of the ATI-HMSA revealed a lower limit of quantitation of 0.012 μg/mL in serum. The linear range of quantitation was 0.029-0.54 μg/mL. The intra- and inter-assay precision was less than 20% of coefficient of variation (CV), and the accuracy (% error) of the assay was less than 20%. In serum samples, ATI as low as 0.036 μg/mL can be measured, even in the presence of 60 μg/mL of IFX in the serum. Sera from 100 healthy subjects were tested to determine the cut point of the assay. ATI-positive samples that had been previously analyzed by using a bridging ELISA from 100 patients were also measured by the new method. There was a high correlation between the two methods for ATI levels (p<0.001). Significantly, the new method identified five false-positive samples from the bridging ELISA method. Validation of the mobility shift IFX assay also showed high assay sensitivity, precision and accuracy. The HMSA method may also be applied to other protein-based drugs to accurately detect serum drug and anti-drug antibody levels.

### 1. Introduction

Tumor necrosis factor-alpha (TNF-α) plays a pivotal role in the pathogenesis of inflammatory bowel disease (IBD), rheumatoid arthritis (RA), and other autoimmune disorders (Suryaprasad et al., The biology of TNF blockade, Autoimmun. Rev 2, 346 (2003).) Protein-based drugs that block TNF-α such as infliximab (a human-murine chimeric monoclonal IgG1κ) or adalimumab (a fully human monoclonal antibody) are effective in reducing disease activity of these inflammatory disorders (Tracey et al., Tumor necrosis factor antagonist mechanisms of action: a comprehensive review, Pharmacol Ther. 117, 244 (2008)). However, over 30% of patients fail to respond to anti-TNF-α therapy, and many who initially respond later require higher or more frequent dosing due to a failure to maintain the initial response, especially in the IBD patient population (Hanauer et al., Maintenance infliximab for Crohn's disease: the ACCENT I randomised trial, Lancet 359, 1541 (2002); Gisbert et al., Loss of response and requirement of infliximab dose intensification in Crohn's disease: a review, Am J Gastroenterol 104, 760 (2009); Regueiro et al., Infliximab dose intensification in Crohn's disease, Inflamm Bowel Dis 13, 1093 (2007)). There is now compelling evidence that demonstrates that the loss of response in these patients is a result of a failure to achieve and maintain adequate therapeutic drug levels in blood and/or from the formation of anti-drug antibodies (Miheller et al., Anti-TNF trough levels and detection of antibodies to anti-TNF in inflammatory bowel disease: are they ready for everyday clinical use? Expert Opin. Biol Ther. 12, 179 (2012)). Anti-drug antibodies could cause adverse events such as serum sickness and hypersensitivity reactions (Brennan et al., Safety and immunotoxicity assessment of immunomodulatory monoclonal antibodies, MAbs. 2, 233 (2010); Emi et al., Immunogenicity of Anti-TNF-alpha agents in autoimmune diseases, Clin Rev Allergy Immunol 38, 82 (2010)), and it is hypothesized that their formation may also increase drug clearance and/or neutralize the drug effect, thereby potentially contributing to the loss of response. Moreover, recent data suggest that the standard dosing regimen for TNF-α-blocking drugs may be suboptimal in some IBD patients, and an individualized dosing regimen to achieve therapeutic drug levels may be important to maximize the initial drug response and to maintain remission (Colombel et al., Therapeutic drug monitoring of biologics for inflammatory bowel disease, Inflamm Bowel Dis 18, 349 (2012)). Therefore, accurate monitoring of serum drug and anti-drug antibody levels should be an important part of therapy for patients being treated with protein-based drugs. While monitoring for serum drug levels and for the formation of anti-drug antibodies are routine components of early drug development and are mandatory during clinical trials (Shankar et al., Scientific and regulatory considerations on the immunogenicity of biologics, Trends Biotechnol, 24, 274 (2006)), these activities have generally not been adopted in clinical practice. This deficiency may be partially explained by technical issues of the available monitoring assays, which limit their utility as part of routine clinical practice.

Current methods for the assessment of anti-drug antibodies and drug levels in serum mainly utilize the bridging ELISA method (Baert et al., Influence of immunogenicity on the long-term efficacy of infliximab in Crohn's disease, N. Engl. J Med 348, 601 (2003)) and, occasionally, the radioimmunoassay (RIA) method (Aarden et al., Immunogenicity of anti-tumor necrosis factor antibodies-toward improved methods of anti-antibody measurement. Curr. Opin. Immunol. 20, 431 (2008)). However, a major limitation of the bridging ELISA methods in measuring anti-drug antibody levels is the inability to accurately detect the antibodies in the presence of the drug in circulation due to cross-interference. Specifically, the circulating drug would interfere with the capture of anti-drug antibodies by the same drug initially coated on the ELISA plate, thus limiting the ELISA's ability to detect anti-drug antibodies and resulting in a lower sensitivity for detection in the presence of IFX. Therefore, ELISA methods can only measure anti-drug antibodies accurately when there is no drug in circulation, which significantly limits its clinical utility. The disadvantages of the RIA method are associated with the complexity and safety concerns related to the handling of radioactive material as well as the prolonged incubation time needed to reach equilibrium for proper measurements. Therefore, there is a large unmet medical need to develop a simple and accurate assay that can overcome these limitations and provide clinicians with valuable quantitative measurements that they can then use to optimize the management of patients on biologic therapies. Here, we have developed and validated a novel homogenous mobility-shift assay (HMSA) using size-exclusion high-performance liquid chromatography (SE-HPLC) to quantitatively measure both induced antibodies-to-infliximab (ATI) levels and IFX levels in serum samples collected from IBD patients being treated with IFX.

### 2. Materials and Methods

### 2.1. Materials

Individual serum samples from healthy controls were obtained from blood bank donors (Golden West Biologics, Temecula, Calif.). Sera from IBD patients treated with IFX were drawn according to a protocol approved by an Institutional Review Board (IRB)/Ethics Committee. Unless otherwise noted, all reagents and chemicals were obtained from either Thermo Fisher Scientific (Waltham, Mass.) or Sigma Aldrich Corporation (St. Louis, Mo.).

### 2.2. Conjugation of IFX and TNF-α

Commercially-available infliximab (Remicade™, Janssen Biotech, Inc., Horsham, Pa.) was buffer exchanged with phosphate buffered saline (PBS, pH 7.3) and labeled with AlexaFluor 488 (Life Technology, Carlsbad, Calif.) following the manufacturer's instructions. Briefly, a reaction mixture consisting of 10 mg of IFX, 154 μg of AlexaFluor 488 dye, and 1 mL 1×PBS (pH 8.0) was incubated in the dark at room temperature (RT) for 1 hour with constant stirring. A desalting column was then used to remove free AlexaFluor 488, and the infliximab-AlexaFluor 488 conjugate (IFX-488) was collected. The protein concentration and labeling efficiency of the conjugate was measured by using a NanoDrop Spectrophotometer (Thermo Fisher Scientific, Waltham, Mass.). Only those conjugates containing 2 to 3 fluorescent dyes per antibody qualified for the ATI-HMSA.

The procedure for the labeling of recombinant TNF-α (RayBiotech, Inc, Norcross, Ga.) with AlexaFluor 488 was identical to that used for the labeling of IFX. The molar ratio of TNF-α to fluorescent dye in the reaction mixture was 1:6 and the resulting TNF-α-AlexaFluor 488 conjugate (TNF-488) contained 1-2 dye molecules per TNF-α.

### 2.3. Internal Control (IC) for the HPLC Analysis and Preparation of IFX-488/IC and TNF-488/IC

Activated AlexaFluor 488 (1 mg) and 4 mL 1M Tris buffer (pH 8.0) were mixed for 1 hour on a magnetic stirrer at RT to block the active site on the dye. The resulting solution was buffer-exchanged with 1×PBS. The blocked AlexaFluor 488 was used as the IC and combined with IFX-488 and TNF-488, respectively, at a molar ratio of 1:1. The resultant IFX-488/IC and TNF-488/IC were used to normalize the labeled IFX and TNF-α in the reaction mixture used for HPLC analysis. The amount of IFX-488/IC and TNF-488/IC employed for the HPLC analysis was based on the IFX-488 and TNF-488 concentrations only.

### 2.4. Preparation of Calibration Standards and Quality Control Samples

ATI-positive sera was prepared by pooling individual patient serum samples identified as containing high concentrations of ATI and was negative for IFX as measured by a bridging ELISA method (Baert et al., Influence of immunogenicity on the long-term efficacy of infliximab in Crohn's disease, N. Engl. J Med 348, 601 (2003)). The relative amount of ATI in the pooled serum was estimated by comparing the fluorescent intensity of the ATI-IFX488 immune complex in SE-HPLC with a known concentration of IFX-488. The pooled ATI calibration serum was aliquoted and stored at −70° C. To generate a standard curve, one aliquot of the stock ATI calibration serum was thawed and diluted to 2% with normal human serum (NHS) in HPLC assay buffer (1×PBS, pH 7.3) to concentrations of 0.006, 0.011, 0.023, 0.045, 0.090, 0.180, 0.360, and 0.720 μg/mL. Three quality control (QC) samples were prepared by diluting the calibration serum in assay buffer with 0.1% BSA to yield the high (0.36 μg/mL), mid (0.18 μg/mL), and low (0.09 μg/mL) control concentrations. Similarly, IFX calibration standards were prepared by serially diluting a stock solution of 93.75 μg/mL in 100% NHS. After serial dilution, each standard was added to the assay plate and diluted with assay buffer containing 0.1% BSA to yield concentrations of 0.03, 0.06, 0.12, 0.23, 0.47, 0.94, 1.88 and 3.75 μg/mL with 4% NHS. Three IFX QC samples were prepared by diluting the IFX calibration standard with assay buffer and 0.1% BSA to yield the high (0.63 μg/mL), mid (0.31 μg/mL) and low (0.16 μg/mL) control concentrations.

### 2.5. Assay Procedures

### 2.5.1. ATI Homogenous Mobility Shift Assay (ATI-HMSA)

The assay was prepared in a 96-well plate format. In order to reduce interference from circulating drug, an acid dissociation step was employed. Briefly, a solution containing a 24 μL aliquot of serum sample, 5.5 μL 0.5 M citric acid (pH 3.0), and 10.9 μL HPLC grade water were added to each well and incubated for one hour at RT to free the ATI in the patient serum samples from other bound proteins. Following the acid dissociation step, 6 μL of a 74 μg/mL IFX-488/IC solution was added and the reaction mixture was immediately neutralized with 27.6 μL of 10×PBS (pH 7.3). The plate was incubated for another hour at RT on an orbital shaker to complete the formation of the immune complexes. The incubated serum samples were then diluted to a final serum concentration of 2% by pipetting 18.4 μL of each sample solution, 22.6 μL 10×PBS (pH 7.3), and 259 μL HPLC grade water into the wells of a new 96-well plate. In this plate, the first four wells contained, respectively: 300 μL each of HPLC buffer as a blank, aqueous SECT column standard (Phenomenex, Torrance, Calif.) to monitor the resolution of the HPLC column, acid-dissociated 2% NHS, and acid-dissociated 2% NHS with 110 ng IFX-488/IC for calibrating the HPLC system. The next eight wells contained 300 μL each of the ATI calibration standards (0.006, 0.011, 0.023, 0.045, 0.090, 0.180, 0.360, and 0.720 μg/mL) with 110 ng IFX-488/IC for generating the standard curve. The next nine wells contained, respectively, 300 μL each of the three QC controls (high, mid and low) in triplicate with 110 ng IFX-488/IC to establish the precision and accuracy of the assay. The remaining wells were then filled with 300 μL of the prepared patient serum samples. After mixing on an orbital shaker for 1 min at RT, the samples were filtered through a MultiScreen-Mesh Filter plate equipped with a Durapore membrane (0.22 μm; EMD Millipore, Billerica, Mass.) into a 96-well receiver plate (Nunc, Thermo Fisher Scientific, Waltham, Mass.). The recovered solutions in the receiver plate were then transferred individually and sequentially to the loading vials of an autosampler at 4° C. in an Agilent Technologies 1200 series HPLC system (Santa Clara, Calif.). A 100 μL aliquot from each vial was loaded onto a BioSep SEC-3000 column (Phenomenex, Torrance, Calif.) and the column effluent was monitored by a fluorescent detector at excitation and emission wavelengths of 494 nm and 519 nm, respectively. The chromatography was run at the flow-rate of 1 mL/min for a total of 20 min with 1×PBS (pH 7.3) as the mobile phase. ChemStation Software (Agilent Technologies, Santa Clara, Calif.) was used to set up and collect data from the runs automatically and continuously. The time needed to process all the calibration standards, controls, and 35 patient serum samples was ˜22 hours for a single HPLC system.

### 2.5.2. IFX Homogeneous Mobility Shift Assay (IFX-HMSA)

The procedure for the IFX-HMSA was similar to the ATI-HMSA, except that the acid dissociation step was omitted in the preparation of the patient serum samples. IFX spiked in pooled NHS were used as calibration standards. The assays were performed by incubating the TNF-488/IC with serum samples or calibration standards to reach equilibrium. As in the ATI-HMSA method, the reaction mixtures were then filtered and analyzed by the SE-HPLC system.

### 2.6. Data Analysis

Data analysis was performed with the use of a proprietary automated program run on R software (R Development Core Team, Vienna, Austria). Briefly, the R program opened the ChemStation files collected in the entire run's analyses and exported the raw spectra for an experiment of the user's choosing. The program then normalized the spectra, determined the area under each peak, and calculated the proportion of total peak areas shifted to the bound ATI/IFX-488 complexes over the total bound and free IFX-488 peak areas in the ATI-HMSA and in a similar manner for the IFX-HMSA. With these calculated data, a standard curve was generated by fitting a five-parameter logistic curve to the eight calibration samples using a non-linear least squares algorithm. The residual sum of squares (RSS) was determined to judge the quality of the fit. Using this curve function, the five optimized parameters, and each sample's proportion of shifted area, concentrations for the unknown samples and the control samples (high, mid and low) were determined by interpolation. To obtain the actual ATI and IFX concentration in the serum, the interpolated results from the standard curve were multiplied by the dilution factor.

### 2.7. ATI-HMSA and IFX-HMSA Assay Performance Validation

### 2.7.1. Characterization of the Standard Curves

Performance characteristics of the ATI-HMSA calibration standards in the concentration range of 0.006-0.720 μg/mL and the three QC samples (high, mid, and low) were monitored over 26 separate experiments, while the performance characteristics of the IFX-HMSA calibration standards in the concentration rage of 0.03-3.75 μg/mL and the three QC samples were monitored over 38 separate experiments. Standard curve performance was evaluated by both the coefficient of variation (CV) for each data point as well as the recovery percentage of the high, mid, and low QC controls. Acceptance criteria were defined as CV <20% for each QC sample.

### 2.7.2. Assay Limits Determination

The limit of blank (LOB) was determined by measuring replicates of the standard curve blanks across multiple days. The LOB was calculated using the equation: LOB=Mean+1.645×SD (Armbruster et al., Limit of blank, limit of detection and limit of quantitation. Clin Biochem Rev 29 Suppl 1, S49-S52 (2008)). The limit of detection (LOD) was determined by utilizing the measured LOB and replicates of ATI or IFX positive controls that contained a concentration of ATI or IFX that approached the LOB. The LOD was calculated using the equation: LOD=LOB+1.645×SD _{(low concentration sample)} (Armbruster et al., Limit of blank, limit of detection and limit of quantitation. Clin Biochem Rev 29 Suppl 1, S49-S52 (2008)). The lower and upper limits of quantitation (LLOQ and ULOQ, respectively) were the lowest and highest amounts of an analyte in a sample that could be quantitatively determined with suitable precision and accuracy. LLOQ and ULOQ were determined by analyzing interpolated concentrations of replicates of low concentration or high concentration serum samples containing spiked-in IFX or ATI. The LLOQ and ULOQ were each defined as the concentration that resulted in a CV<20% and standard error <25%.

### 2.7.3. Assay Precision

Nine replicates of ATI- or IFX-positive controls (high, mid, and low) were run during the same assay to measure intra-assay precision and accuracy. The minimum acceptable CV range was <20% and accuracy (% error) was <25%. Inter-assay precision was determined by running the assay standard and controls by the same analyst on different days and different instruments, followed by three analysts performing the same assay on different days with the same instrument. The minimum acceptable criteria were <20% for CV and <25% for accuracy.

### 2.7.4. Linearity of Dilution

Linearity of the ATI-HMSA and the IFX-HMSA was determined by performing a two-fold serial dilution of an ATI- or an IFX-positive sample to graphically determine the relationship between the observed and the expected concentrations. Both the R² value and the slope of each linear regression curve were calculated to evaluate the linearity of the assays.

### 2.8. Cut Point Determination

Serum samples from drug-naïve healthy donors (n=100; Golden West Biologics. Temecula, Calif.) were analyzed to determine the screen cut point for the ATI-HMSA and IFX-HMSA. We set the cut point to have an upper negative limit of approximately 97.5%. It was calculated by using the mean value of individual samples interpolated from the standard curve plus 2.0 times the standard deviation (SD), where 2.0 was the 97.5th percentile of the normal distribution. Receiver-operating characteristic analysis was also used to estimate the clinical specificity and sensitivity for the ATI-HMSA.

### 3. Results

### 3.1. HMSA Principles

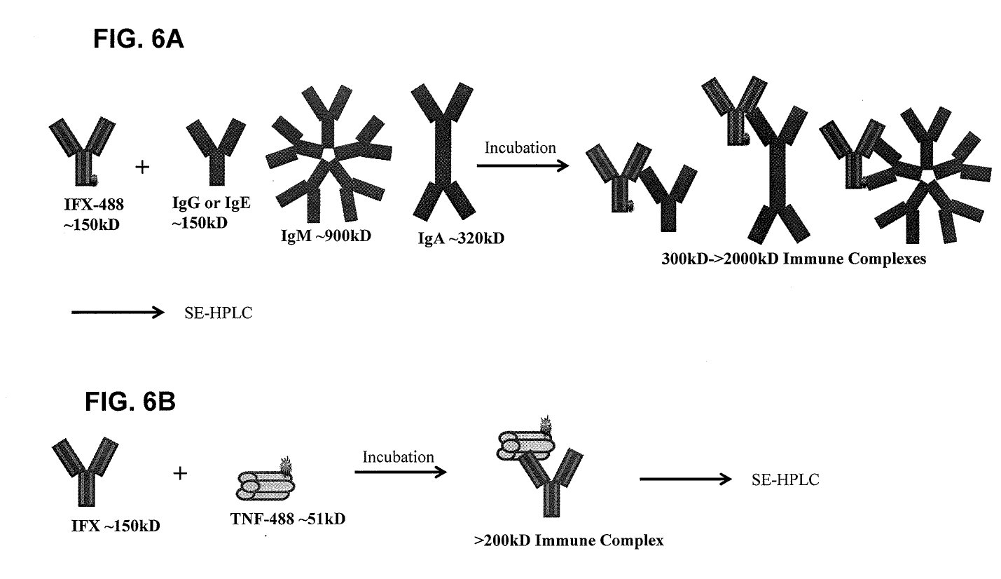

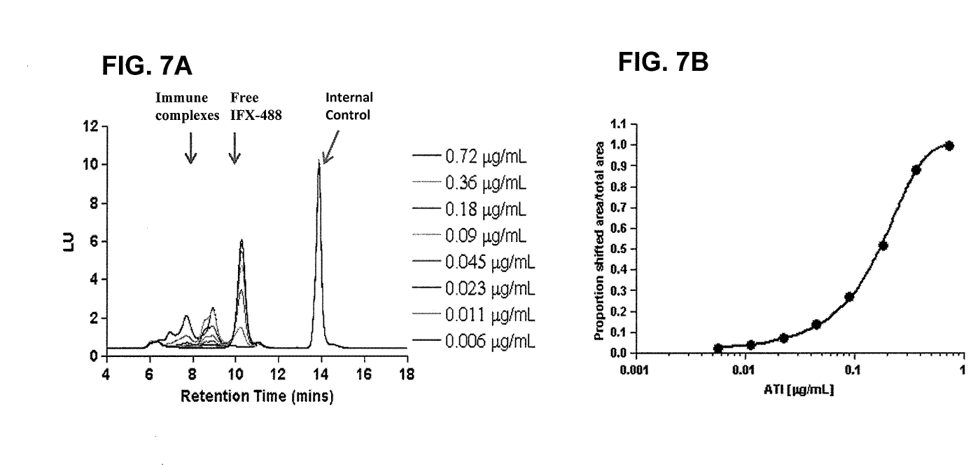

The principles of the ATI-HMSA and the IFX-HMSA are illustrated in FIGS. 6A and 6B, respectively. The ATI-HMSA in FIG. 6A involved incubating an ATI-containing serum sample with IFX-488/IC at RT for one hour to form IFX-488/ATI immune complexes. At the end of the incubation, the immune complexes and the remaining free IFX-488 were separated by SE-HPLC and the peak areas of the bound IFX-488 and the free IFX-488 were quantified by fluorescence detection. A pooled ATI-positive serum was used as the calibration standard. When serial dilutions of the ATI calibration standard were incubated with IFX-488, dose-dependent immune complexes were formed with concomitant reduction of the free IFX-488, all of which could be resolved by SE-HPLC analysis, as shown in FIG. 7A. FIG. 7B shows the standard curve generated by plotting the data from FIG. 7A. The lowest concentration of ATI in the standard curve was 0.006 μg/mL.

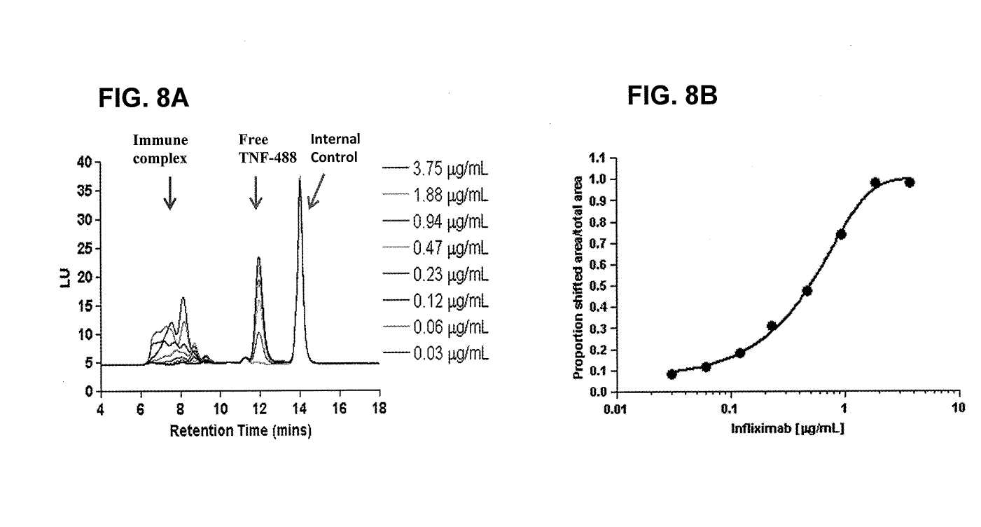

FIG. 6B illustrates the principle of the IFX-HMSA, which is similar to that of the ATI-HMSA. Incubation of the fluorescently labeled TNF-α (TNF-488) with the anti-TNF antibody IFX resulted in the formation of higher molecular weight immune complexes (TNF-488/IFX). The immune complexes and the remaining free TNF-488 were separated and quantified by SEC-HPLC. Purified IFX spiked in NHS at a concentration of 93.75 μg/mL was used as the IFX calibration standard. Using similar methodology to the ATI-HMSA, the immune complexes formed by combining the IFX calibration standards with TNF-488 were separated from the remaining free TNF-488 (FIG. 8A) and a standard curve was generated with the results (FIG. 8B).

### 3.2. Analytical Validation of ATI- and IFX-HMSA

### 3.2.1. Validation of the Standard Curve and Assay Limits

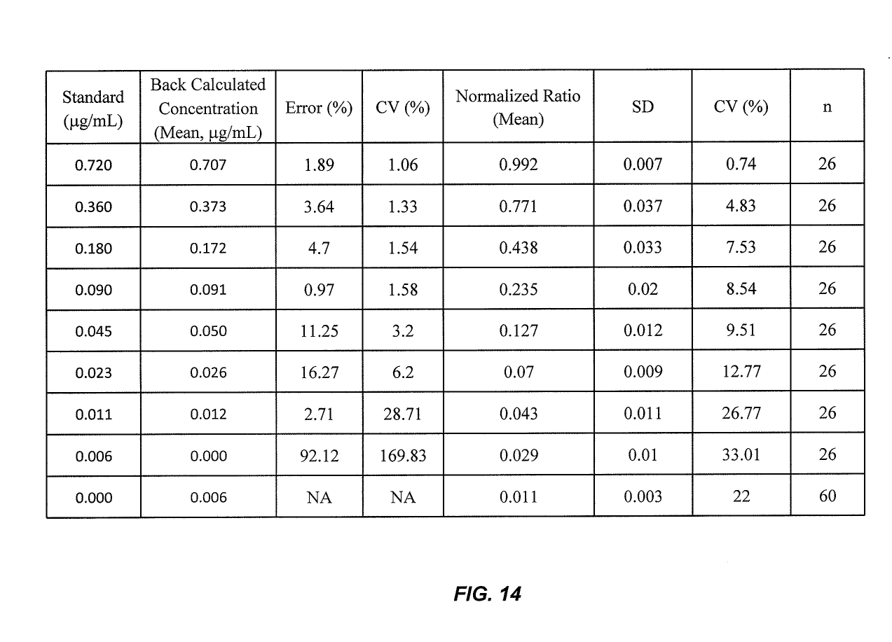

To validate the standard curve, the performance characteristics of the ATI calibration standards within the concentration range of 0.006-0.720 μg/mL were monitored over 26 experiments by multiple analysts using different instruments over different days (FIG. 14). The mean RSS for the five-parameter fitted curve was <0.001 (n=26) which was significantly better than our acceptability criterion of RSS=0.01. The error for the back-calculated values of the standards was within 30%, except for the lowest concentration (0.006 μg/mL). The CV was <10% for concentrations above 0.011 μg/mL and the dynamic range of the assay was two orders of magnitude. To establish the LOB, blank samples were tested (negative control, 0 μg/mL) along with the standard curve. The mean proportion value of the shifted area (immune complexes) over the total area determined from the blanks was 0.011±0.003 (n=60). The LOB was thus calculated to be 0.015 (mean+1.645×SD) and the extrapolated ATI concentration from the standard curve was 0.006 μg/mL. To determine the LOD, the extrapolated value of the lowest standard concentration (0.006 μg/mL) was obtained as 0.014±0.003 μg/mL (n=26). The LOD was calculated from the LOB and the SD from the lowest concentration in the standard curve with <20% error: LOD=LOB+1.645×SD _{(low concentration sample)} which was 0.012 μg/mL. The LLOQ for the ATI-HMSA assay was 0.011 μg/mL, which was determined by the interpolated concentrations of replicates of the low ATI concentration with CV<20%. The ULOQ for the ATI-HMSA assay was 0.54 μg/mL, which was similarly determined by the interpolated concentrations of replicates of the high ATI concentration with CV<20%. The effective serum concentrations corresponding to the LLOQ and the ULOQ for the ATI-HMSA were determined by multiplying the concentration with the dilution factor (50), which corresponded to 0.56 μg/mL and 27 μg/mL, respectively.

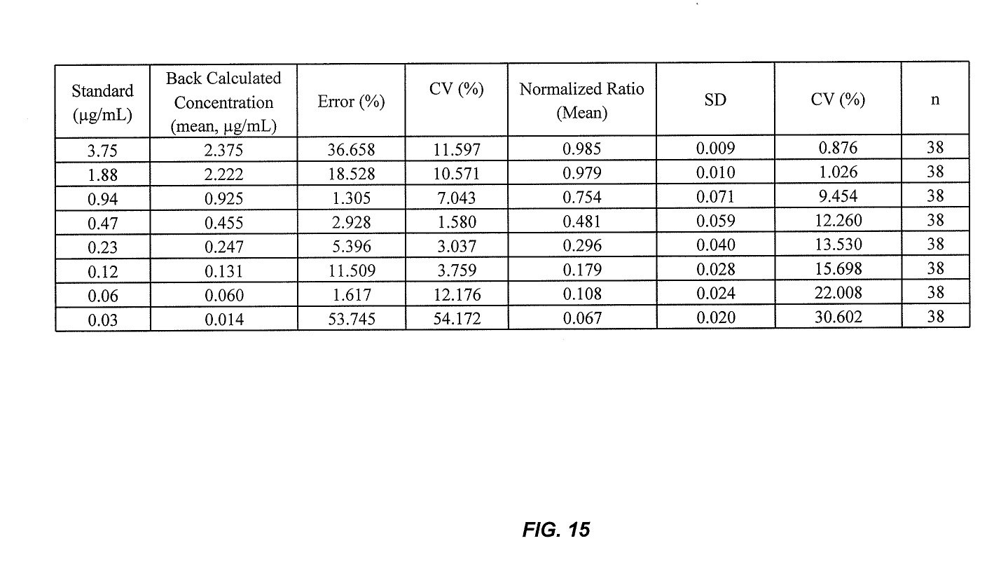

The performance characteristics of the IFX-HMSA standard curve in the concentration range of 0.03-3.75 μg/mL were similarly assessed over 38 experiments by multiple analysts using different instruments on different days (FIG. 15). The same methods were used to determine the LOB, LOD, LLOQ, and ULOQ as described for the ATI-HMSA. The LOB, LOD, LLOQ, and ULOQ for the IFX-HMSA were 0.0027, 0.0074, 0.039, and 1.36 μg/mL, respectively. The effective IFX serum concentration for the LLOQ and ULOQ were 0.98 and 34 μg/mL (dilution factor=25).

### 3.2.2. Assay Precision and Accuracy

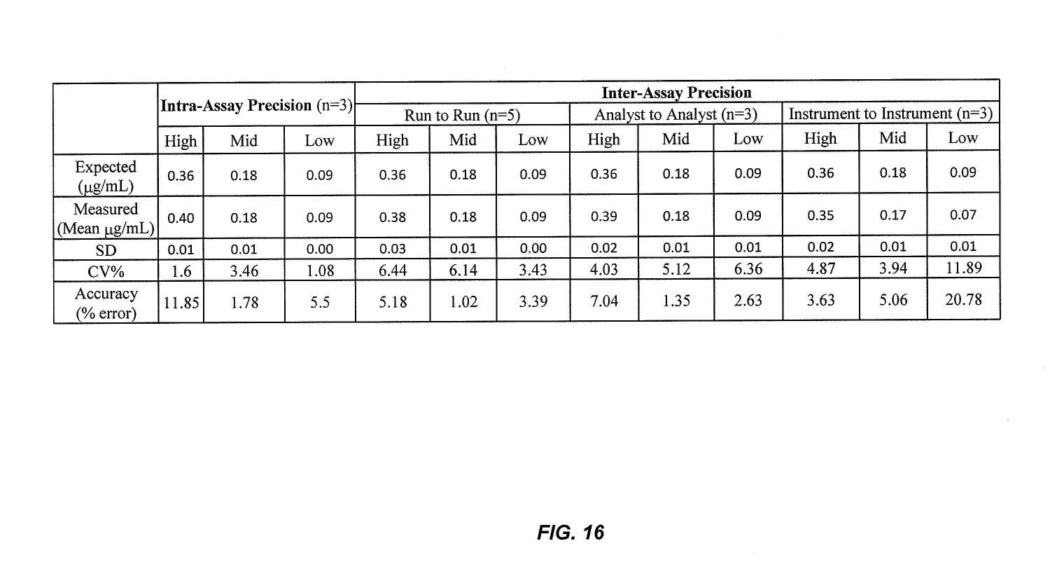

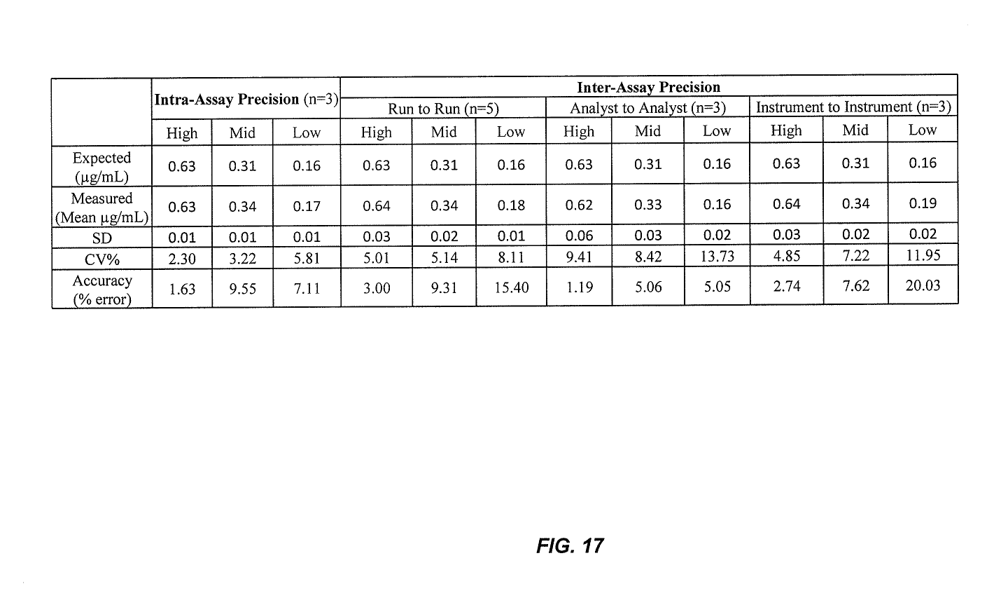

To assess the precision and accuracy of the ATI-HMSA and the IFX-HMSA, two methods were used. First, we used the high, mid, and low QC samples in both assays to determine their recovery rate. As shown in FIG. 16, the ATI-HMSA intra-assay precision had a CV<4% and the accuracy rate was <12% error. The intra-assay precision and accuracy for the IFX-HMSA were <6% and <10% error, respectively (FIG. 17). Second, we tested the high, mid, and low control samples over different runs and instruments and by multiple analysts. For both assays, the inter-assay precision had a CV<15% and the accuracy were <21% error, both of which were within the acceptable limits (FIGS. 16 and 17).

### 3.2.3. Linearity of Dilution

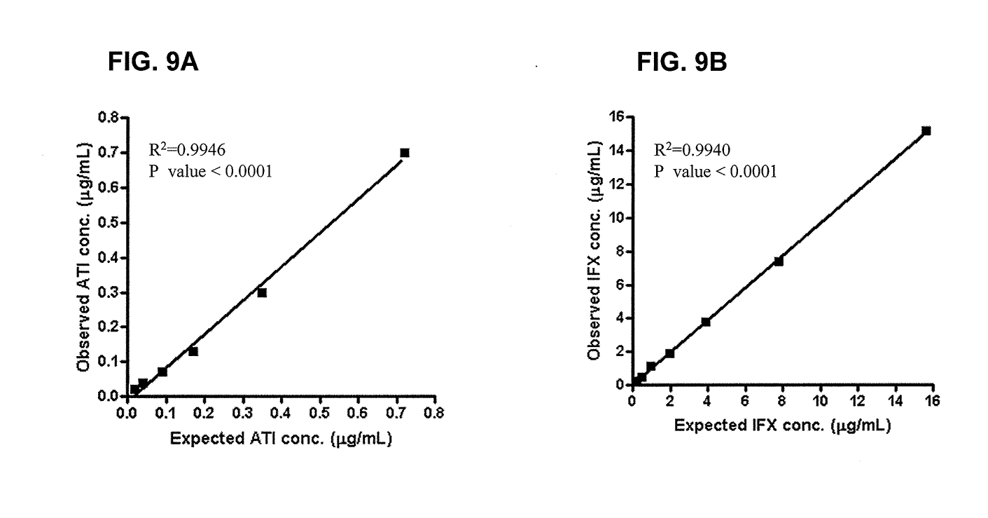

To ensure accurate quantitative assessment, the positive samples of the assay must dilute linearly and in parallel with the standard curve. To determine this linearity of dilution, human serum samples containing a high titer of ATI or a high concentration of IFX were used. The samples were diluted serially 2-fold and tested using the ATI-HMSA and the IFX-HMSA, respectively. The observed values of ATI or IFX were plotted with the expected levels of ATI or IFX in the serum. As shown in FIGS. 9A and 9B, both the R² values and the slopes of each linear regression curve for both assays show linearity.

### 3.2.4. Effects of Potential Interfering Substances

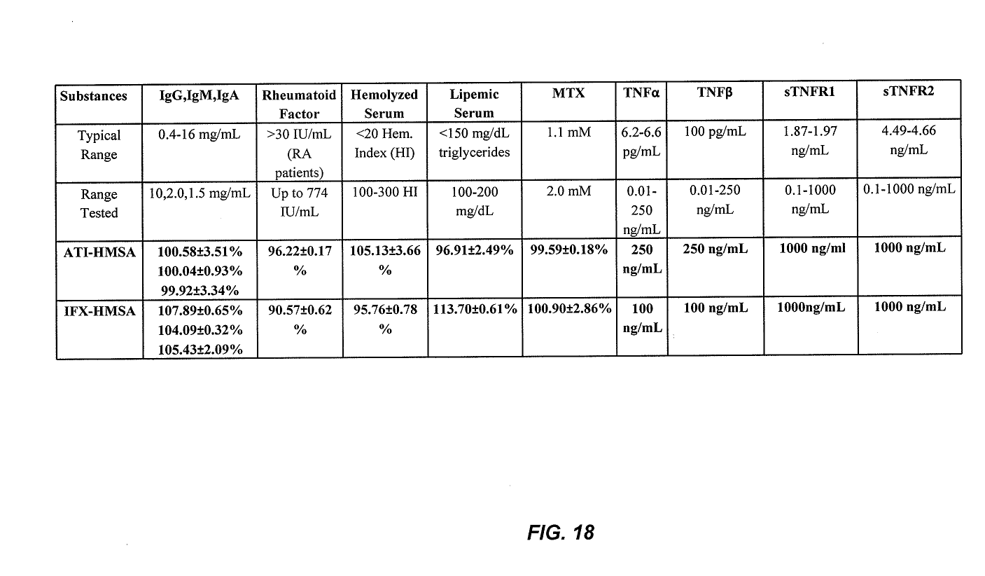

We studied the effects of potential substance interference in both assays by spiking in common endogenous components of human serum and the drug methotrexate (MTX) into the three QC samples (high, mid, and low) to determine their percent recovery. As shown in FIG. 18, no significant interference was observed in the physiological levels of immunoglobulin, rheumatoid factor, hemolyzed serum, lipemic serum, and MTX in both assays as assessed by the recovery of the ATI and IFX QC samples in the presence of the potential interfering substances. For TNFα, TNFβ, sTNFR1, and sTNFR2 some interference was observed when the concentration of these substances was spiked in at over 1000x physiological levels, as shown in FIG. 18.

### 3.3. IFX Drug Tolerance of the ATI-HMSA

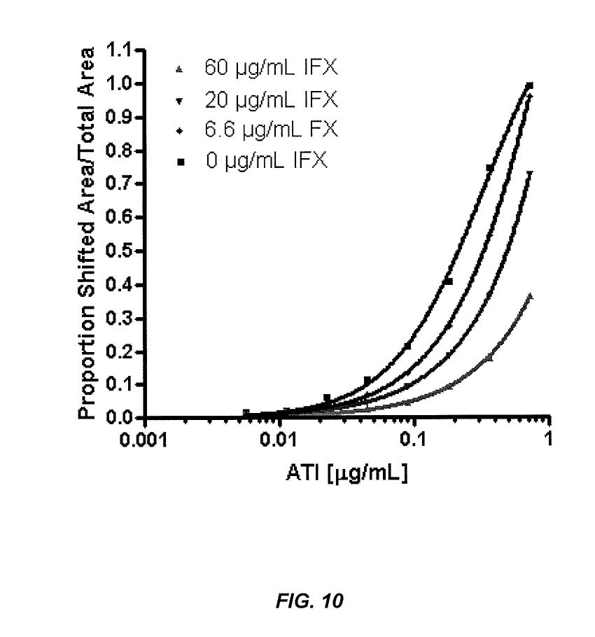

Substantial concentrations of IFX may be present in the serum from patients, even if the blood is drawn at the trough time point. As discussed previously, the presence of IFX in the patient serum significantly affected the quantitative measurement of ATI using the bridging ELISA assay. To address this issue with the HMSA-based assays, we evaluated the potential impact of IFX level in patient serum on ATI-HMSA results by adding increased amounts of IFX (6.6, 20, and 60 μg/mL) to each of the eight ATI calibration standards to assess the effects on the standard curve. As seen in FIG. 10, the ATI-HMSA could detect ATI levels as low as 0.036 μg/mL in the serum sample containing up to 60 μg/mL of IFX, which is much higher than the maximum therapeutic level reached after infusion of the patient with IFX.

### 3.4. Cut Point Determinations for the ATI-HMSA and IFX-HMSA

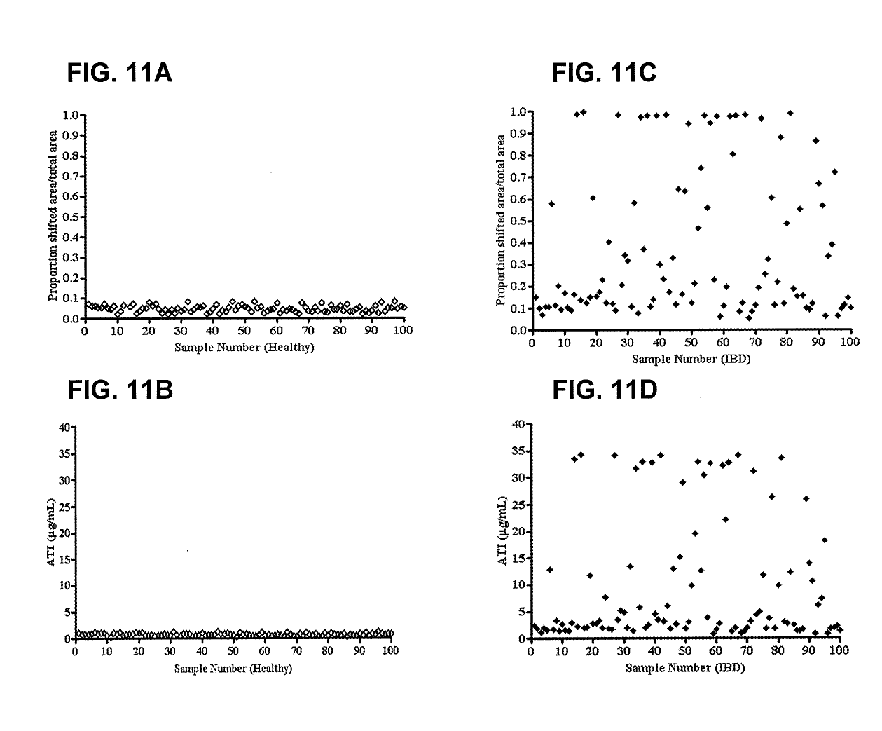

To establish the cut point for the ATI-HMSA and the IFX-HMSA, we screened 100 serum samples collected from IFX drug-naïve healthy subjects for the measurement of ATI and IFX levels. No shifting of the IFX-488 to the bound complex areas was found in most of the samples of the ATI-HMSA (FIG. 11A). The proportion of shifted area over the total area was near the LOB and the mean value of the extrapolated ATI from standard curve (multiplied by the dilution factor) was 0.73±0.23 μg/mL as shown in FIG. 11B. The cut point for ATI was determined by taking the mean value+2×SD, which yielded 1.19 μg/mL. Three samples contained ATI levels slightly higher than the cut point, which resulted in a clinical specificity of 97%. The same 100 serum samples were also used to establish the cut point for the IFX-HMSA (data not shown). The calculated cut point for IFX-HMSA was 0.98 μg/mL, yielding a clinical specificity of 95%.

### 3.5. Clinical Validation of the ATI-HMSA

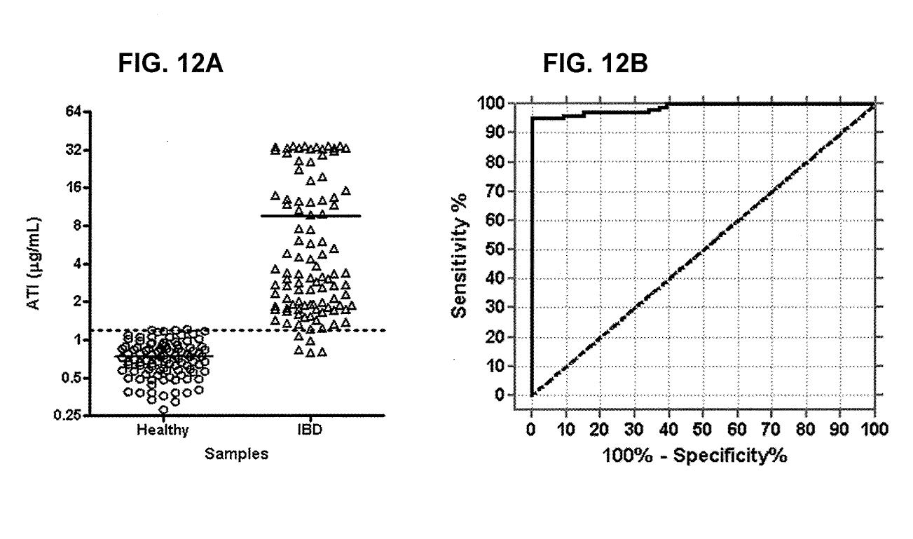

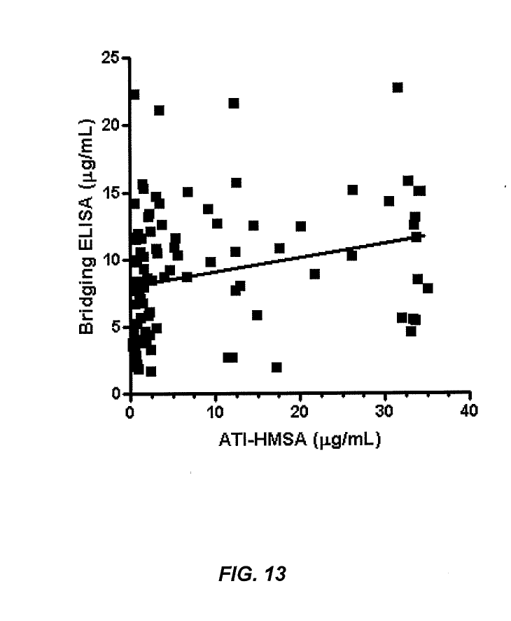

Currently, one of the clinically validated methods for measuring ATI is by using bridging ELISA methodology (Baert et al., Influence of immunogenicity on the long-term efficacy of infliximab in Crohn's disease, N. Engl. J Med 348, 601 (2003)), which over the last decade has been used to measure ATI in serum samples from IBD patients treated with IFX. To evaluate the performance of the HMSA to detect ATI in the presence of IFX compared to that of the bridging ELISA assay, we performed ATI-HMSA on 100 serum samples obtained from IBD patients that were previously tested to be positive for ATI by the bridging ELISA method. The proportion of shifted area over the total area and the interpolated ATI from the standard curve (multiplied by the dilution factor of 50) are shown in FIGS. 11C and D, respectively. The mean values of ATI in the patient serum samples were significantly higher than those in the drug-naïve healthy controls (mean±SD=9.57±11.43 vs. 0.73±0.29 μg/mL, p<0.0001) as shown in FIG. 12A. Receiver operating characteristic curve analysis of these samples (FIG. 12B) showed that the area under the curve was 0.986±0.007 (95% CI: 0.973-0.999, p<0.0001), the sensitivity was 95% (95% CI: 88.72%-98.36%), and the odds ratio was 47.50 when a 1.19 μg/mL cut point was used. Good correlation between the ATI values obtained from the ATI-HMSA and the bridging ELISA was also observed, with p<0.0001 and a Spearman r-value of 0.39 (95% CI: 0.2-0.55) as shown in FIG. 13. Upon re-testing the three samples from the healthy controls with the ATI concentration above the cut point (1.196, 1.201, and 1.219 μg/mL) using ATI-HMSA, the resulting ATI concentrations were all below the cut point. Thus we defined these results as false-positive. However, among the 100 ATI-positive IBD patient serum samples previously determined by the bridging ELISA, five of the samples were found to be ATI-negative (i.e., containing ATI concentrations below the cut point of 1.19 μg/mL). Repeatedly re-testing these samples showed no shift on the SE-HPLC chromatogram, thus we defined the five samples as true negative. The increased rate of false-positive ATI measurements with the bridging ELISA method may be attributed to an elevated level of nonspecific binding. By significantly reducing this limitation, the HMSA method can provide higher specificity for detecting ATI than the bridging ELISA method.

### 4. Discussion

Since the initial approval of the antibody drug IFX by the United States Food and Drug Administration for the treatment of Crohn's disease (CD) in 1998, the broad use of anti-TNF therapy in IBD has dramatically improved therapeutic outcome over the past decade (Targan et al., A short-term study of chimeric monoclonal antibody cA2 to tumor necrosis factor alpha for Crohn's disease, Crohn's Disease cA2 Study Group, N. Engl. J. Med 337, 1029 (1997); Colombel et al., Infliximab, azathioprine, or combination therapy for Crohn's disease, N. Engl. J. Med 362, 1383 (2010); Present et al., Infliximab for the treatment of fistulas in patients with Crohn's disease, N. Engl. J. Med 340, 1398 (1999); Rutgeerts et al., Efficacy and safety of retreatment with anti-tumor necrosis factor antibody (infliximab) to maintain remission in Crohn's disease, Gastroenterology 117, 761. (1999); Hanauer et al., Maintenance infliximab for Crohn's disease: the ACCENT I randomised trial, Lancet 359, 1541 (2002)). Nevertheless, there is a significant number of patients that either fail to respond (primary non-responders) or lose response (secondary non-responders) to anti-TNF treatments. There are many factors that may contribute to the loss of response to IFX in IBD patients, such as the development of a complication to the disease or uncontrolled disease activity (Miheller et al., Anti-TNF trough levels and detection of antibodies to anti-TNF in inflammatory bowel disease: are they ready for everyday clinical use? Expert Opin. Biol Ther. 12, 179 (2012)), in addition to the formation of ATI. ATI formation negatively affects drug efficacy by increasing the clearance of IFX and/or neutralizing its activity, therefore reducing the amount of active IFX in circulation (Baert et al., Influence of immunogenicity on the long-term efficacy of infliximab in Crohn's disease, N. Engl. J. Med 348, 601 (2003); Hanauer et al., Incidence and importance of antibody responses to infliximab after maintenance or episodic treatment in Crohn's disease, Clin Gastroenterol. Hepatol. 2, 542 (2004); Farrell et al., Intravenous hydrocortisone premedication reduces antibodies to infliximab in Crohn's disease: a randomized controlled trial, Gastroenterology 124, 917 (2003); Miele et al., Human antichimeric antibody in children and young adults with inflammatory bowel disease receiving infliximab, J Pediatr Gastroenterol. Nutr. 38, 502 (2004)). In contrast, achieving an adequate serum IFX level is not only associated with improved treatment response but also appears to have a lower rate of ATI formation (Maser et al., Association of trough serum infliximab to clinical outcome after scheduled maintenance treatment for Crohn's disease, Clin Gastroenterol Hepatol 4, 1248 (2006); Farrell et al., Intravenous hydrocortisone premedication reduces antibodies to infliximab in Crohn's disease: a randomized controlled trial, Gastroenterology 124, 917 (2003)). Thus, there is an interdependent relationship between IFX levels and ATI, which underscores the importance of measuring and monitoring both IFX and ATI levels accurately. An evolving concept in the management of IBD patients with biologic therapy involves dose optimization using an individualized dosing regimen versus a standard “one-dose-fits-all” regimen to attain a personalized target therapeutic drug level (Ordas et al., Anti-TNF Monoclonal Antibodies in Inflammatory Bowel Disease: Pharmacokinetics-Based Dosing Paradigms, Clinical Pharmacology and Therapeutics 91, 635 (2012)). This concept was demonstrated in a clinical study that correlated patient trough serum IFX concentration with response and remission (Maser et al., Association of trough serum infliximab to clinical outcome after scheduled maintenance treatment for Crohn's disease, Clin Gastroenterol Hepatol 4, 1248 (2006)). Recently, these findings were supported by a study of 115 UC patients where it was found that a detectable trough serum IFX level predicted clinical remission, endoscopic improvement, and a lower risk for colectomy, whereas, an undetectable trough serum IFX level was associated with less favorable outcomes (Seow et al., Trough serum infliximab: a predictive factor of clinical outcome for infliximab treatment in acute ulcerative colitis, Gut 59, 49 (2010)). This proposed treatment strategy is in contrast to the most commonly used strategies of empirically increasing the dose, shortening the infusion frequency, or switching to another anti-TNF agent such as adalimumab or certolizumab pegol. A growing body of evidence suggests that serial monitoring of serum drug and ADA levels are important in the management and optimization of these therapies and thus may increase the overall response, the duration of response, and minimize adverse effects (Ordas et al., Anti-TNF Monoclonal Antibodies in Inflammatory Bowel Disease: Pharmacokinetics-Based Dosing Paradigms, Clinical Pharmacology and Therapeutics 91, 635 (2012)).

Many clinicians have advocated the concurrent measurement of serum ATI and IFX levels in patients treated with IFX or other anti-TNF drugs and, indeed, monitoring of various anti-TNF drugs and their respective antibodies in IBD and RA patients has been studied in several clinical trials using a variety of methods (Miheller et al., Anti-TNF trough levels and detection of antibodies to anti-TNF in inflammatory bowel disease: are they ready for everyday clinical use? Expert Opin. Biol Ther. 12, 179 (2012); Guerra et al., Utility of measuring serum concentrations of anti-TNF agents and anti-drug antibodies in inflammatory bowel disease, Curr. Drug Metab 12, 594 (2011)). Different assay techniques were used to measure the ATI and IFX concentrations in the different trials, which may contribute to the inconsistent results obtained between studies. Many ELISA methods with different formats are available for commercial use, but the reliability of these methods may be questionable because there is no standard available for comparison. The most common method for measuring serum ATI is the bridging ELISA as described by Baert et al. (Baert et al., Influence of immunogenicity on the long-term efficacy of infliximab in Crohn's disease, N. Engl. J Med 348, 601 (2003)). However, other ELISA methods have also been described to detect IFX and ATI in serum samples from IBD and RA patients (Bendtzen et al., Individualized monitoring of drug bioavailability and immunogenicity in rheumatoid arthritis patients treated with the tumor necrosis factor alpha inhibitor infliximab, Arthritis Rheum. 54, 3782 (2006); Bendtzen et al., Individual medicine in inflammatory bowel disease: monitoring bioavailability, pharmacokinetics and immunogenicity of anti-tumour necrosis factor-alpha antibodies, Scand. J. Gastroenterol. 44, 774 (2009); Ben-Horin et al., The decline of anti-drug antibody titres after discontinuation of anti-TNFs: implications for predicting re-induction outcome in IBD. Aliment, Pharmacol Ther. 35, 714 (2012); Imaeda et al., Development of a new immunoassay for the accurate determination of anti-infliximab antibodies in inflammatory bowel disease, J Gastroenterol 47, 136 (2012)). Some of these assays appear to be capable of detecting ATI in the presence of low concentrations of IFX, but the ATI-positive rates determined by these methods varied significantly (Kopylov et al., Clinical utility of antihuman lambda chain-based enzyme-linked immunosorbent assay (ELISA) versus double antigen ELISA for the detection of anti-infliximab antibodies, Inflamm Bowel Dis Published on line 29 OCT 2011 (2011); Imaeda et al., Development of a new immunoassay for the accurate determination of anti-infliximab antibodies in inflammatory bowel disease, J Gastroenterol 47, 136 (2012)). RIA has also been developed to measure serum ATI and IFX concentrations, and their clinical utility was compared to solid-phase ELISA methods (Wolbink et al., Development of antiinfliximab antibodies and relationship to clinical response in patients with rheumatoid arthritis, Arthritis Rheum. 54, 711 (2006); Bendtzen et al., Individualized monitoring of drug bioavailability and immunogenicity in rheumatoid arthritis patients treated with the tumor necrosis factor alpha inhibitor infliximab, Arthritis Rheum. 54, 3782 (2006); Svenson et al., Monitoring patients treated with anti-TNF-alpha biopharmaceuticals: assessing serum infliximab and anti-infliximab antibodies, Rheumatology, (Oxford) 46, 1828 (2007)). In general, RIA has some advantages over ELISA with fewer artifacts. However, RIA methodology is more complex compared to ELISA methodology and the use of radioactive materials is a major issue in many clinical labs. Nevertheless, despite the different ATI and IFX results obtained using the various methods, the clinical outcomes from most of the studies were similar, namely: 1) Detectable levels of ATI or high-titer ATI were correlated with low concentrations or undetectable trough levels of IFX, respectively, and 2) Patients who were ATI-positive and possessed low trough levels of IFX had a higher rate of loss of response to IFX treatment.

By taking advantage of homogenous fluid-phase methodology and avoiding the multiple washing steps of the ELISA format, we have developed an HMSA method with the ability to quantitatively measure IFX drug and ATI levels in IBD patient serum samples. This method was based on the incubation of IBD patient serum samples with fluorescent-labeled IFX to detect ATI levels or with fluorescent-labeled TNFα to detect IFX levels. The immune complexes formed in the incubation mixture were separated from the free label by SE-HPLC and the amount of ATI or IFX in the samples was calculated from the resolved peak areas. A far more cumbersome method had been applied to measure the formation, distribution, and elimination of IFX and anti-IFX immune complexes in cynomolgus monkeys (Rojas et al., Formation, distribution, and elimination of infliximab and anti-infliximab immune complexes in cynomolgus monkeys, J Pharmacol Exp. Ther. 313, 578 (2005). The HMSA method advantageously overcomes many potential artifacts encountered in the solid-phase ELISA method because the antibody and antigen binding reactions take place in a homogeneous liquid-phase condition. Also, the solid-phase ELISA method may only be able to detect high affinity antibodies because it involves many steps of washing and incubation that may potentially remove the antibodies bound with low affinity. Further advantages of the HMSA method include the detection of all immunoglobulin isotypes and all subclasses of IgG, including IgG₄. Analytical validation of the ATI- and IFX-HMSA showed that the assay performance was robust and not affected by potential interfering substances present in serum. Incorporation of an acid-dissociation step during ATI-HMSA dramatically improved the drug tolerance of the assay and allowed for an accurate detection of ATI in the presence of high levels of IFX (up to 60 μg/mL) in serum. The use of fluorescent labeling and fluorescent monitoring of the SE-HPLC peaks significantly increased the analytical sensitivity for measuring ATI, which can reach a concentration of 0.011 μg/mL, compared with the suboptimal concentration of 200-500 ng/mL achieved by bridging ELISA. Re-analysis of clinical samples which had previously tested positive using a bridging ELISA method showed that 5% of them were negative using ATI-HMSA; otherwise, there was good correlation between the two assays on the ATI-positive samples. The false positive rate with the cut point of 1.19 μg/mL was 3%. However, this rate could be reduced by repeating the test if the result is within 10% of the cut point (i.e., 1.19-1.21 μg/mL). Because a variety of anti-TNF drugs have been shown to induce antibody formation in clinical studies (Bartelds et al., Development of antidrug antibodies against adalimumab and association with disease activity and treatment failure during long-term follow-up, JAMA 305, 1460 (2011); Karmiris et al., Influence of trough serum levels and immunogenicity on long-term outcome of adalimumab therapy in Crohn's disease, Gastroenterology 137, 1628 (2009); Lichtenstein et al., Continuous therapy with certolizumab pegol maintains remission of patients with Crohn's disease for up to 18 months, Clin Gastroenterol Hepatol 8, 600 (2010)), the HMSA method may be applied to measure other antibody drug levels and anti-drug antibodies in patient serum samples.

In conclusion, the liquid-phase HMSA methodology presented in this example for the measuring ATI and IFX in IBD patient serum samples overcomes many limitations encountered in the solid-phase ELISA and RIA methods. Validation of the ATI- and IFX-HMSA also showed higher sensitivity and drug tolerance compared to that achieved by the ELISA method. This liquid-phase HMSA format is a useful platform that can be broadly applied to detect anti-drug antibodies and drug levels for a variety of protein therapeutics during drug development and post-approval monitoring.

### Example 8

### Comparison of Homogeneous Mobility Shift Assay and Solid Phase ELISA for the Measurement of Drug and Anti-Drug Antibody (ADA) Levels in Serum from Patients Treated with Anti-TNF Biologics

Anti-TNF monoclonal antibodies, such as infliximab (IFX), are prescribed for the treatment of inflammatory bowel disease. However, certain patients will generate ADA that can cause loss of drug efficacy and adverse reactions. The most widely used method for monitoring both drug and ADA levels in patients is the solid phase ELISA. Solid phase assays suffer from a variety of problems, including the inability to detect ADA in the presence of significant concentrations of drug. Our clinically validated, liquid phase mobility shift assay (MSA) was used to study IFX and antibody-to-infliximab (ATI) levels in patient serum and compared to ELISA. MSA correlates with commercially available ELISA assays yet overcomes many of the associated problems and can readily detect any anti-TNF biologic.

Methods:

To perform the MSA, Alexa488 labeled TNF-α is incubated with serum containing IFX (standards, controls and unknowns).

After equilibration, free Alexa488 labeled TNF-α and complexes of Alexa488 labeled TNF-α and IFX are resolved by size exclusion HPLC and the peaks quantified by fluorescence. The proportion of complex area in the standards is plotted against IFX concentration, fit to a 5-parameter logistic model to generate a standard curve and the unknowns interpolated from it. Standard curves for IFX, adalimumab (ADL), golimumab (GLM) and etanercept (ETN) were also generated using the above procedure. Similar methodology and analysis is used to measure the level of ADA in the serum. Solid phase ELISA data was generated in-house using clinically approved, commercially available assays. All samples were tested in a blinded fashion for assay comparisons.

Results:

Optimization of the MSA has lowered the IFX limit of quantitation (LOQ) from 0.98 μg/mL to 50 ng/mL and expanded the dynamic testing range (50 ng/mL-34 μg/mL), dramatically improving performance over the commercial ELISA (1.4 μg/mL LOQ, 1.4-25 μg/mL range). Detection of IFX levels in the presence of ATI is accurate up to 100 U/mL ATI, whereas ATI disrupts accuracy of ELISA data at less than 10 U/mL. The liquid phase ATI assay can be performed across an extended dynamic range compared to the limited ELISA assay range (3.13-200 U/mL MSA vs. 1.69-30 μg/mL ELISA). Furthermore, the dilution curve of the ATI assay is linear, even in the presence of drug concentration up to 60 μg/mL. The standard curves generated for each drug show high reproducibility, dynamic range, and sensitivity (<1.0 μg/mL).

Conclusions:

Homogeneous MSA demonstrates higher sensitivity, dynamic range, and less interference than solid phase ELISA. It allows for the accurate detection of ADA in the presence of drug, which was previously not possible. The liquid phase assay can be used for the detection of IFX, ADL, ETN and GLM along with the associated ADA.

### Example 9

### Comparison of Homogeneous Mobility Shift Assay and Solid Phase ELISA for The Measurement of Drug and Anti-Drug Antibody Levels in Serum from Patients Treated with Anti-TNF Biologics Such as Adalimumab

### Premise

Anti-TNF monoclonal antibodies, such as infliximab (IFX), adalimumab (ADL), and others are prescribed for the treatment of inflammatory bowel disease. Certain patients will generate anti-drug antibodies (ADA) that can cause loss of drug efficacy and adverse reactions. The most widely used method for monitoring both drug and ADA levels in patients is the solid phase ELISA. Solid phase assays suffer from a variety of problems, including the inability to detect ADA in the presence of significant concentrations of drug. Our clinically validated, liquid phase homogeneous mobility shift assay (HMSA) was used to study IFX and antibody-to-infliximab (ATI) levels as well as ADL and antibody-to-adalimumab (ATA) in patient serum and compared to ELISA. HMSA can readily detect any anti-TNF biologic with improved sensitivity and dynamic range.

### Methods

To perform the HMSA, Alexa488 labeled TNF-α is incubated with serum containing IFX or ADL (standards, controls and unknowns). After equilibration, free Alexa488 labeled TNF-α and complexes of Alexa488 labeled TNF-α and IFX are resolved by size exclusion HPLC and the peaks quantified by fluorescence. The proportion of complex area in the standards is plotted against IFX or ADL concentration, fit to a 5-parameter logistic model to generate a standard curve and the unknowns interpolated from it. Statistical programming language R was used for all data analysis. Standard curves for IFX, ADL, golimumab (GLM) and etanercept (ETN) were also generated using the above procedure.

For ATI and ATA experiments, serum samples were first acid dissociated with 0.5M citric acid, pH 3.0 for one hour at room temperature. Following the dissociation, excess IFX/ADL-Alexa488/internal control was added and the reaction mixture was immediately neutralized with 10×PBS, pH 7.3. After neutralization, the reaction mixture was incubated for another hour at room temperature on a plate shaker to complete the reformation of the immune-complexes. The samples were then filtered and analyzed by SEC-HPLC as described above.

ATI HMSA data was converted from U/mL to μg/mL using a conversion factor based on the free IFX HPLC peak AUC observed in the HMSA (linear correlation observed when specific, 1:1 binding of IFX/ATI complexes assumed).

Serum ADL and ATA concentrations were measured in IBD patients from an IRB-approved study and in healthy controls purchased from Golden West Biologics, Inc. Solid phase ELISA data was generated using clinically approved, commercially available assays. All samples were tested in a blinded fashion for assay comparisons.

### Results

Comparison of HMSA and ELISA.

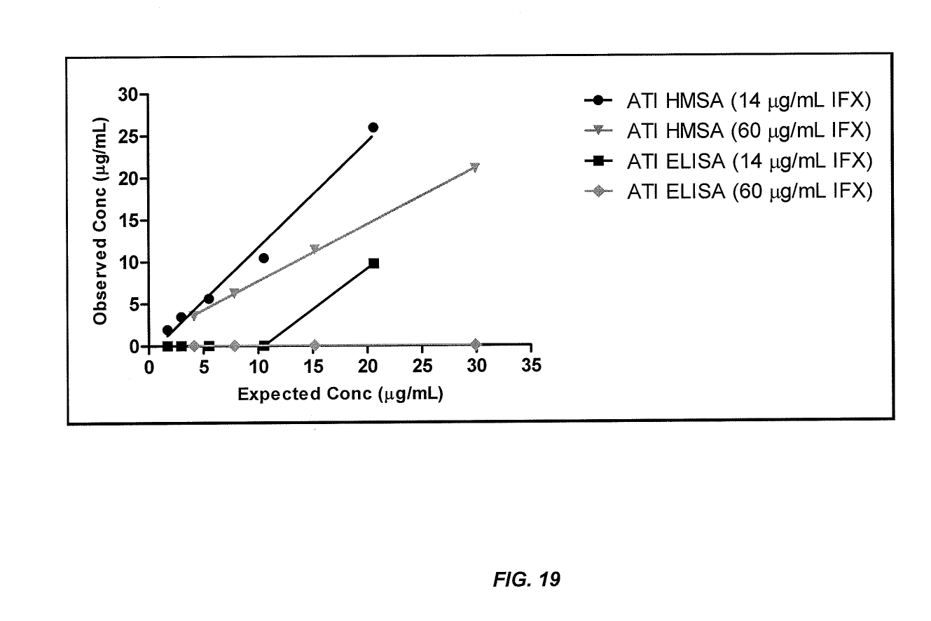

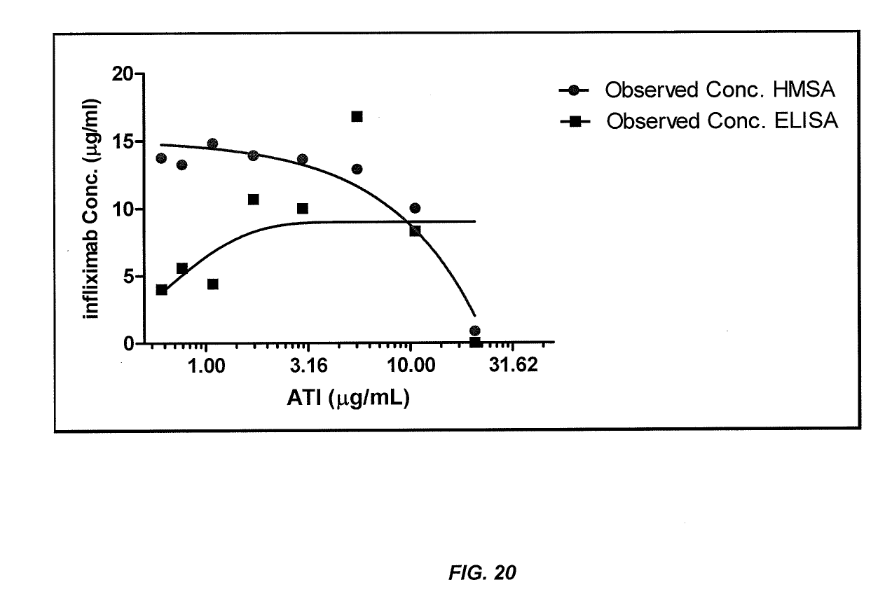

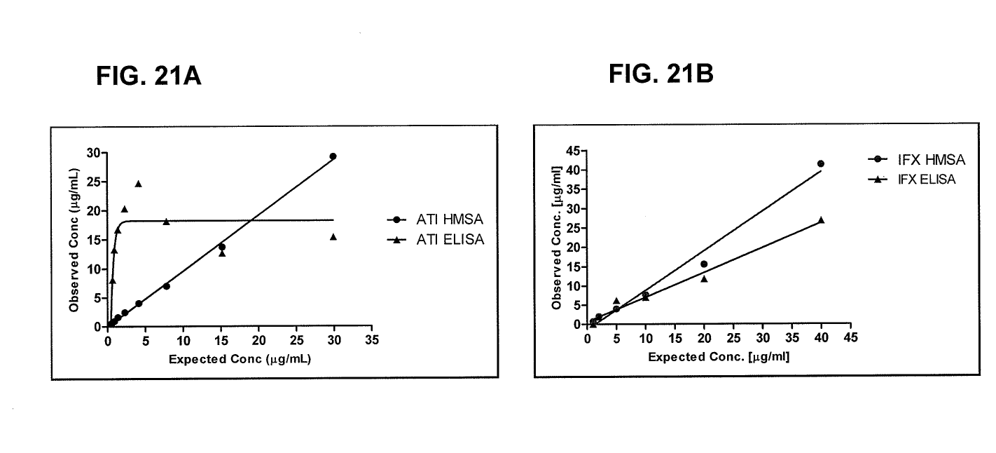

The HMSA allows measurement of samples containing both IFX and ATI that would be designated “inconclusive” by traditional ELISA methods (FIGS. 19 and 20). To compare the dynamic range of the two methodologies, serum samples containing a known concentration of IFX or ATI were serial diluted 2-fold in normal human serum and then tested by either the HMSA or ELISA (FIGS. 21A and 21B).

Development of High Sensitivity IFX Assay.

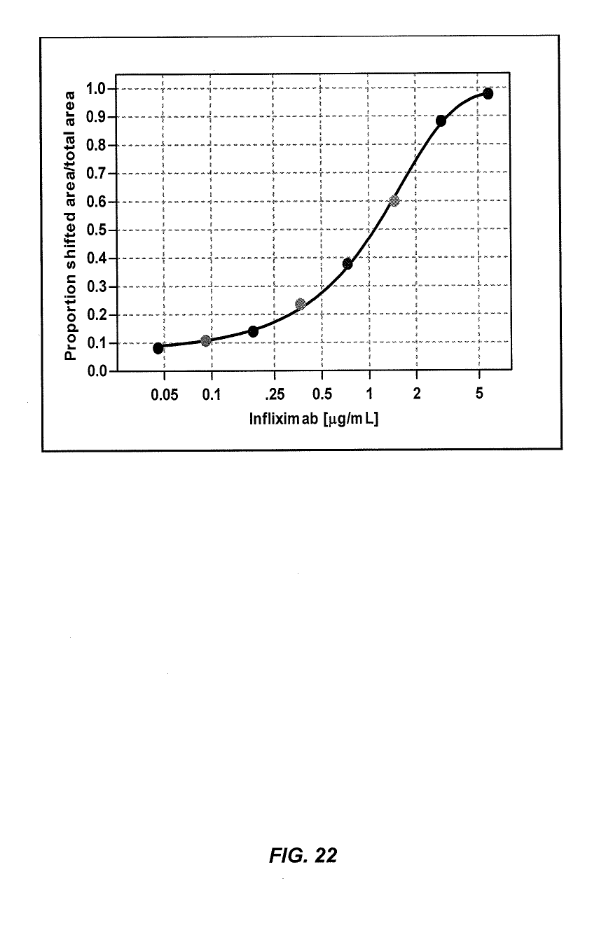

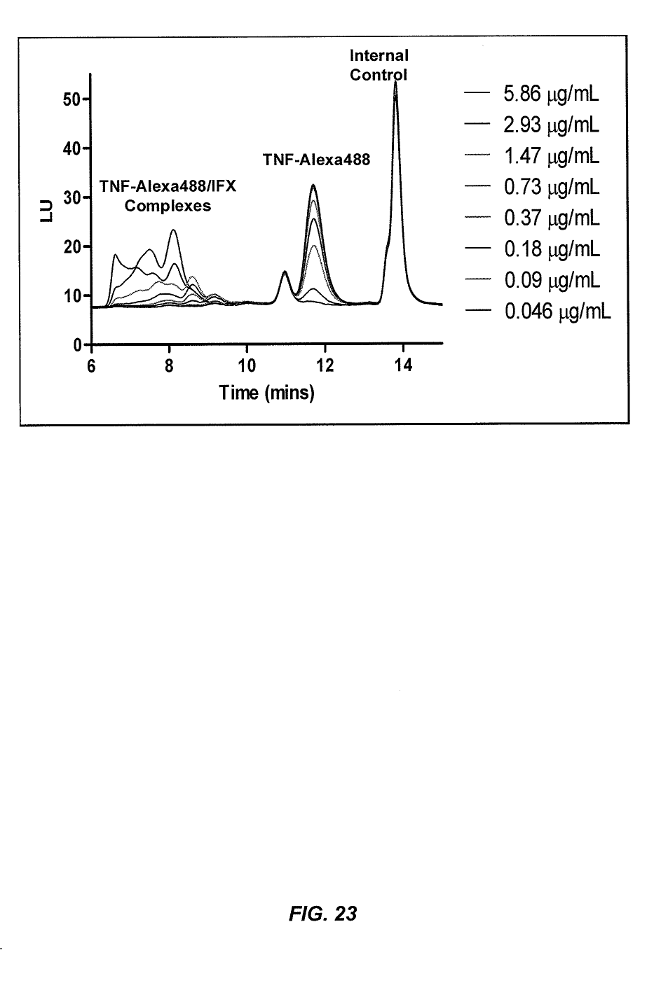

To increase the sensitivity, the fluorescence detector parameters were optimized based on the results of amplification plots and isoabsorbance plots from healthy and IBD patient serum samples. In some embodiments, the infliximab mobility shift assay detects as little as 50 ng/mL of infliximab in serum with high reproducibility. See, FIGS. 22 and 23; see also, Table 2.

TABLE 2
High sensitivity IFX assay.

| Sensitivity Measurement (n = 20) | Value |
|---|---|
| LOD (ng/mL) | 13.00 |
| LLOQ (ng/mL) | 51.02 |
| LLOQ CV (%) | 21.07 |
| LLOQ Error (%) | 11.40 |

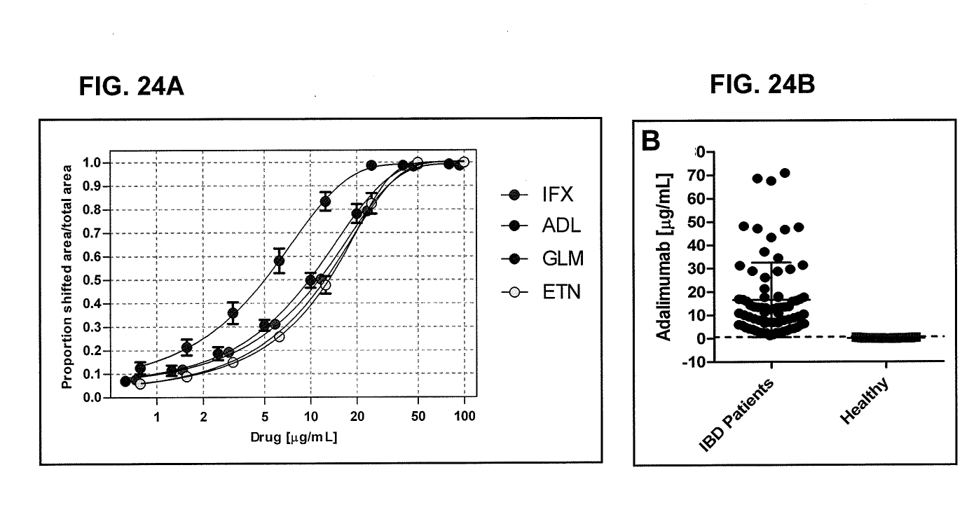

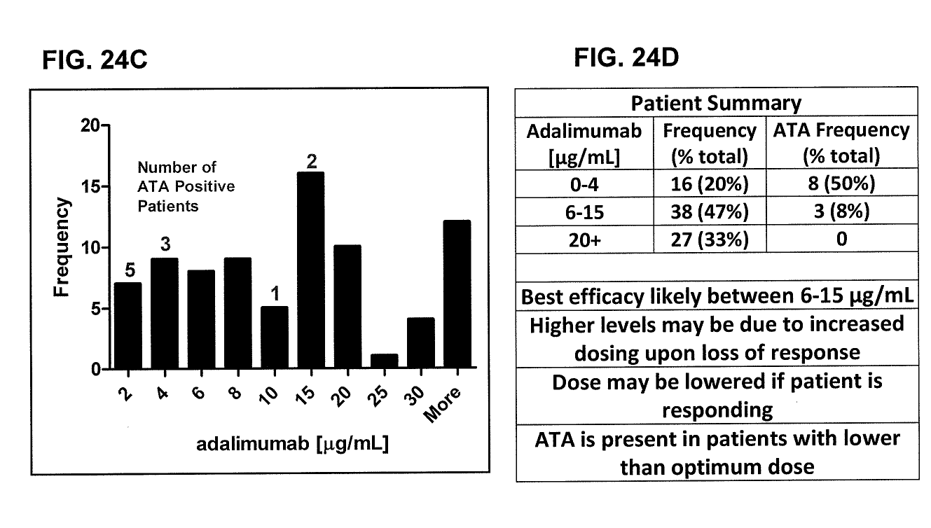

Measurement of Adalimumab and Other Anti-TNF Biologics. The HMSA can be used for analysis of any biological therapeutic and associated anti-drug antibodies, as well as autoantibodies in autoimmune diseases. Rapid development is possible due to robust protocols, high sensitivity, and well established analytical method. See, FIG. 24A-D. The standard curves for each anti-TNF drug are depicted in FIG. 24A. The mean concentration of ADL in patients was 16.5 μg/mL with a range of 1.3-70.9 μg/mL (FIG. 24B). The mean concentration for healthy donors was less than the LLOQ of the assay. A target serum adalimumab concentration of 8 μg/mL has shown clinical utility (Van Assche et al., Gut, 61(2):229-34 (2012)). FIG. 24C shows a histogram of the IBD patients receiving adalimumab therapy in the study. FIG. 24D represents a summary table of the IBD patients tested.

### CONCLUSIONS

Detection of any biologic therapy and associated anti-drug antibodies is possible with the homogeneous mobility shift assay. Therapeutic monitoring of adalimumab and ATA is necessary to ensure patients receive the correct dose of adalimumab. Optimizing dose may improve outcomes and reduce costs. The assay performance is robust and is not affected by serum interferences. This is the only assay method which allows for the detection of ATI in the presence of high levels of IFX, which overcomes the trough sample collection issue. The homogeneous, solution phase mobility shift assay outperforms other solid phase ELISA or equivalent assays and is the method of choice for the measurement of anti-drug antibody and antibody drug in serum. Development of high sensitivity measurement of IFX by HMSA leads to a 30 fold lower cutoff than that of commercially available ELISA assays.

### Example 10

### Influence of Trough Serum Drug Level and Immunogenicity on the Lack of Response to Adalimumab Therapy in IBD Patients

Background:

Anti-TNF-α therapy is effective for the treatment of inflammatory bowel disease (IBD). Nevertheless, over 30% of IBD patients fail to respond to anti-TNF-α therapy and approximately 60% of the patients who respond initially to the therapy will lose the response over time and will need to either dose escalation or switch to another agent to maintain response. Low serum drug levels and/or anti-drug antibody (ADA) generation may play a role for the failure and, recent data suggest monitoring of patients for serum drug and ADA levels is an important strategy for optimal patient management. Here, we report the application of the homogeneous mobility shift assay (HMSA) method for monitoring of adalimumab (ADL) and human antibodies-to-adalimumab (ATA) in serum samples from patients who lost response to ADL treatment.

Methods:

Serum samples were collected from 100 patients who initially responded to ADL therapy for at least three months but were beginning to lose response. ATA and ADL levels in the serum samples were measured by ATA- and ADL-HMSA as described, e.g., in PCT Publication No. WO 2012/154253, U.S. Application Publication No. US 2012/329172, and in U.S. Provisional Application No. 61/683,681, filed Aug. 15, 2012, the disclosures of which are hereby incorporated by reference in their entirety for all purposes, except that in the ATA-HMSA Alexa Fluor 488 labeled ADL (ADL-488) was used as antigen and rabbit anti-ADL serum as standard. Full analytical method validation of both the ATA- and the ADL-HMSA was performed, and cut points for ADL and ATA levels were established with 100 drug-naïve healthy controls. The relationship of the ADL drug level and ATA generation in these patients was analyzed.

Results:

Validation of the ATA- and ADL-HMSA revealed a lower limit of detection to be 0.026 U/mL for ATA and 0.018 μg/mL for ADL in the serum samples. The intra-assay and inter-assay precision determination yielded a coefficient of variation of less than 15%, and the accuracy of the assay is within 20% for both assays. ADL drug tolerance in ATA HMSA is up to 40 μg/ml in the test serum. Serum samples from 100 drug-naïve healthy subjects were tested to set up the cutoff point of 0.55 U/mL (Mean+3.0×SD) for ATA and 0.66 μg/mL for ADL. Analysis of 100 serum samples from patients who were losing response showed that 36% of the patients had an ADL level <3 μg/mL, of these 58.3% were ATA positive. However, only 18% of the patients (4/22) had ATA when their ADL level was over 20 μg/mL. Overall, 40% of the patient (40/100) were positive for ATA.

Conclusions:

Analysis of ADL and ATA levels in non-responding IBD patients showed a high incidence of ATA generation and the ADL levels were inversely correlated with the level of ATA generation. Drug and ADA levels are important determinants of patient response to the therapy.

### Example 11

### Clinical Experience with Measurement of Serum Infliximab and Antibodies to Infliximab Using a New Homogenous Mobility Shift Assay: Results of a Multi-Center Observational Study

Purpose:

To characterize utilization of a new test for monitoring serum infliximab (IFX) and antibodies to infliximab (ATI) in IBD patients on IFX therapy.

Methods:

IBD Patients (pts) undergoing IFX therapy were enrolled in the study if their treating physician determined a need to measure IFX and ATI levels at any time during the course of therapy. Reasons for ordering the test, clinical status, and dosing information were collected at the time of blood draw. IFX and ATI levels were communicated to the physicians within 7 days and actions taken in response to knowing the test were documented. Subjects are followed up at 6 months to determine clinical status.

Results: Baseline data were available for 48 patients (28 CD, 19 UC and 1 indeterminate colitis). Mean age was 48y, mean BMI was 26.4 kg/m² and mean duration of disease was 9.3 years. Forty-two percent of physicians ordered the test because their patients had an inadequate response or loss of response whereas 58% ordered the test as a “baseline measure”. Median IFX concentration was 9.45 μg/mL and 14 of 48 patients (29%) were positive for ATI. Among ATI-positive patients, 86% had IFX concentrations <3 μg/mL, whereas only 9% of ATI-negative patients had IFX concentrations <3 μg/mL (OR=0.02; p=5.05e-7, Fisher's Exact test). Median IFX concentrations were 1 μg/mL and 20 μg/mL in ATI-positive and ATI-negative patients, respectively. Patients with lower disease scores had higher median IFX concentrations (median IFX=9.3 μg/mL for HBI<7 vs. 5.2 μg/mL for HBI<7 in CD and median IFX=34.00 μg/mL for partial Mayo score <6 vs. 14.25 μg/mL for partial Mayo score <6 in UC). In 11 cases (23%), physicians took action in response to knowing the results of IFX/ATI levels including increasing the dose of IFX (n=2), changing dosing interval (n=3), switching to another biologic (n=3) or performing additional work up (n=3). Follow-up data for these patients at 6 months informs the impact of adjusting therapy based on IFX/ATI levels on clinical status.

Conclusions:

Results of this study demonstrate that ATI development is negatively associated with serum IFX concentrations and clinical status. Physicians find the measurement of serum IFX and ATI levels useful in multiple clinical scenarios including inadequate response or loss of response. Physicians made changes to their treatment regimens based on results of IFX/ATI testing in 23% of cases.

### Example 12

### Clinical Experience with Measurement of Serum Infliximab and Antibodies to Infliximab Using a New Homogenous Mobility Shift Assay: Results of a Multi-Center Observational Study

This example illustrates a method of measuring serum levels of IFX and ATI during therapy in IBD patients using a HMSA assay (also known as a MSA assay). This example also illustrates how the method can be used in a clinical setting by physicians to monitor patient's response to IFX therapy. In particular, serum levels of IFX and ATI can be measured in a sample from a IBD patient undergoing IFX therapy at any time during the course of the patient's therapy per a physician's request.

In this study, gastroenterologists obtained samples for IFX/ATI testing from patients undergoing IFX therapy. The physicians were asked to specify whether the reason for requesting the analysis was to establish: 1) “a baseline measurement” in the absence of any therapy concerns, or 2) “a measurement for other purposes” such as, e.g., due to inadequate patient response, disease flare, or autoimmune hypersensitivity reaction. 62% of the samples were ordered to obtain a baseline measurement, while 38% were to obtain “a measurement for other purposes”. In some cases, samples classified in this later group are from patients experiencing inadequate or loss of response to IFX therapy. In other cases, samples in this group are from patients with disease flare or an autoimmune or delayed hypersensitivity reaction to IFX therapy. The physicians recorded patient treatment plans before and after the assay was performed. The effect of test results for IFX and ATI levels on the patients' treatment plans was also evaluated. The results demonstrate that knowledge of IFX and levels can affect a physician's decision in selecting a therapeutic regimen for a patient with IBD including CD and UC.

The sensitivity of the IFX/ATI HMSA assay has a lower limit of detection (LLOD) of 0.1 μg/ml for IFX and LLOD of 3.13 units/ml.

Demographics of the study cohort are presented in Table 3.66% (115/174) of the subjects had CD and 32% (53/115) had UC. 40% (70/174) of the subjects were receiving an IFX dosing of 5 mg/kg weight for 8 weeks. 94% (163/174) of the subjects were in the maintenance phase of the therapy regimen.

TABLE 3
Demographics.

| | Physician's reason for | | | | | |
|---|---|---|---|---|---|---|
| | ordering the test | | | | | |
| | Baseline | | Measurement for | | Combined | |
| | measurement | | other purpose* | | population | |
| | N = 108 | | N = 66 | | N = 174 | |
| Age: yrs, mean (range) | 34.5 | (18-74) | 39.9 | (19-82) | 38 | (18-82) |
| Race: % White | 88.9 | | 92.4 | | 90.2 | |
| Ethnicity: % Jewish | 4.6 | | 7.6 | | 5.7 | |
| BMI kg/m², mean (range) | 26 | (14.9-42.6) | 27.1 | (17.5-54.1) | 26.4 | (14.9-54.1) |
| Duration of disease: yrs, mean | 10.1 | | 10.6 | | 10.3 | |
| CD: # subjects (%) | 70 | (65) | 45 | (68) | 115 | (66) |
| UC: # subjects (%) | 35 | (32) | 21 | (32) | 56 | (32) |

| Dosing at 5 mg/kg per 8 wks: | — | — | 70 | (40) |
|---|---|---|---|---|
| #subjects (%) | | | | |
| Maintenance phase: # subjects (%) | — | — | 163 | (94) |

*Other purposes include: inadequate response, loss of response, disease flare, autoimmune or delayed hypersensitivity reaction.

IFX and ATI levels were measured using a HMSA assay. The median IFX level in all IBD subjects, or restricted to CD or UC subjects are present in Table 4. Analysis revealed that the median IFX is significantly lower in CD subjects who appeared to be responding to IFX (e.g., in the “baseline measurement” group), according to their physicians. CD subjects suspected of having a suboptimal response (e.g., insufficient response, loss of response, disease flare, autoimmune or delayed hypersensitivity response) to IFX (e.g., in the “measurement for other purposes” group) had a higher median IFX. No similar differences were detected in the UC subjects. When the samples were analyzed together as an “all IBD” group, the “baseline measurement” group of subjects had a lower median IFX level (9.85 μg/ml), compared to the “measurement for other purposes” group. The data also shows that the median IFX level is lowest for CD subjects in the combined population (e.g., both “baseline” and “for other purposes” groups), compared to UC subjects and all IBD subjects.

TABLE 4
Median IFX levels.

| | All IBD | | |
|---|---|---|---|
| | Reason for ordering test | | |
| | Baseline | Measurement for | Combined |
| | measurement | other purpose* | population |
| | N = 108 | N = 66 | N = 174 |
| IFX level median (μg/ml) | 9.85 | 12.00 | 11.10 |
| | CD | | |
| | Baseline | Measurement for | Combined |
| | measurement | other purpose* | population |
| | N = 70 | N = 45 | N = 115 |
| IFX level median (μg/ml) | 9.30 | 12.30 | 10.10 |
| | UC | | |
| | Baseline | Measurement for | Combined |
| | measurement | other purpose* | population |
| | N = 35 | N = 21 | N = 56 |
| IFX level median (μg/ml | 11.10 | 11.50 | 11.45 |

*Other purposes include: inadequate response, loss of response, disease flare, autoimmune or delayed hypersensitivity reaction.

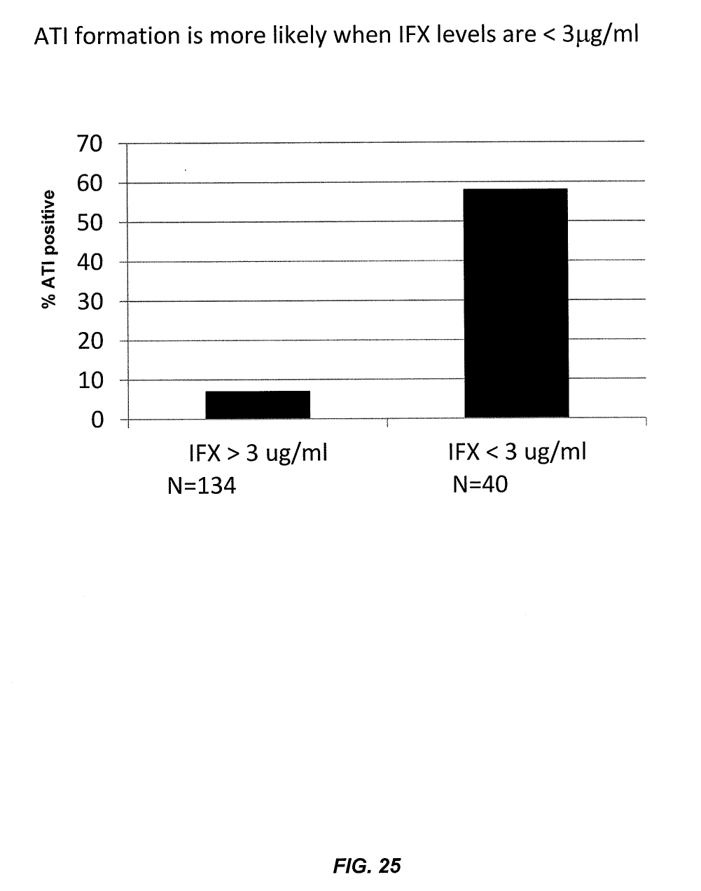

Further analysis revealed that ATI positivity was associated with below threshold levels of IFX. FIG. 25 shows a higher percentage of ATI positive samples had an IFX level of less than 3 μg/ml (e.g., threshold level). Less than 10% of ATI positive samples had IFX levels greater than or equal to 3 μg/ml.

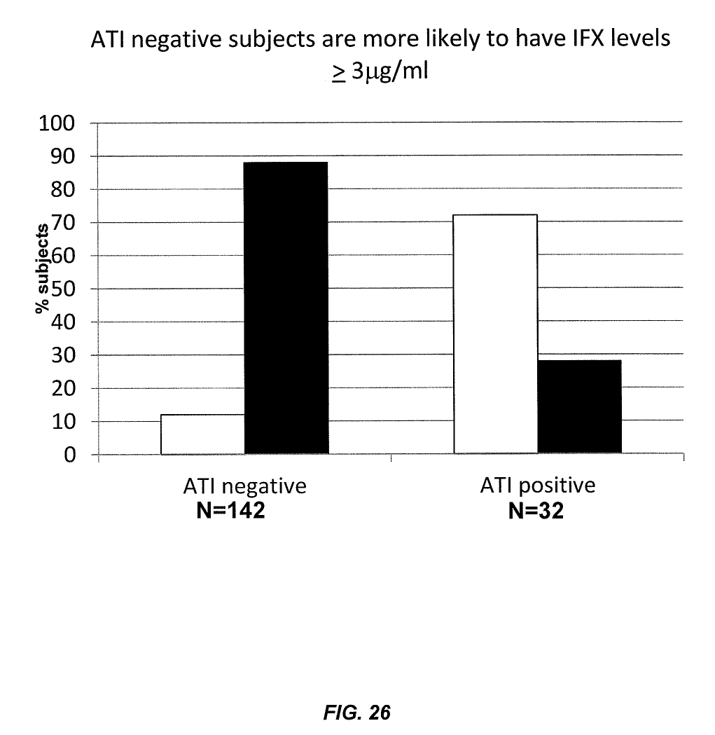

Analysis of ATI negative and ATI positive subjects showed that ATI negative subjects are more likely to have IFX levels greater than or equal to 3 μg/ml (e.g., threshold level). FIG. 26 shows that ATI positivity is correlated to IFX levels less than threshold levels, and ATI negativity is correlated to IFX levels greater than threshold.

Details of the treatment plans of the subjects in the study were evaluated (see, Table 5). Prior to receiving results of IFX/ATI testing, physicians changed the treatment regimen for 9 patients and ordered additional testing for 3 patients. After receiving the results of IFX/ATI testing, physicians changed the treatment regimen for 27 patients and ordered additional testing for 6 patients. There was a 3-fold increase in the physicians' decision to change treatment (e.g., change IFX regimen, add a medication or switch therapeutic biologic) after receiving the testing results, compared to prior to receiving the results.

TABLE 5
Effect of test results for IFX and ATI levels on treatment plans.

| | | Prior to getting | | After getting | |
|---|---|---|---|---|---|
| | | test results | | test results | |
| | Action | (N = 169) | | (N = 173) | |
| Treatment Change | Change IFX Regimen | 6 | (4%) | 17 | (10%) |
| | (increase dose, change | | | | |
| | dosing interval, | | | | |
| | discontinue IFX) | | | | |
| Treatment Change | Add medication | 3 | | 5 | |
| Treatment Change | Switch to different | 0 | | 5 | |
| | biologic | | | | |
| | More tests/procedures | 3 | | 6 | |
| | ordered | | | | |
| | No change/still thinking | 157 | (93%) | 140 | (81%) |

This study showed that IFX and ATI testing can assist physicians in selecting treatment plans for IBD patients. The results show that ATI positivity is associated with below threshold levels of IFX, and that fewer subjects with threshold or above levels of IFX are ATI positive. IFX and ATI testing provided physicians information need to tailor or personalize treatment plans for individual patients. Furthermore, the testing allowed physicians to monitor IFX and ATI levels during the course of therapy.

### Example 13

### Correlation of Trough Serum Drug Level and Immunogenicity on the Lack of Response to Adalimumab Treated Patients

Anti-TNF-α therapy is effective for the treatment of inflammatory disease. Nevertheless, over 30% of patients fail to respond to anti-TNF-α therapy and approximately 60% of the patients who respond initially to the therapy will lose the response over time and will need either dose escalation or switching to another agent to maintain response.

Low serum drug levels and/or anti-drug antibody generation may play a role for the failure and, recent data suggest monitoring of patients for serum drug and anti-drug antibody levels is an important strategy for optimal patient management.

Even though adalimumab is a fully humanized antibody, Antibodies-to-adalimumab (ATA) have been detected in the adalimumab-treated patients with RA, IBD, Psoriatic arthritis and Psoriasis (PS). The incidence of generating ATA in the treated patient is variable, ranging from 6% to 87% using different assay methods.

To overcome the limitations of ELISA assays, we have developed and validated a non-radio labeled, liquid-phase, homogeneous mobility shift assay (HMSA) to measure both antibodies-to-adalimumab (ATA) and adalimumab levels in patients, which is not affected by the presence of high level of drug in the serum.

This assay platform was used to measure adalimumab and ATA levels in patients treated with adalimumab who have lost response.

### 1. ATA-HMSA and Adalimumab HMSA

The procedures for the ATA- and the adalimumab-HMSA are the same as described previously, except that in the ATA-HMSA AlexaFluor 488 labeled adalimumab was used as the antigen and rabbit anti-adalimumab serum was used as the calibration standard for the ATA-HMSA. Briefly, serum samples and calibrators were mixed and incubated with the labeled antigen. The immune complexes formed and the free label were separated and quantitated by a SEC-HPLC system equipped with a fluorescent detector. ChemStation software was used to set up the run and retrieve the data. Analytical validation for both the ATA- and the adalimumab-HMSA were performed based on the standard requirements.

2. Measurement of Adalimumab and ATA in Serum Samples from Patients Treated with Adalimumab

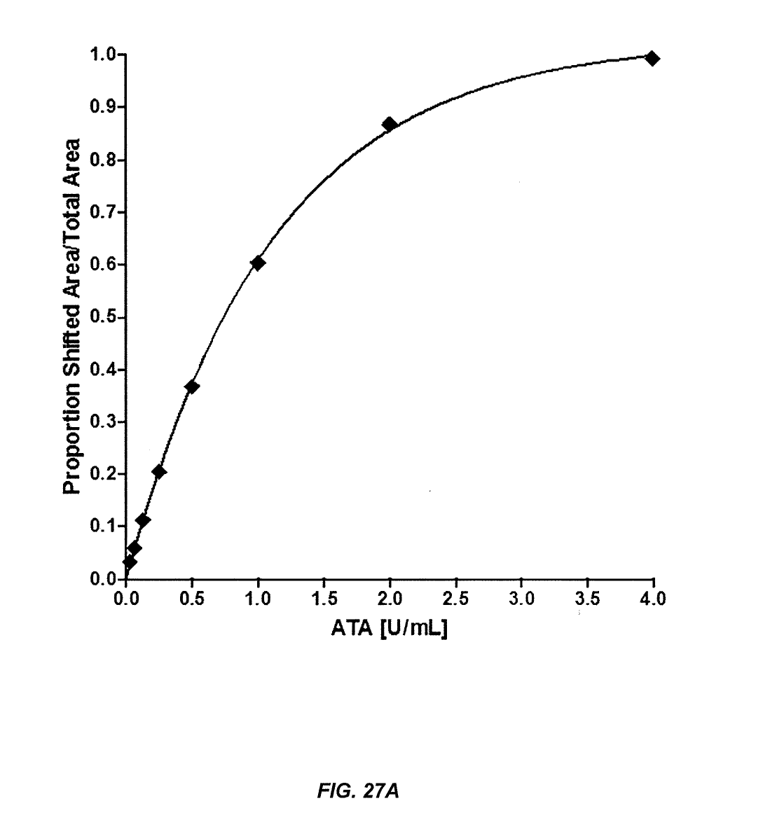

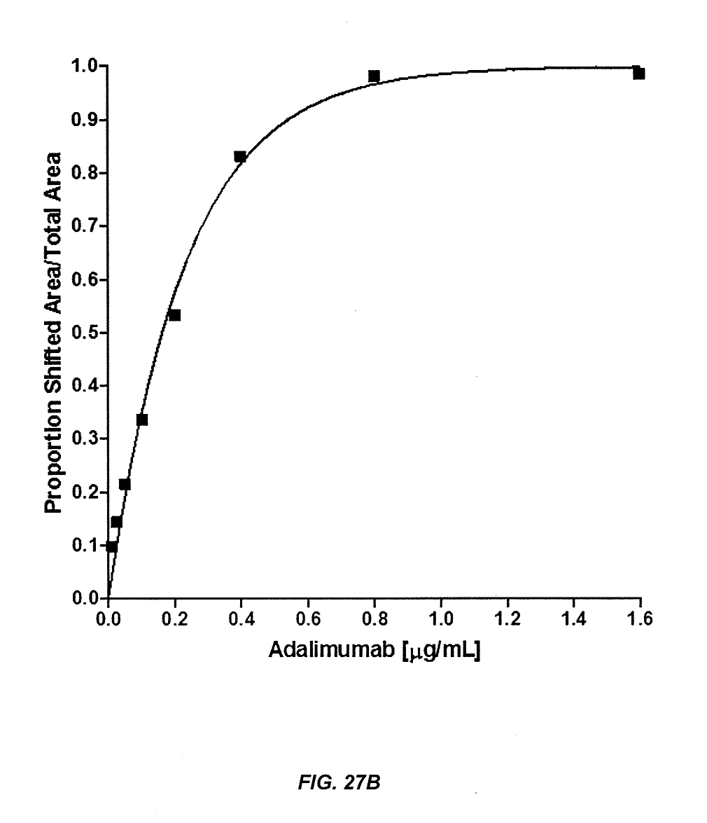

To evaluate the performance of the adalimumab- and the ATA-HMSA in measuring the adalimumab drug and ATA levels in patient serum, we collected serum samples from 100 patients treated with adalimumab. All patients were treated with the adalimumab standard therapy for at least 3 months. These patients initially responded to the therapy but then lost the response based on evaluation of the disease activity indexes for each indication. We measured both ATA and adalimumab concentrations (FIGS. 27A and 27B, respectively) in these serum samples with the HMSA method.

### ATA-HMSA and Adalimumab-HMSA Method Evaluation

The performance characteristics of the ATA-HMSA or adalimumab-HMSA calibration standards, assay limits, assay precision (Intra- and Inter-assay), linearity of dilution and substance interference were evaluated. The R² values and the slopes of each linear regression curve for both assays show linearity.

### 3. Drug Tolerance of the ATA-HMSA.

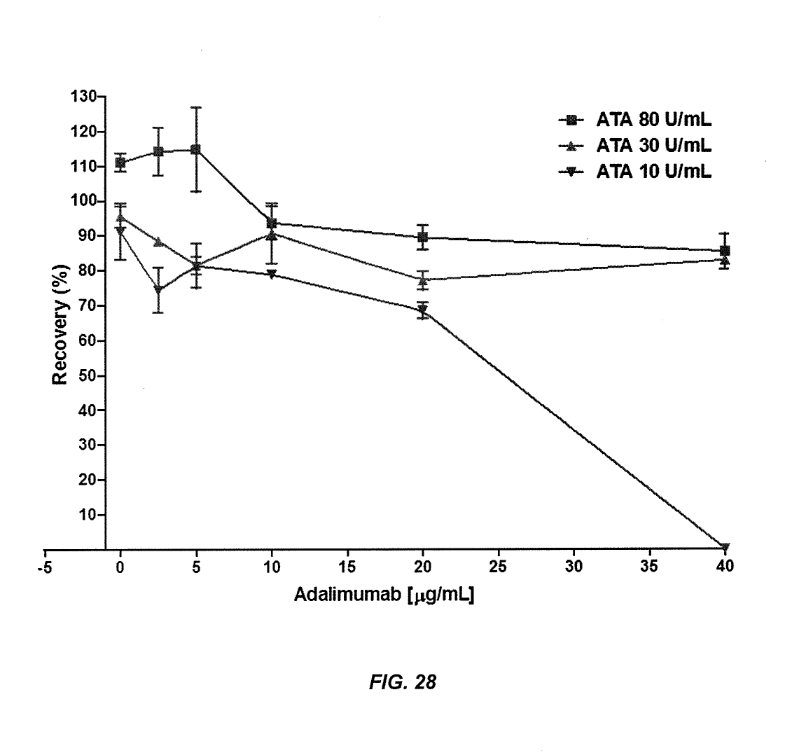

ATA-positive human patient serum was tested in the presence of up to 40 μg/mL adalimumab to determine at what concentrations of adalimumab would interfere with the ATA quantitation. The recovery of the total ATA was calculated as percentage of the original ATA concentration. The ATA-HMSA could detect ATA levels as low as 10 U/mL in the serum samples containing up to 20 μg/mL of adalimumab with 68.5% recovery (FIG. 28).

4. Adalimumab and ATA Levels in Serum Samples from Patients Treated with Adalimumab

An assay cut point was calculated as mean concentration plus 3×SD from 100 healthy controls. The calculated cut point for ATA was 0.549 U/mL. Only one sample from the healthy donors contained ATA levels (0.630 U/mL) at slightly higher than the cut point, which resulted in a clinical specificity of 99%. The calculated cut point for the adalimumab-HMSA was 0.676 μg/mL, which yielded a clinical specificity of 97%.

### 5. Concentrations and ATA Positivity Among IBD, RA and PS Serum Sample

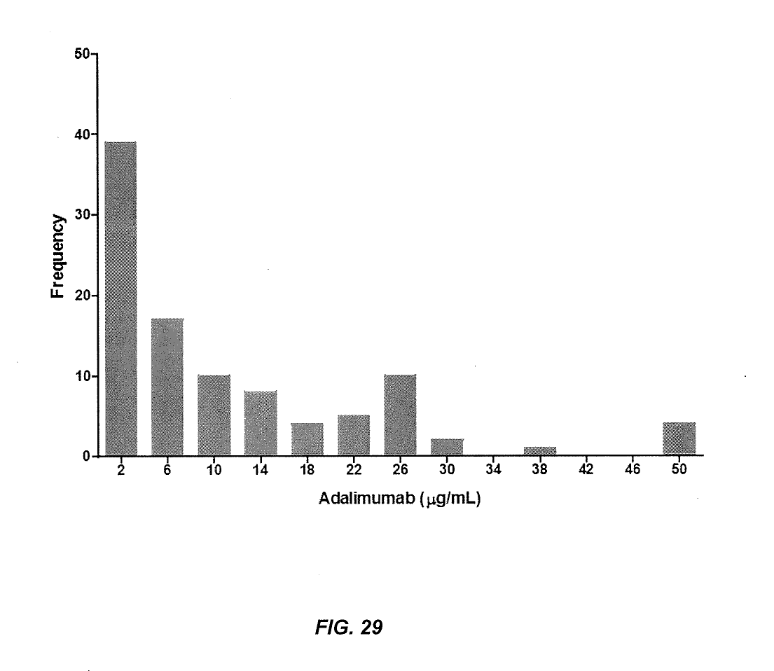

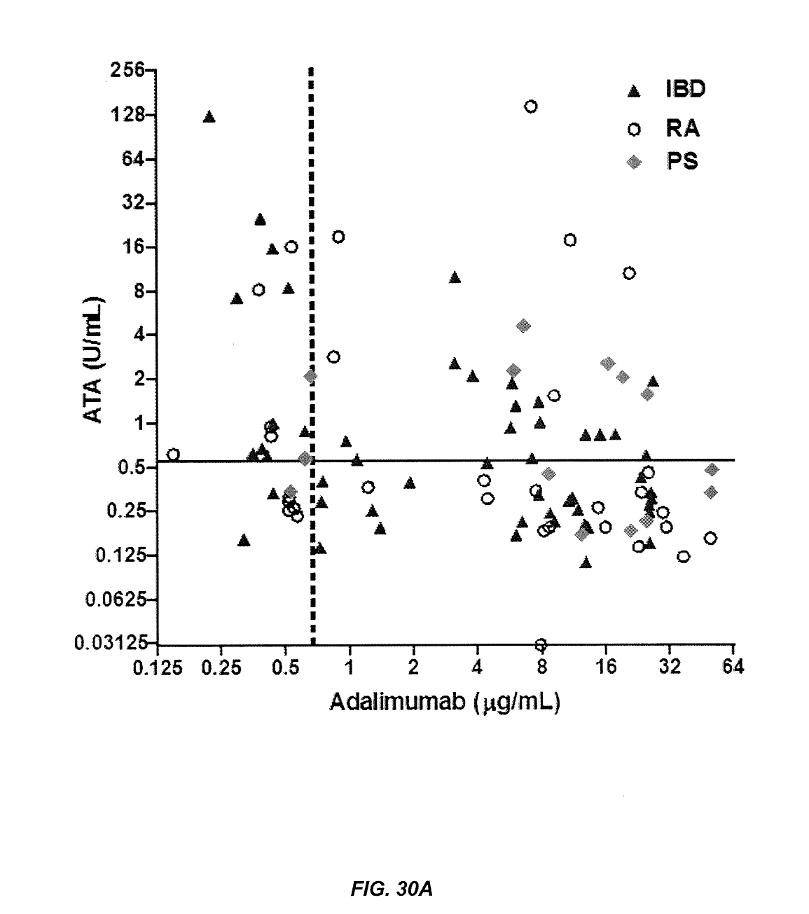

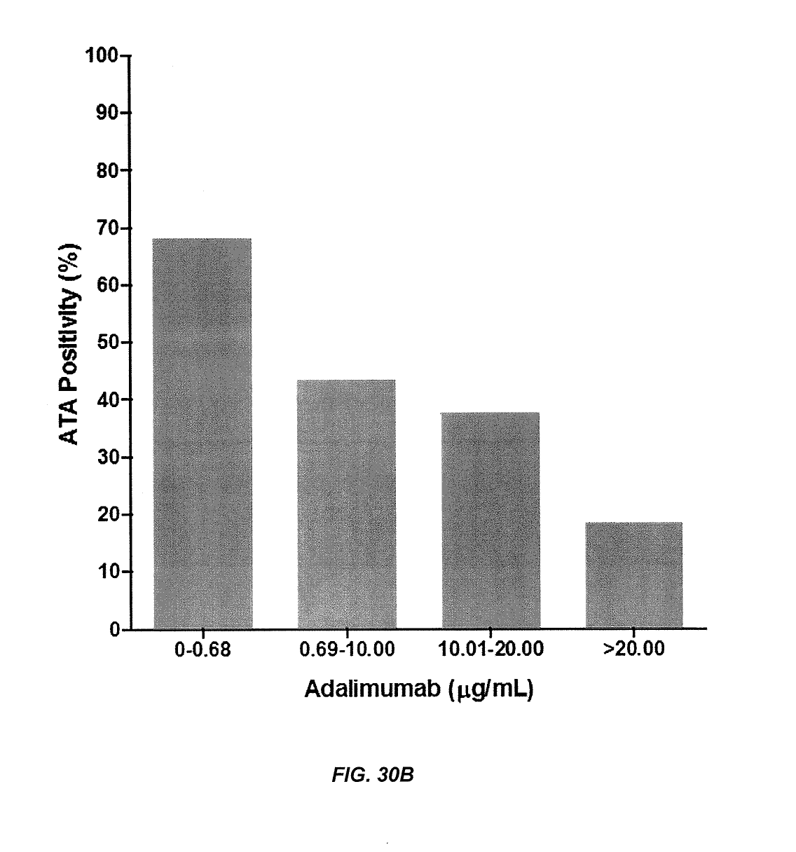

Of the 100 samples tested for adalimumab, 26 samples had drug levels below the cut point of 0.68 μg/mL while 22 samples had the drug levels above 20 μg/mL (FIG. 29). Overall, 44% of the samples were ATA positive and there was an inverse relationship between adalimumab concentration and ATA positivity (FIG. 30A). Among the serum samples in which adalimumab levels were below the cut point, 68% of them were ATA positive. There was no statistically difference on ATA concentrations and ATA positivity among IBD, RA and PS serum sample (FIG. 30B).

A sensitive HMSA for the measurement of ATA and adalimumab in serum was developed and validated. The assays met the performance requirements for both ATA and adalimumab measurements in patient serum.

The assay performance is robust and is not affected by the presence of potential interfering substances in serum. The ATA-HMSA method allows for the detection of ATA in the presence of high levels of adalimumab, which overcomes the trough sample collection issue.

Analysis of adalimumab and ATA levels in non-responding patients showed a high incidence of ATA generation and the adalimumab levels were inversely correlated with the level of ATA generation.

### REFERENCES

W. J. Sandborn, S. B. Hanauer, P. Rutgeerts, R. N. Fedorak, M. Lukas, D. G. MacIntosh, R. Panaccione, D. Wolf, J. D. Kent, B. Bittle, J. Li, P. F. Pollack, Gut 56, (2007) 1232-1239.
G. M. Bartelds, C. L. Krieckaert, M. T. Nurmohamed, P. A. van Schouwenburg, W. F. Lems, J. W. Twisk, B. A. Dijkmans, L. Aarden, G. J. Wolbink, JAMA 305, (2011) 1460-1468.
W. Afif, E. V. Loftus, Jr., W. A. Faubion, S. V. Kane, D. H. Bruining, K. A. Hanson, W. J. Sandborn, Am. J. Gastroenterol. 105, (2010) 1133-1139.
S. L. Wang, L. Ohrmund, S. Hauenstein, J. Salbato, R. Reddy, P. Monk, S. Lockton, N. Ling, S. Singh, J Immunol Methods 382, (2012) 177-188.

### Example 14

### Monitoring of Adalimumab and Antibodies-to-Adalimumab Levels in Patient Serum by the Homogeneous Mobility Shift Assay

### Abstract

Tumor necrosis factor (TNF)-α plays a pivotal role in the pathogenesis of chronic inflammatory diseases. Therapeutic antibodies raised against TNF-α are highly effective in the treatment of chronic inflammatory diseases; however, generation of anti-drug antibodies in anti-TNF-α treated patients is associated with lower serum drug concentrations and loss of clinical response. Therefore, monitoring of patients for serum drug and anti-drug antibody levels is an important strategy for optimal patient management. In this example, we describe the application of the homogeneous mobility shift assay (HMSA) method for the measurement of adalimumab and human antibodies-to-adalimumab (ATA) in serum samples from patients who have lost response to adalimumab treatment.

ATA and adalimumab levels in serum samples were measured using the novel HMSA methodology, in which AlexaFluor-488 labeled adalimumab (adalimumab-488) was used as the antigen for the ATA-HMSA, and rabbit anti-adalimumab serum as the standard. Full analytical method validation of both the ATA- and the adalimumab-HMSA was performed, and cut points for adalimumab and ATA levels were established with 100 drug-naïve healthy controls. Serum samples were collected from 100 patients who had initially responded to adalimumab therapy for at least three months, but were beginning to lose response. The relationship of the adalimumab drug level and ATA generation in these patients was analyzed.

Validation of the ATA- and the adalimumab-HMSA revealed a lower limit of detection to be 0.026 U/mL for ATA and 0.018 μg/mL for adalimumab in serum samples. Intra-assay and inter-assay precision determination yielded a coefficient of variation of less than 15%, and the accuracy of both assays was within 20%. Adalimumab drug tolerance in the ATA-HMSA was up to 20 μg/mL in the test serum. Serum samples from 100 drug-naïve healthy subjects were tested to set-up the cut point of 0.55 U/mL (Mean+3.0×SD) for ATA and 0.68 μg/mL for adalimumab. Analysis of 100 serum samples from patients who were losing response to adalimumab showed that 26% had an adalimumab level below the cut point, of these 68% were ATA positive. However, only 18% of the patients (4/22) had ATA when their adalimumab level was above 20 μg/mL. Overall, 44% of the patients (44/100) were positive for ATA.

Analysis of adalimumab and ATA levels in non-responding patients showed a high incidence of ATA generation, and adalimumab levels were inversely correlated with the level of ATA generation. In conclusion, this example presents evidence that drug and anti-drug antibody levels are important determinants of patient response to therapy.

Abbreviations: ATA, antibodies-to-adalimumab; CD, Crohn's disease; HMSA, homogenous mobility shift assay; IBD, inflammatory bowel disease; PS, psoriasis; RA, rheumatoid arthritis; TNF-α, tumor necrosis factor-alpha; UC, ulcerative colitis.

### 1. Introduction

Many chronic inflammatory diseases are mediated by up-regulation of the pro-inflammatory cytokine tumor necrosis factor-alpha (TNF-α) (Suryaprasad et al., The biology of TNF blockade, Autoimmun. Rev. 2 346-357 (2003); Kopylov et al., Clinical utility of antihuman lambda chain-based enzyme-linked immunosorbent assay (ELISA) versus double antigen ELISA for the detection of anti-infliximab antibodies, Inflamm Bowel Dis. 18 1628-1633 (2012); Sandborn et al., Reinduction with certolizumab pegol in patients with relapsed Crohn's disease: results from the PRECiSE 4 Study, Clin Gastroenterol Hepatol. 8 696-702 (2010)). The therapeutic use of anti-TNF-α antagonists such as infliximab, adalimumab, and certolizumab pegol has greatly improved the treatment of rheumatoid arthritis (RA), psoriasis (PS), and inflammatory bowel disease (IBD, Crohn's disease and/or ulcerative colitis). The anti-TNF-α therapeutics are effective in reducing disease activity, and offer significant benefits in quality of life and may have the potential to change the progression of the disease when given early (Tracey et al., Tumor necrosis factor antagonist mechanisms of action: a comprehensive review, Pharmacol Ther. 117 244-279 (2008); Magro et al., Management of inflammatory bowel disease with infliximab and other anti-tumor necrosis factor alpha therapies, BioDrugs. 24 Suppl 1 3-14 (2010)). However, over 30% of patients fail to respond to anti-TNF-α therapy, and approximately 60% of patients who responded initially lose the response over time, and require either drug dose-escalation or switch to an alternative agent in order to maintain response (Hanauer et al., Maintenance infliximab for Crohn's disease: the ACCENT I randomised trial, Lancet. 359 1541-1549 (2002); Gisbert et al., Loss of response and requirement of infliximab dose intensification in Crohn's disease: a review, Am J. Gastroenterol. 104 760-767 (2009); Regueiro et al., Infliximab dose intensification in Crohn's disease, Inflamm Bowel Dis. 13 1093-1099 (2007)). Anti-drug antibody formation may increase drug clearance in treated patients and/or neutralize the drug effect, thereby potentially contributing to the loss of response (Miheller et al., Anti-TNF trough levels and detection of antibodies to anti-TNF in inflammatory bowel disease: are they ready for everyday clinical use?, Expert Opin. Biol Ther. 12 179-192 (2012)). Anti-drug antibodies could also cause adverse events such as serum sickness and hypersensitivity reactions (Brennan et al., Safety and immunotoxicity assessment of immunomodulatory monoclonal antibodies, MAbs. 2 233-255 (2010); Emi, J. F. de Carvalho, S. C. Artur Almeida, E. Bonfa, Immunogenicity of Anti-TNF-alpha agents in autoimmune diseases, Clin Rev Allergy Immunol. 38 82-89 (2010)). Moreover, recent data suggest that the standard dosing regimen for TNF-α-blocking drugs may be suboptimal in some IBD patients, and an individualized dosing regimen to achieve therapeutic drug levels may be needed in order to maximize the initial drug response and to maintain remission (Colombel et al., Therapeutic drug monitoring of biologics for inflammatory bowel disease, Inflamm Bowel Dis. 18 349-358 (2012)). Therefore, accurate monitoring of serum drug and anti-drug antibody levels should be an important part of therapy for patients being treated with protein-based drugs. For this purpose, we have previously developed and validated a novel homogeneous mobility shift assay (HMSA) using size-exclusion high-performance liquid chromatography (SE-HPLC) to quantitatively measure both infliximab and antibody-to-infliximab (ATI) in human serum (Wang et al., Development and validation of a homogeneous mobility shift assay for the measurement of infliximab and antibodies-to-infliximab levels in patient serum, J Immunol Methods. 382 177-188 (2012)). The HMSA method overcomes many limitations found in other methods, such as the bridging enzyme-linked immunosorbent assay (ELISA) method (Baert et al., Influence of immunogenicity on the long-term efficacy of infliximab in Crohn's disease, N. Engl. J Med. 348 601-608 (2003)), and the radioimmunoassay (RIA) method (Aarden et al., Immunogenicity of anti-tumor necrosis factor antibodies-toward improved methods of anti-antibody measurement, Curr. Opin. Immunol. 20 431-435 (2008)). The advantages of the HMSA method include high sensitivity, specificity, and accuracy and the ability to detect all isotypes of immunoglobulin and subtypes of IgG such as IgG₄. Using the HMSA method, it is possible to measure ATI in the presence of high levels of infliximab drug in patient serum (Wang et al., Development and validation of a homogeneous mobility shift assay for the measurement of infliximab and antibodies-to-infliximab levels in patient serum, J Immunol Methods. 382 177-188 (2012)), which is not possible with the ELISA method.

Adalimumab (Humira®) is a fully humanized monoclonal antibody against TNF-α, and is approved for the treatment of RA and Crohn's disease (CD) via subcutaneous injection. In the CLASSIC I and II clinical trials, adalimumab treatment resulted in a significantly higher rate of remission of CD (Hanauer et al., Human anti-tumor necrosis factor monoclonal antibody (adalimumab) in Crohn's disease: the CLASSIC-I trial, Gastroenterology, 130 323-333 (2006); Sandborn et al., Adalimumab for maintenance treatment of Crohn's disease: results of the CLASSIC II trial, Gut. 56 1232-1239 (2007)). Even though it is fully humanized, adalimumab does not eliminate the risk of immunogenicity in both CD and RA patients (Karmiris et al., Influence of trough serum levels and immunogenicity on long-term outcome of adalimumab therapy in Crohn's disease, Gastroenterology. 137 1628-1640 (2009); Bartelds et al., Development of antidrug antibodies against adalimumab and association with disease activity and treatment failure during long-term follow-up, JAMA. 305 1460-1468 (2011)). Generation of antibodies-to-adalimumab (ATA) in the serum is associated with lower serum adalimumab concentrations and reduced response rate to treatment. In the present study, we evaluated the feasibility of using the HMSA method for the measurement of adalimumab and ATA levels in patients who have lost response to adalimumab therapy.

### 2. Materials and Methods

### 2.1. Materials

Individual serum samples from healthy controls were obtained from blood bank donors (Golden West Biologics, Temecula, Calif.). Sera from patients with RA, PS, and IBD treated with adalimumab were drawn according to a protocol approved by an Institutional Review Board/Ethics Committee. Unless otherwise noted, all reagents and chemicals were obtained from Thermo Fisher Scientific (Waltham, Mass.) or Sigma Aldrich Corporation (St. Louis, Mo.).

### 2.2. Preparation of Reagents

### 2.2.1. ATA Calibration Serum

ATA-positive sera were prepared by immunizing two rabbits with purified adalimumab

(ProSci, Inc., San Diego, Calif.). Bleeds of anti-adalimumab positive sera from the rabbits were pooled and the relative amount of ATA was arbitrary defined as 100 U/mL, equal to 1:100 dilutions. The pooled ATA calibration serum was aliquoted and stored at −70° C.

### 2.2.2. Conjugation of Adalimumab and TNF-α

The method for the conjugation of AlexaFluor-488 to adalimumab was same as described previously (Wang et al., Development and validation of a homogeneous mobility shift assay for the measurement of infliximab and antibodies-to-infliximab levels in patient serum, J Immunol Methods. 382 177-188 (2012)). Briefly, commercially-available adalimumab (Humira®, Abbott Laboratories, Abbott Park, Ill.) was buffer exchanged with phosphate buffered saline (PBS, pH 7.3) and labeled with AlexaFluor-488 (Life Technology, Carlsbad, Calif.) following the manufacturer's instructions. Only those conjugates containing 2 to 3 fluorescent dyes per antibody qualified for the ATA-HMSA. Conjugation of AlexaFluor-488 to TNF-α was performed as described previously (Wang et al., Development and validation of a homogeneous mobility shift assay for the measurement of infliximab and antibodies-to-infliximab levels in patient serum, J Immunol Methods. 382 177-188 (2012)).

### 2.3. HMSA for ATA and Adalimumab

The procedure for the ATA-HMSA and the adalimumab-HMSA were similar to the ATI-HMSA as described previously (Wang et al., Development and validation of a homogeneous mobility shift assay for the measurement of infliximab and antibodies-to-infliximab levels in patient serum, J Immunol Methods. 382 177-188 (2012)), except that AlexaFluor-488 labeled adalimumab was used in the ATA-HMSA. In brief, serum samples were first acid dissociated with 0.5 M citric acid (pH 3.0) for 1 hour at RT, and then neutralized with 10×PBS (pH 7.3) in the presence of adalimumab-AlexaFluor-488 in a 96-well plate format. The plate was incubated for 1 hour at RT on an orbital shaker to complete the formation of the immune complexes. The equilibrated samples were filtered through a MultiScreen-Mesh Filter plate equipped with a Durapore membrane (0.22 μm; EMD Millipore, Billerica, Mass.) into a 96-well receiver plate (Nunc, Thermo Fisher Scientific, Waltham, Mass.). The recovered solutions were individually loaded into an HPLC system (Agilent Technologies 1200 series HPLC system, Santa Clara, Calif.) equipped with a BioSep SEC-3000 column (Phenomenex, Torrance, Calif.). The chromatography was run at the flow-rate of 1 mL/min with 1×PBS (pH 7.3) as the mobile phase for a total of 20 min, and was monitored with a fluorescence detector at excitation and emission wavelengths of 494 nm and 519 nm, respectively. ChemStation Software (Agilent Technologies, Santa Clara, Calif.) was used to set-up and collect data from the runs automatically and continuously.

To generate a standard curve, one aliquot of the stock ATA calibration serum was thawed and diluted to 2% in volume with rabbit serum (Sigma Aldrich, St. Louis, Mo.) in HPLC assay buffer (1×PBS, pH 7.3) to achieve final concentrations in the assay wells of 0.031, 0.063, 0.125, 0.250, 0.500, 1.000, 2.000, and 4.000 U/mL. Three quality control (QC) samples were prepared by diluting the calibration serum in assay buffer with 0.1% BSA to yield the high (1.600 U/mL), mid (0.600 U/mL), and low (0.200 U/mL) control concentrations. Similarly, adalimumab calibration standards were prepared by serially diluting purified adalimumab with assay buffer containing 0.1% BSA to achieve final concentrations of 0.013, 0.025, 0.050, 0.100, 0.200, 0.400, 0.800 and 1.600 μg/mL of adalimumab and final NHS concentration of 4% in the reaction mixture. Three adalimumab QC samples were prepared by diluting the adalimumab calibration standard with assay buffer and 0.1% BSA to yield the high (25 μg/mL), mid (10 μg/mL), and low (5 μg/mL) control concentrations.

### 2.4. ATA-HMSA and Adalimumab-HMSA Evaluation

The analytical validations including the performance characteristics for the ATA-HMSA and the adalimumab-HMSA (calibration standards, assay limits, assay precision [intra- and inter-assay], linearity of dilution, and substance interference) were performed based on the industrial recommendations (Shankar et al., Recommendations for the validation of immunoassays used for detection of host antibodies against biotechnology products, J Pharm Biomed. Anal. 48 1267-1281 (2008)). A panel of serum samples from drug-naïve healthy donors (n=100; Golden West Biologics. Temecula, Calif.) were analyzed to determine the assay cut points for the ATA-HMSA and adalimumab-HMSA. The assay cut points were defined as the threshold above which samples were deemed to be positive, and was set to have an upper negative limit of approximately 99%, calculated by using the lowest mean value of individual samples interpolated from the standard curve+3.0× the standard deviation (SD).

### 2.5. Data Analysis

Data analysis was performed with the use of a proprietary automated program run on R software (R Development Core Team, Vienna, Austria) (Wang et al., Development and validation of a homogeneous mobility shift assay for the measurement of infliximab and antibodies-to-infliximab levels in patient serum, J Immunol Methods. 382 177-188 (2012)). Briefly, the R program opened the ChemStation files, normalized the spectra, determined the area under each peak, and calculated the proportion of total peak areas shifted to the bound ATA/adalimumab-AlexaFluor-488 complexes over the total bound and free adalimumab-AlexaFluor-488 peak areas in the ATA-HMSA. An exponential association standard curve was generated from the standards and the measured ATA values were interpolated from the curve. To obtain the actual ATA and adalimumab concentrations in the serum, the interpolated results from the standard curves were multiplied by the dilution factor.

### 3. Results

### 3.1. Evaluation of the ATA-HMSA and the Adalimumab-HMSA

Because the maintenance protocol for patients treated with adalimumab requires biweekly dosing, and the estimated half-life of the drug in human blood is 15-20 days, the collection of a large quantity of ATA-positive sera from patients for use as calibration standards is a challenge. In theory, antisera from any mammalian species will bind to and form immune complexes with adalimumab, and show a similar SE-HPLC profile when compared to human immune complexes. Therefore, in order to produce a large quantity of ATA-positive sera for calibration needs, two rabbits were immunized with purified adalimumab and different bleeds of antisera were pooled to serve as calibration standards. The relative amount of ATA was arbitrarily defined as 100 U/mL, equal to 1:100 dilutions. When serial dilutions of the ATA calibration standards were incubated with adalimumab-AlexaFluor-488, dose-dependent immune complexes were formed with concomitant reduction of the free adalimumab-AlexaFluor-488. Analyses were then conducted by SE-HPLC, in which the shifted peak area of the immune complexes and free adalimumab-AlexaFluor-488 were calculated. The standard curve generated by plotting the proportion shifted area vs. ATA concentration is shown in FIG. 27A. The lowest concentration of ATA in the standard curve was 0.031 U/mL. The error for the back-calculated values of the 29 standard curve runs was within 10%, except for the lowest concentration (0.031 U/mL) (Table 6). The CV was <20% for concentrations above 0.031 U/mL, and the dynamic range of the assay was two orders of magnitude. The calculated limit of detection (LOD) was 0.026 U/mL from the 29 standard curve runs based on the method described previously (Wang et al., Development and validation of a homogeneous mobility shift assay for the measurement of infliximab and antibodies-to-infliximab levels in patient serum, J Immunol Methods. 382 177-188 (2012)). The calculated lower limit of quantitation (LLOQ) and upper limit of quantitation (ULOQ) were 0.063 U/mL and 25.000 U/mL, respectively. Complete analytical validation of the ATA-HMSA was performed with the high (1.600 U/mL), mid (0.600 U/mL), and low (0.200 U/mL) QCs by multiple analysts using different instruments on different days. As shown in Table 7, the ATA-HMSA intra-assay precision had a CV<3% and an accuracy rate was <13% error. The inter-assay precision (run-to-run, analyst-to-analyst and instrument-to-instrument) had a CV of <9% and an accuracy of <18% error.

To evaluate the performance of the adalimumab-HMSA, purified adalimumab was used as the calibration standard and AlexaFluor-488 was used to label the TNF-α. The performance characteristics of the adalimumab-HMSA standard curve in the concentration range of 0.012 μg/mL to 1.600 μg/mL (FIG. 27B) was similarly assessed over 29 experimental runs by multiple analysts using different instruments on different days (Table 8). The error for the back-calculated value of the 29 standard curve runs was within 15% except for the highest and lowest concentrations (Table 6). The CV was <25% except for the lowest concentration, and the dynamic range was two orders of magnitude. The calculated LOD, LLOQ, and ULOQ for the adalimumab-HMSA were 0.018 μg/mL, 0.040 μg/mL, and 1.100 μg/mL, respectively. As shown in Table 8, the intra-assay precision and accuracy for the adalimumab-HMSA were <20% and <3%, respectively, whereas the inter-assay precision and accuracy for the adalimumab-HMSA were <12% and <22%, respectively.

To determine the linearity of dilution of both the ATA-HMSA and the adalimumab-HMSA, human serum samples containing a high titer of ATA or a high concentration of adalimumab were used. The samples were diluted serially 2-fold within the linear range of the standard curves and analyzed using the ATA-HMSA and the adalimumab-HMSA, respectively. The observed values of ATA or adalimumab were compared with the expected levels of ATA or adalimumab in the serum samples. The R² values and the slopes of each linear regression curve for both assays show linearity.

ATA-positive human patient serum was tested in the presence of up to 40 μg/mL adalimumab to determine at what concentrations of adalimumab would interfere with the ATA quantitation. The recovery of the total ATA was calculated as percentage of the original ATA concentration. The ATA-HMSA could detect ATA levels as low as 10 U/mL in the serum samples containing up to 20 μg/mL of adalimumab with 68.5% recovery (FIG. 28).

The effect of the potential interfering substances present in the serum was also evaluated in both the ATA-HMSA and the adalimumab-HMSA as described previously (Wang et al., Development and validation of a homogeneous mobility shift assay for the measurement of infliximab and antibodies-to-infliximab levels in patient serum, J Immunol Methods. 382 177-188 (2012)). No significant interference was observed in the physiological levels of TNF-α, TNF-13, sTNFR₁, sTNFR₂, immunoglobulin, rheumatoid factor, hemolyzed serum, and lipemic serum (data not shown). In addition, the presence of azathioprine up to 10 μM and methotrexate up to 2.0 mM did not affect the assays.

TABLE 6
Characteristics of the ATA-HMSA and the adalimumab-HMSA standard curves.

| ATA-HMSA | | | | | Adalimumab-HMSA | | | | |
|---|---|---|---|---|---|---|---|---|---|
| | Back | | | | | Back | | | |
| ATA | Calculated | | | | Adalimumab | Calculated | | | |
| Standard | Concentration | Error | CV | | Standard | Concentration | Error | CV | |
| (U/mL) | (Mean, U/mL) | (%) | (%) | n | (μg/mL) | (Mean, μg/mL) | (%) | (%) | n |
| 4.000 | 3.820 | 4.51 | 0.47 | 29 | 1.600 | 0.973 | 39.17 | 0.31 | 29 |
| 2.000 | 2.099 | 4.94 | 4.87 | 29 | 0.800 | 0.898 | 12.21 | 4.89 | 29 |
| 1.000 | 0.970 | 3.03 | 7.76 | 29 | 0.400 | 0.405 | 1.35 | 10.36 | 29 |
| 0.500 | 0.496 | 0.71 | 9.31 | 29 | 0.200 | 0.192 | 3.94 | 13.41 | 29 |
| 0.250 | 0.263 | 5.16 | 10.45 | 29 | 0.100 | 0.104 | 4.12 | 16.09 | 29 |
| 0.125 | 0.132 | 5.58 | 15.8 | 29 | 0.050 | 0.055 | 9.39 | 18.92 | 29 |
| 0.063 | 0.062 | 1.66 | 19.73 | 29 | 0.025 | 0.026 | 2.46 | 24.93 | 29 |
| 0.031 | 0.017 | 44.84 | 29.03 | 29 | 0.012 | 0.006 | 51.84 | 34.58 | 29 |

TABLE 7
Assay precision of the ATA-HMSA.

| | | | | Inter-Assay Precision | | | | | | | | |
|---|---|---|---|---|---|---|---|---|---|---|---|---|
| | Intra-Assay Precision | | | Run-to-Run | | | Analyst-to-Analyst | | | Instrument-to-Instrument | | |
| | (n = 10) | | | (n = 5) | | | (n = 3) | | | (n = 3) | | |
| | High | Mid | Low | High | Mid | Low | High | Mid | Low | High | Mid | Low |
| Expected | 1.600 | 0.600 | 0.200 | 1.600 | 0.600 | 0.200 | 1.600 | 0.600 | 0.200 | 1.600 | 0.600 | 0.200 |
| (U/mL) | | | | | | | | | | | | |
| Measured | 1.800 | 0.574 | 0.180 | 1.797 | 0.562 | 0.176 | 1.689 | 0.532 | 0.169 | 1.708 | 0.526 | 0.166 |
| (Mean, | | | | | | | | | | | | |
| U/mL) | | | | | | | | | | | | |
| SD | 0.039 | 0.009 | 0.016 | 0.125 | 0.024 | 0.012 | 0.142 | 0.040 | 0.011 | 0.123 | 0.031 | 0.010 |
| CV (%) | 2.170 | 1.600 | 1.830 | 6.94 | 4.33 | 6.96 | 8.420 | 7.450 | 6.530 | 7.190 | 5.950 | 5.970 |
| Accuracy | 12.530 | 4.410 | 10.250 | 12.33 | 6.41 | 12.12 | 5.590 | 11.250 | 15.560 | 6.750 | 12.370 | 17.180 |
| (% Error) | | | | | | | | | | | | |

TABLE 8
Assay precision of the adalimumab-HMSA.

| | | | | Inter-Assay Precision | | | | | | | | |
|---|---|---|---|---|---|---|---|---|---|---|---|---|
| | Intra-Assay Precision | | | Run-to-Run | | | Analyst-to-Analyst | | | Instrument-to-Instrument | | |
| | (n = 10) | | | (n = 5) | | | (n = 3) | | | (n = 3) | | |
| | High | Mid | Low | High | Mid | Low | High | Mid | Low | High | Mid | Low |
| Expected | 1.000 | 0.400 | 0.200 | 1.000 | 0.400 | 0.200 | 1.000 | 0.400 | 0.200 | 1.000 | 0.400 | 0.200 |
| (μg/mL) | | | | | | | | | | | | |
| Measured | 0.974 | 0.392 | 0.189 | 1.016 | 0.446 | 0.215 | 1.032 | 0.487 | 0.220 | 0.917 | 0.442 | 0.202 |
| (Mean, | | | | | | | | | | | | |
| μg/mL) | | | | | | | | | | | | |
| SD | 0.017 | 0.075 | 0.019 | 0.052 | 0.052 | 0.021 | 0.043 | 0.024 | 0.013 | 0.056 | 0.007 | 0.010 |
| CV (%) | 1.700 | 19.200 | 1.700 | 5.060 | 11.700 | 9.680 | 4.170 | 4.820 | 5.990 | 6.120 | 1.650 | 4.920 |
| Accuracy | 2.620 | 2.090 | 2.620 | 1.650 | 11.570 | 7.430 | 3.140 | 21.670 | 10.200 | 8.270 | 10.540 | 1.270 |
| (% Error) | | | | | | | | | | | | |

### 3.2. Adalimumab Drug Tolerance of the ATA-HMSA

Substantial concentrations of adalimumab may be present in the serum from patients, even if the blood is drawn at the trough time point due to the biweekly dosing regimen. As discussed previously, the presence of therapeutic antibody in the patient serum significantly affected the quantitative measurement of anti-drug antibodies in the bridging ELISA assay. To address this issue in the HMSA-based assays, ATA-positive human patient serum was tested in the presence of up to 40 μg/mL adalimumab to determine the concentration at which adalimumab would interfere with the ATA quantitation. When adalimumab was present in a sample, the total ATA was calculated using the equilibrium equation: A+B+C=AC+BC, where A=unlabeled adalimumab, B=labeled adalimumab, and C=ATA.

In this equation the following values were known for each sample: A was the concentration obtained from performing the adalimumab-HMSA. B was the known amount of adalimumab-AlexaFluor-488 spiked into the sample. BC was the concentration of the ATA/adalimumab-AlexaFluor-488 complex determined from the ATA-HMSA.

Knowing that the sample was acid dissociated before and allowed to reach equilibrium, then: BC/B=AC/A

Solving for AC, the concentration of ATA bound to unlabeled adalimumab was calculated. Therefore, the total ATA in the sample was equal to AC+BC.

$$\begin{matrix}ATA bound to \\ unlabeled \\ adalimumab\end{matrix} = \frac{\begin{matrix}U \text{/} mL ATA from \\ mobility shift assay\end{matrix} \times \begin{matrix}µg unlabeled \\ adalimumab\end{matrix}}{µg labeled adalimumab}$$

As shown in FIG. 27, the ATA-HMSA could detect ATA levels as low as 10 U/mL in serum samples containing 20 μg/mL of adalimumab with 68.5% recovery.

### 3.3. Cut Point Determinations for the ATA-HMSA and the Adalimumab-HMSA

We screened 100 serum samples collected from adalimumab drug-naïve healthy subjects to establish the cut points for the ATA-HMSA and the adalimumab-HMSA. In the ATA-HMSA, the proportion of shifted area over the total area was near the LOD, and the mean value of the extrapolated ATA concentrations in serum from the standard curve (multiplied by the dilution factor of 50) was 0.329±0.073 U/mL. The calculated cut point for ATA was 0.549 U/mL (mean+3.0×SD). There was only one sample that contained ATA levels slightly higher than the cut point (0.630 U/mL), which resulted in a clinical specificity of 99%. The same method was applied to calculate the cut point for the adalimumab-HMSA: 0.676 μg/mL, with a clinical specificity of 97%.

3.4. Measurement of Adalimumab and ATA in Serum Samples from Patients Treated with Adalimumab

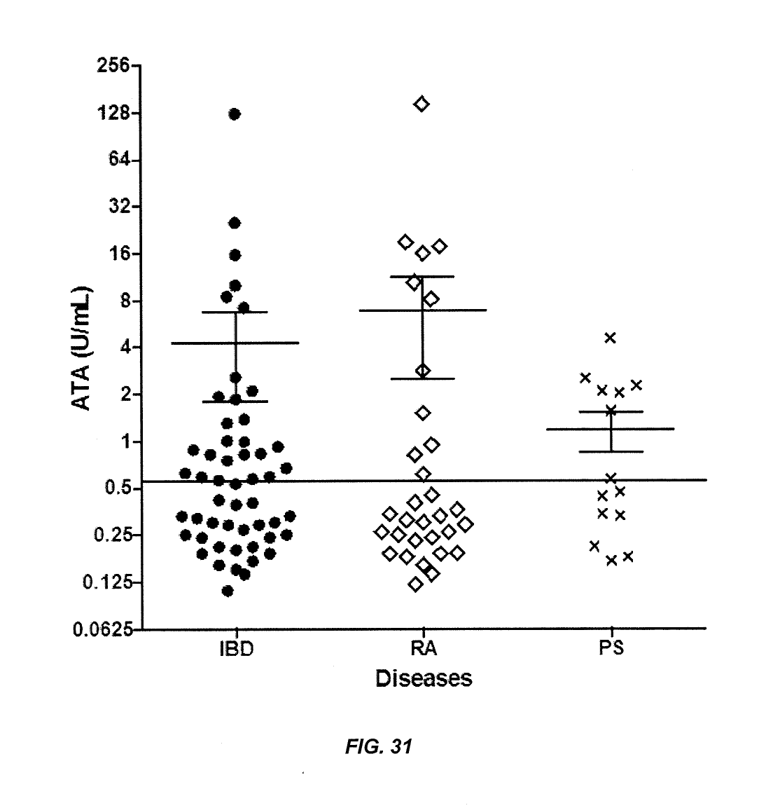

Recent scientific publications have reported that the incidence of ATA in adalimumab-treated patients varies from 6% to 87% based on different assay methods (Sandborn et al., Adalimumab for maintenance treatment of Crohn's disease: results of the CLASSIC II trial, Gut. 56 1232-1239 (2007); Bartelds et al., Development of antidrug antibodies against adalimumab and association with disease activity and treatment failure during long-term follow-up, JAMA. 305 1460-1468 (2011); Afif et al., Clinical utility of measuring infliximab and human anti-chimeric antibody concentrations in patients with inflammatory bowel disease, Am. J. Gastroenterol. 105 1133-1139 (2010)). To evaluate the performance of the adalimumab-HMSA and the ATA-HMSA in measuring adalimumab drug and ATA levels in patient serum, we collected serum samples from 100 patients treated with adalimumab. The basic characteristics of the patients are shown in Table 9. All patients were treated with the adalimumab standard therapy for at least 3 months. All patients initially responded to therapy but then lost the response based on evaluation of the disease activity indices for each indication. Adalimumab and ATA concentrations in these serum samples were measured with the HMSA method. All samples were diluted 25-fold for the adalimumab test and 50-fold for the ATA test. If the results of the test were above the ULOQ, the samples were retested with further dilutions in order to obtain accurate results. Of the 100 samples tested for adalimumab, 26 samples had drug levels below the cut point of 0.68 μg/mL, while 22 samples had the drug levels above 20 μg/mL. The distribution of the adalimumab levels in these 100 patients is shown in FIG. 29. The mean average ATA levels in the studied samples were 4.644±19.203 U/mL (mean±SD, n=100), significantly higher than the healthy controls (0.329±0.073 U/mL, mean±SD, n=100, P<0.00001). When the cut point of 0.55 U/mL was applied to the ATA-HMSA test, 44 samples were determined to be ATA positive (44%). The relationship between adalimumab drug concentrations and ATA levels in the tested samples is shown in FIG. 30A. There was an inverse relationship between adalimumab concentration and ATA positivity. The lower the adalimumab concentration in the patient serum, the more ATA positivity was detected. Sixty-eight percent of the serum samples were ATA positive when their adalimumab levels were below the cut point, while only 18.1% of the samples were ATA positive in patients with adalimumab levels >20 μg/mL (FIG. 30B). There was no statistically significant difference in ATA concentrations and ATA positivity between IBD, RA, and PS patient sera (FIG. 31).

TABLE 9
Patient characteristics.

| Number of patients | n = 100 | |
|---|---|---|
| Females, n (%) | 68 | (68) |
| Median age at time of sample | 50 | (22-82) |
| collection, years (range) | | |

| | | |
|---|---|---|
| Race | White | 88 |
| | Black | 9 |
| | Asian | 1 |
| | Not identified | 2 |
| Disease | CD | 49 |
| | UC | 3 |
| | RA | 33 |
| | PS | 15 |

### 4. Discussion

In the past decade, the broad use of anti-TNF-α therapy in IBD and other immune-mediated diseases has dramatically improved therapeutic outcomes (Hanauer et al., Maintenance infliximab for Crohn's disease: the ACCENT I randomised trial, Lancet. 359 1541-1549 (2002); Targan et al., A short-term study of chimeric monoclonal antibody cA2 to tumor necrosis factor alpha for Crohn's disease. Crohn's Disease cA2 Study Group, N. Engl. J. Med. 337 1029-1035 (1997); Colombel et al., Infliximab, azathioprine, or combination therapy for Crohn's disease, N. Engl. J. Med. 362 1383-1395 (2010); Present et al., Infliximab for the treatment of fistulas in patients with Crohn's disease, N. Engl. J. Med. 340 1398-1405 (1999); Rutgeerts et al., Efficacy and safety of retreatment with anti-tumor necrosis factor antibody (infliximab) to maintain remission in Crohn's disease, Gastroenterology. 117 761-769 (1999)). Nevertheless, in clinical trials for all anti-TNF-α biologics, there is a significant number of patients (>30%) who fail to respond to treatment (primary non-responders) because of different immunoinflammatory mechanisms, disease stages, pharmacokinetics, the presence of innate neutralizing anti-TNF-α antibodies and the genetic or serological background of individual patients (Hanauer et al., Maintenance infliximab for Crohn's disease: the ACCENT I randomised trial, Lancet. 359 1541-1549 (2002); Hanauer et al., Human anti-tumor necrosis factor monoclonal antibody (adalimumab) in Crohn's disease: the CLASSIC-I trial, Gastroenterology, 130 323-333 (2006); Sandborn et al., Adalimumab for maintenance treatment of Crohn's disease: results of the CLASSIC II trial, Gut. 56 1232-1239 (2007); Colombel et al., Adalimumab for maintenance of clinical response and remission in patients with Crohn's disease: the CHARM trial, Gastroenterology, 132 52-65 (2007); Schreiber et al., Maintenance therapy with certolizumab pegol for Crohn's disease, N. Engl. J. Med. 357 239-250 (2007); Wong et al., TNFalpha blockade in human diseases: mechanisms and future directions, Clin Immunol. 126 121-136 (2008)). In contrast to primary non-responders, secondary non-responders are defined by recurrence of disease activity during maintenance therapy after having achieved an initial response (Yanai et al., Assessing response and loss of response to biological therapies in IBD, Am J Gastroenterol. 106 685-698 (2011)). Many factors may contribute to the loss of response to anti-TNF-α biologics, including drug bioavailability, pharmacokinetics, immunogenicity or other factors that increase drug clearance (Maser et al., Association of trough serum infliximab to clinical outcome after scheduled maintenance treatment for Crohn's disease, Clin Gastroenterol Hepatol. 4 1248-1254 (2006); Bendtzen et al., Individualized monitoring of drug bioavailability and immunogenicity in rheumatoid arthritis patients treated with the tumor necrosis factor alpha inhibitor infliximab, Arthritis Rheum. 54 3782-3789 (2006)). Immunogenicity is associated with the generation of anti-drug antibodies which may impact both pharmacokinetic and pharmacodynamic activities. Even fully humanized monoclonal antibodies such as adalimumab may elicit the development of anti-drug antibodies (Wolbink et al., Dealing with immunogenicity of biologicals: assessment and clinical relevance, Curr Opin Rheumatol, 21 211-215 (2009); West et al., Immunogenicity negatively influences the outcome of adalimumab treatment in Crohn's disease, Aliment Pharmacol Ther. 28 1122-1126 (2008)). Anti-drug antibody formation negatively affects drug efficacy by increasing the clearance of the drug and/or neutralizing its activity, thereby reducing the amount of active drug in circulation (Baert et al., Influence of immunogenicity on the long-term efficacy of infliximab in Crohn's disease, N. Engl. J Med. 348 601-608 (2003); Hanauer et al., Incidence and importance of antibody responses to infliximab after maintenance or episodic treatment in Crohn's disease, Clin Gastroenterol. Hepatol. 2 542-553 (2004); Farrell et al., Intravenous hydrocortisone premedication reduces antibodies to infliximab in Crohn's disease: a randomized controlled trial, Gastroenterology. 124 917-924 (2003); Miele et al., Human antichimeric antibody in children and young adults with inflammatory bowel disease receiving infliximab, J Pediatr Gastroenterol. Nutr. 38 502-508 (2004)). In addition, presence of anti-drug antibodies has been associated with increased risk of infusion or injection reactions, which in turn may lead to decreased serum drug levels and shorter duration of response (Baert et al., Influence of immunogenicity on the long-term efficacy of infliximab in Crohn's disease, N. Engl. J. Med. 348 601-608 (2003); Wolbink et al., Dealing with immunogenicity of biologicals: assessment and clinical relevance, Curr. Opin Rheumatol. 21 211-215 (2009); Vermeire et al., Effectiveness of concomitant immunosuppressive therapy in suppressing the formation of antibodies to infliximab in Crohn's disease, Gut. 56 1226-1231 (2007)). Favorable clinical outcome appears to be associated with a sustained presence of therapeutic drug level (Maser et al., Association of trough serum infliximab to clinical outcome after scheduled maintenance treatment for Crohn's disease, Clin Gastroenterol Hepatol. 4 1248-1254 (2006); Farrell et al., Intravenous hydrocortisone premedication reduces antibodies to infliximab in Crohn's disease: a randomized controlled trial, Gastroenterology. 124 917-924 (2003)). When anti-drug antibody against a single biological agent is induced and the patient starts to relapse, clinical response can be regained by introduction of an alternative biological agent of the same or different class (Yanai et al., Assessing response and loss of response to biological therapies in IBD, Am J Gastmenterol. 106 685-698 (2011)). However, in the absence of direct measurement of drug levels and anti-drug antibodies, the clinician would only rely on his/her own clinical judgment to assess the mechanisms of loss of response, and the decision on the choice of a second-line biological agent is purely empirical.

Several clinical studies have shown that the measurement of trough infliximab and antibodies-to-infliximab levels in serum, with commercially available tests may help the management of patients who are losing response to infliximab treatment (Baert et al., Influence of immunogenicity on the long-term efficacy of infliximab in Crohn's disease, N. Engl. J. Med. 348 601-608 (2003); Maser et al., Association of trough serum infliximab to clinical outcome after scheduled maintenance treatment for Crohn's disease, Clin Gastroenterol Hepatol. 4 1248-1254 (2006); Vermeire et al., Effectiveness of concomitant immunosuppressive therapy in suppressing the formation of antibodies to infliximab in Crohn's disease, Gut. 56 1226-1231 (2007)). Unfortunately, for patients who are losing response to adalimumab treatment, commercial assays are not yet available for the determination of adalimumab trough levels and anti-drug antibody formation. Significantly, the reported incidence of generating ATA in adalimumab-treated patients varies significantly based on different (non-commercial) assay methods (Karmiris et al., Influence of trough serum levels and immunogenicity on long-term outcome of adalimumab therapy in Crohn's disease, Gastroenterology. 137 1628-1640 (2009); Bartelds et al., Development of antidrug antibodies against adalimumab and association with disease activity and treatment failure during long-term follow-up, JAMA. 305 1460-1468 (2011); West et al., Immunogenicity negatively influences the outcome of adalimumab treatment in Crohn's disease, Aliment. Pharmacol Ther. 28 1122-1126 (2008); Radstake et al., Formation of antibodies against infliximab and adalimumab strongly correlates with functional drug levels and clinical responses in rheumatoid arthritis, Ann Rheum. Dis. 68 1739-1745 (2009)). The discrepancies observed in these reports are likely due to different assay technologies and the timing of blood drawn, as well as the clinical assessments of patient conditions. Most of the available assays for the assessment of anti-drug antibodies in serum are based on solid phase enzyme immunoassay methodology (ELISA). These assays differ significantly in clinical specificity and sensitivity with high risk of false-positives due to non-specific binding to immunoglobulin from serum substances other than the drug. Furthermore, these assays cannot detect all the different forms of antibodies nor can they measure the free anti-drug antibodies if an excess of drug is present in the serum (Baert et al., Influence of immunogenicity on the long-term efficacy of infliximab in Crohn's disease, N. Engl. J Med. 348 601-608 (2003); Wolbink et al., Dealing with immunogenicity of biologicals: assessment and clinical relevance, Curr Opin Rheumatol. 21 211-215 (2009)). In these assays, anti-drug antibodies are considered to be positive in a patient's serum sample if the antibody concentration is above the cut point value of the test in association with undetectable drug levels. The presence of anti-drug antibodies is considered to be negative if both anti-drug antibody and drug levels are below the cut point values of the test. The test results are reported as inconclusive if the drug concentration is above the cut point value while the anti-drug antibody is undetectable (Baert et al., Influence of immunogenicity on the long-term efficacy of infliximab in Crohn's disease, N. Engl. J Med. 348 601-608 (2003)). RIA has also been developed to measure serum ATA and adalimumab concentrations and this assay has shown some advantages over ELISA (Bendtzen et al., Individualized monitoring of drug bioavailability and immunogenicity in rheumatoid arthritis patients treated with the tumor necrosis factor alpha inhibitor infliximab, Arthritis Rheum. 54 3782-3789 (2006); Wolbink et al., Development of antiinfliximab antibodies and relationship to clinical response in patients with rheumatoid arthritis, Arthritis Rheum. 54 711-715 (2006); Svenson et al., Monitoring patients treated with anti-TNF-alpha biopharmaceuticals: assessing serum infliximab and anti-infliximab antibodies, Rheumatology (Oxford), 46 1828-1834 (2007)). However, the inherent RIA methodology is more cumbersome and commercially unavailable, and the use of radioactive materials is also a major drawback in many clinical labs.

Recently, we developed a liquid-phase HMSA method to quantitatively measure both infliximab drug and antibodies-to-infliximab (ATI) levels in IBD patient serum samples (Wang et al., Development and validation of a homogeneous mobility shift assay for the measurement of infliximab and antibodies-to-infliximab levels in patient serum, J Immunol Methods. 382 177-188 (2012)). This method overcomes many limitations encountered in the solid-phase ELISA and RIA methods. Validation of the ATI-HMSA and the infliximab-HMSA showed higher sensitivity and drug tolerance compared to the ELISA method. The method was based on the incubation of infliximab-treated IBD patient serum samples with either fluorescent-labeled infliximab in order to detect ATI levels, or with fluorescent-labeled TNF-α in order to detect infliximab levels. The immune complexes formed in the incubation mixture were separated from the free label by SE-HPLC and the amount of ATI or infliximab in the samples was calculated from the resolved peak areas. Due to the superiority of HMSA over ELISA, we have applied this methodology to measure adalimumab and ATA in serum samples collected from patients treated with adalimumab. Analytical validation of the ATA-HMSA and the adalimumab-HMSA showed that the assay performance was robust and not affected by potential interfering substances present in serum. The analytical sensitivity for measuring ATA with the HMSA method is higher compared with the suboptimal concentration of 200-500 ng/mL achieved by the bridging ELISA. The ATA-HMSA has a high drug level tolerance (up to 20 μg/mL) which is suitable for the measurement of ATA in serum samples from patients under the biweekly dosing regimen. Assay cut points for the adalimumab-HMSA and the ATA-HMSA were calculated by testing 100 serum samples collected from adalimumab drug-naïve healthy subjects. The calculated clinical specificity for the adalimumab-HMSA and ATA-HMSA was 97% and 99%, respectively.

The newly developed ATA-HMSA and adalimumab-HMSA were applied to measure ATA and adalimumab concentrations in serum samples from IBD, RA, and PS patients who had lost response to adalimumab treatment. The results showed that there is higher incidence of ATA positivity (44%) in these secondary non-responding patients compare to all patients treated with adalimumab (Bartelds et al., Development of antidrug antibodies against adalimumab and association with disease activity and treatment failure during long-term follow-up, JAMA. 305 1460-1468 (2011); West et al., Immunogenicity negatively influences the outcome of adalimumab treatment in Crohn's disease, Aliment. Pharmacol Ther. 28 1122-1126 (2008); Bartelds et al., Clinical response to adalimumab: relationship to anti-adalimumab antibodies and serum adalimumab concentrations in rheumatoid arthritis, Ann Rheum. Dis. 66 921-926 (2007)). Twenty-six percent of the serum samples were found to be adalimumab drug negative (below cut point), which was unusual for patients under a biweekly dosing regimen. One of the reasons to explain this finding could be ATA generation since 68% of these patients were found to be ATA positive. ATA may increase adalimumab clearance by forming immune complexes which are cleared by immunoglobulin Fcγ receptors. ATA positivity was significantly lower (18.1%) when the adalimumab concentration in the serum was above 20 μg/mL, and the loss of response to adalimumab treatment in this patient population may not be related to ATA formation. Switching to another class of medication may be beneficial in this population subgroup of patients (Afif et al., Clinical utility of measuring infliximab and human anti-chimeric antibody concentrations in patients with inflammatory bowel disease, Am. J. Gastroenterol. 105 1133-1139 (2010)). Our novel ATA-HMSA is able to detect ATA in serum samples with adalimumab drug level up to 20 μg/mL, which is a significant improvement over the bridging ELISA method. Our study also shows that there is an inverse relationship between adalimumab concentration and ATA positivity (a greater number of ATA positive samples were detected in the patient population with low adalimumab concentrations). We also compared the ATA positivity among the IBD, RA, and PS patients. There was no significant difference in ATA concentrations and ATA positivity between these patient sera.

### 5. Conclusions

In conclusion, the ATA-HMSA and the adalimumab-HMSA are novel methods for measuring ATA and adalimumab levels in patient serum samples, and overcome many limitations encountered in solid-phase ELISA and RIA methods. The ATA-HMSA can be used to monitor anti-drug antibody formation even in the presence of high serum drug levels. Incorporation of routine measurement of anti-drug antibody and drug levels in patients treated with protein therapeutics is clinically useful and may help to optimize patient management. Prospective randomized clinical studies in different chronic inflammatory diseases are being conducted.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method for determining the presence or level of an anti-TNFα drug in a sample, the method comprising:
(a) contacting a labeled TNFα with a sample having an anti-TNFα drug to form a labeled complex with the anti-TNFα drug;
(b) subjecting the labeled complex to size exclusion chromatography to separate the labeled complex from free labeled TNFα and to measure the amount of the labeled complex and the amount of the free labeled TNFα;
(c) calculating a ratio of the amount of the labeled complex to the sum of the labeled complex plus free labeled TNFα; and
(d) comparing the ratio calculated in step (c) to a standard curve of known amounts of the anti-TNFα drug, thereby determining the presence or level of the anti-TNFα drug.

2. The method of claim 1, wherein the standard curve is generated by incubating the labeled TNFα with known amounts of the anti-TNFα drug.

3. The method of claim 1, wherein the standard curve has a y-axis comprising the ratio of labeled complex to the sum of the amount of the labeled complex plus free labeled TNFα and an x-axis comprising known amounts of anti-TNFα drug.

4. The method of claim 1, wherein the sample is serum.

5. The method of claim 1, wherein the anti-TNFα drug is a member selected from the group consisting of REMICADE™ (infliximab), ENBREL™ (etanercept), HUMIRA™ (adalimumab), CIMZIA® (certolizumab pegol), and combinations thereof.

6. The method of claim 1, wherein the size exclusion chromatography is size exclusion-high performance liquid chromatography (SE-HPLC).

7. The method of claim 1, wherein the labeled TNFα is a fluorophore-labeled TNFα.

8. The method of claim 7, wherein the fluorophore is an Alexa Fluor® dye.

9. The method of claim 1, wherein the labeled complex is eluted first, followed by the free labeled TNFα.

10. The method of claim 1, wherein the sample is obtained from a subject receiving therapy with the anti-TNFα drug.

11. A method for determining the presence or level of an autoantibody to an anti-TNFα drug in a sample, the method comprising:
(a) contacting a labeled anti-TNFα drug with the sample to form a labeled complex with the autoantibody;
(b) subjecting the labeled complex to size exclusion chromatography to separate the labeled complex from free labeled anti-TNFα drug and to measure the amount of the labeled complex and the amount of the free labeled anti-TNFα drug;
(c) calculating a ratio of the amount of the labeled complex to the sum of the amount of the labeled complex plus free labeled anti-TNFα drug; and
(d) comparing the ratio calculated in step (c) to a standard curve of known amounts of the autoantibody, to thereby determine the presence or level of the autoantibody.

12. The method of claim 11, wherein the standard curve is generated by incubating the labeled anti-TNFα drug with serum positive for the autoantibody.

13. The method of claim 11, wherein the standard curve has a y-axis comprising the ratio of the amount of labeled complex to the sum of the amount of the labeled complex plus free labeled anti-TNFα drug and an x-axis comprising known amounts of the autoantibody.

14. The method of claim 11, wherein the sample is serum.

15. The method of claim 11, wherein the sample is incubated with acid prior to admixing labeled anti-TNFα drug to dissociate any unlabeled anti-TNFα drug and autoantibody complex.

16. The method of claim 11, wherein the anti-TNFα drug is a member selected from the group consisting of REMICADE™ (infliximab), ENBREL™ (etanercept), HUMIRA™ (adalimumab), CIMZIA® (certolizumab pegol), and combinations thereof.

17. The method of claim 11, wherein the autoantibody is a member selected from the group consisting of a human anti-mouse antibody (HAMA), a human anti-chimeric antibody (HACA), a human anti-humanized antibody (HAHA), and combinations thereof.

18. The method of claim 11, wherein the size exclusion chromatography is size exclusion-high performance liquid chromatography (SE-HPLC).

19. The method of claim 11, wherein the labeled anti-TNFα drug is a fluorophore-labeled anti-TNFα drug.

20. The method of claim 19, wherein the fluorophore is an Alexa Fluor® dye.

21. The method of claim 11, wherein the labeled complex is eluted first, followed by the free labeled anti-TNFα drug.

22. The method of claim 11, wherein the sample is obtained from a subject receiving therapy with the anti-TNFα drug.

23. The method of claim 11, wherein alternatively, a ratio of the free labeled anti-TNFα drug to an internal control is determined and used to extrapolate the level of the autoantibody from the standard curve.

24. A method for determining the total amount of autoantibody in a sample, the method comprising:
(a) determining the level of autoantibody by:
(i) contacting a labeled anti-TNFα drug with the sample to form a labeled complex with the autoantibody;
(ii) subjecting the labeled complex to size exclusion chromatography to separate the labeled complex from free labeled anti-TNFα drug and to measure the amount of the labeled complex and the amount of the free labeled anti-TNFα drug;
(iii) calculating a ratio of the amount of the labeled complex to the sum of the amount of the labeled complex plus free labeled anti-TNFα drug;
(iv) comparing the ratio calculated in step (c) to a standard curve of known amounts of the autoantibody, to thereby determine the presence or level of the autoantibody, bound to a labeled anti-TNFα drug; and
(b) adding the amount of autoantibody bound to unlabeled anti-TNFα drug to the level determined in step (a) to produce the total amount of autoantibody in the sample.

25. The method of claim 24, wherein the amount of autoantibody bound to unlabeled anti-TNFα drug is calculated by multiplying the level of autoantibody bound to labeled anti-TNFα drug of step (a) by the amount of unlabeled anti-TNFα drug divided by the amount of labeled anti-TNFα drug.

26. The method of claim 25, wherein the amount of unlabeled anti-TNFα drug is the weight of anti-TNFα drug determined by multiplying the concentration of anti-TNFα drug by the volume of sample.

27. The method of claim 25, wherein the amount of labeled anti-TNFα drug is the weight of labeled anti-TNFα drug determined by multiplying the volume of labeled anti-TNFα drug by the concentration of labeled anti-TNFα drug added to the sample.
